(19) United States
(12) Patent Application Publication
(10) Pub. No.: US 2023/0063416 A1
(43) Pub. Date: Mar. 2, 2023

(54) Polytherapy Modulating Cathelicidin Gene Expression Modulation for The Treatment of Alzheimer's Disease and Other Conditions

(71) Applicants: Maxwell Biosciences, Inc., Austin, TX (US); The Board of Trustees of the Leland Stanford Junior University, Stanford, CA (US)

(72) Inventors: Annelise E. Barron, Woodside, CA (US); Andrew K. Evans, La Honda, CA (US); Jennifer S. Lin, Mountain View, CA (US); Joshua McClure, Westlake Hills, TX (US); Mehrdad Shamloo, Palo Alto, CA (US)

(73) Assignees: Maxwell Biosciences, Inc., Austin, TX (US); The Board of Trustees of the Leland Stanford Junior University, Stanford, CA (US)

(21) Appl. No.: 18/045,793

(22) Filed: Oct. 11, 2022

Related U.S. Application Data
(63) Continuation of application No. 16/038,158, filed on Jul. 17, 2018, now abandoned.
(60) Provisional application No. 62/533,589, filed on Jul. 17, 2017.

(51) Int. Cl. A61K 31/192; G01N 33/68; A61P 25/28; A61K 31/593; A61K 31/12; A61K 31/20; A61K 36/889; A61K 31/07

(52) U.S. Cl. CPC A61K 31/192; G01N 33/6896; A61P 25/28; A61K 31/593; A61K 31/12; A61K 31/20; A61K 36/889; A61K 31/07; A61P 35/00

(57) ABSTRACT

A polytherapy of orally available compounds is disclosed that synergistically modulates and induces the expression of the cathelicidin gene (CAMP), which encodes the host defense peptide LL-37. By providing a number of different CAMP-inducing compounds together at the same time, stronger gene induction is achieved than with just one or two compounds, because the mechanism of induction broadens. Induction also may vary in different pas of the body depending on which compounds are used, and at what levels. We show for the first time that the polytherapy can induce cathelicidin expression in the brain, which may help to treat or prevent Alzheimer's Disease Systemic cathelicidin gene induction may help treat numerous other conditions including Type 2 Diabetes/Metabolic Syndrome, or chronic bacterial, viral, or fungal infections associated with increased cancer risk or neurodegeneration. By increasing cellular autophagy and macroautophagy and supporting mitochondrial biogenesis and homeostasis, CAMP gene upregulation may reduce the effects of cellular aging and increase longevity.

This application claims priority from U.S. Provisional Application No. 62/533,589, filed Jul. 17, 2017, having the same tide, and having the same inventors, and which is incorporated herein by reference in its entirely.

### FIELD OF THE DISCLOSURE

The present disclosure relates generally to gene expression modulation, and more particularly to polytherapy which modulates cathelicidin gene expression for the treatment of medical conditions such as Alzheimer's disease.

### BACKGROUND OF THE DISCLOSURE

### Initial Discovery of Mammalian Cathelicidins, and Human Cathelicidin LL-37.

In 1991, Agerberth et al first discovered the cathelicidin peptide and reported its antibacterial activity. That fire literature report appeared in December 1991 and described the porcine cathelicidin PR-39. [REF: Amino acid sequence of PR-39. Isolation from pig intestine of a new member of the family of proline-arginine-rich antibacterial peptides. Agerberth B, Lee J Y, Bergman T, Carlquist M, Boman H G, Mutt V, Jörnvall H. Eur J Biochem. 1991 Dec. 18; 202(3):849-54. Pubmed ID PMID: 1765598] The first studies of the mechanisms of antibacterial action of PR-39 were reported in 1993. [REF: Mechanisms of action on Escherichia coli of cecropin P1 and PR-39, two antibacterial peptides from pig intestine. Boman H G, Agerberth B, Boman A. Infect Immun. 1993 July; 61(7):2978-84. Pubmed ID PMID: 8514403]

In 1995, the first report of the human peptide analogue of PR-39 was reported by the same group. It was originally called FALL-39, but the name was changed later to LL-37. [REF: FALL-39, a putative human peptide antibiotic, is cysteine-free and expressed in bone marrow and testis. Agerberth B, Gunne H, Odeberg J, Kogner P, Boman H G, Gudmundsson G H. Proc Natl Acad Sci USA. 1995 Jan. 3; 92(1):195-9. Pubmed PMID: 7529412] Also in 1995, the rabbit cathelicidin protein was reported by Larrick et al., and was shown to bind to and detoxify bacterial lipopolysaccharide (LPS), which is a dangerous proinflammatory molecule in infections. [REF: Human CAP18: a novel antimicrobial lipopolysaccharide-binding protein. Larrick J W, Hirata M, Balint R F, Lee J, Zhong J, Wright S C. Infect Immun. 1995 April; 63(4):1291-7. Pubmed ID PMID 7890387].

The human cathelicidin gene was initially called FALL39 by is Swedish discoverer, and later renamed CAMP. Human cathelicidin protein initially was shown to be expressed and processed to its final, active peptide form, LL-37, in human granulocytes (such as neutrophils), which are mature white blood cells that contain, in their cytoplasm, granules of antipathogenic peptides, such as LL-37 and defensins. [REF: The human gee FALL9 and processing of the cathelin precursor to the antibacterial peptide LL-37 in granulocytes. Gudmundsson G H, Agerberth B, Odeberg J, Bergman T, Olsson B, Salcedo R. Eur J Biochem. 1996 Jun. 1; 238(2):325-32. Pubmed ID PMID: 8681941]

It is to be noted that granulocytes are sometimes also called Polymorphonuclear Leukocytes (variously abbreviated PMN, PML, or PMNL), and include many immune cell types in the human system, including for example neutrophils, macrophages, B cells, and T cells. [REF: The human antimicrobial and chemotactic peptides LL-37 and alpha-defensins are expressed by specific lymphocyte and monocyte populations. Agerberth B, Charo J, Werr J, Olsson B, Idali F, Lindbom L, Kiessling R, Jörnvall H, Wigzell H, Gudmundsson G H. Blood 2000 Nov. 1; 6(9):3486-93. Pubmed ID PMID: 11049988] The effects of LL-37 on a number of different human cell types, including human peripheral blood mononuclear cells, CD14+ monocytes, myeloid and plasmocytoid dendritic cells and T- and B-lymphocytes, was studied by flow cytometry and DNA microarrays, and was shown to be both significant and complex, with all cell types responding to treatment with LL-37 peptide. [REF: Mol Biosyst. 2009 May; 5(5):483-96. Systems biology evaluation of immune responses induced by human host defence peptide LL-37 in mononuclear cells. Mookherjee N I, Hamill P, Gardy J, Blimkie D, Falsafi R, Chikatamarla A, Arenillas D J, Doria S, Kollmann T R, Hancock R E. Pubmed PMID 19381363] In other words, white blood cells make and release LL-37 as an antimicrobial peptide. Once released, however, the LL-37 peptide is also immunomodulatory of white blood cell phenotype.

### The Human LL-37 Gene CAMP and LL-37 Mechanism of Antibacterial Action.

In 1997 it was reported that the expression of the human gene encoding LL-37 is induced in keratinocytes during inflammatory disorders, highlighting the complexity of LL-37's in vivo immunomodulatory functions. [REF: The expression of the gene coding for the antibacterial peptide LL-37 is induced in human keratinocytes during inflammatory disorders. Frohm M, Agerberth B, Ahangari G, Stáhle-Bäckdahl M, Lidén S, Wigzell H, Gudmundsson G H. J Biol Chem. 1997 Jun. 13; 272(24):15258-63. Pubmed PMID: 9182550] In 1998, further studies of the antibacterial activity and mechanism of LL-37 were published, as interest in this peptide as a broad-spectrum endogenous antibiotic peptide grew. [REF: Conformation-dependent antibacterial activity of the naturally occurring human peptide LL-37 Johansson J, Gudmundsson G H, Rottenberg M E, Berndt K D, Agerberth B. J Biol Chem. 1998 Feb. 6; 273(6):3718-24. Pubmed ID PMID: 9452503] and [REF: Structure and activity of cathelicidin antibacterial proteins. Wang Y, Agerberth B, Johansson J. J Protean Chem. 1998 August; 17(6):522-3. Pubmed ID PMID: 9723727] Initial reports focused on bacterial membrane binding and disruption as the predominant mechanism of bacterial killing, but a 2017 report showed that LL-37 peptides rapidly cross bacterial cell membranes and have profound intracellular effects, aggregating cytosolic bacterial biomolecules irreversibly. [REF: Intracellular biomass flocculation as a key mechanism of rapid bacterial killing by cationic, amphipathic antimicrobial peptides and peptoids. Chongsíríwatana N P, Lin J S, Kapoor R, Wetzler M, Rea J A C, Didwania M K, Contag C H, Barron A E. Sci Rep 2017 Dec. 1; 7(1):16718. doi: 10.1038/a41598-017-16180-0. Pubmed ID PMID: 29196622] Injury to the skin has been shown to cause the expression and release of cathelicidin, which then can prevent infection by group A Streptococcus, a common skin pathogen. [REF: Cutaneous injury induces the release of cathelicidin anti-microbial peptides active against group A Streptococcus. Dorschner R A, Pestonjamasp V K, Tamakuwala S, Ohtake T, Rudisill J, Nizet V, Agerberth B, Gudmundsson G H, Gallo R L. J Invest Dermatol. 2001 July; 117(1).91-7. Pubmed ID PMID: 11442754]

### LL-37 is a Critical Element of Both the Formation and Clearance of Extracellular Traps

Another important aspect of the human body's response to invasion by pathogens is the formation of Neutrophil Extracellular Traps (NETs). [REF: Beneficial suicide: why neutrophils die to make NETs. Brinkmann V, Zychlinsky A. Nat Rev Microbiol. 2007 August; 5(8).577-82. Pubmed ID PMID 17632569] Neutrophils, which are the most abundant class of white blood cells, upon stimulation (e.g., by the presence of bacteria) not only engulf pathogens, but can also produce extracellular structures called NETs. These structures, which are mostly DNA-based, directly capture and kill microorganisms, functioning like antibiotic spider webs. Other types of white blood cells with more specialized functions, such as eosinophils, are also able to make extracellular traps, which immobilize and subsequently kill pathogens. [REF: Catapult-like release of mitochondrial DNA by eosinophils contributes to antibacterial defense. Yousefi S, Gold J A, Andina N, Lee J J, Kelly A M, Kozlowski E, Schmid I, Straumann A, Reichenbach J, Gleich G J, Simon H U. Nat Med. 2008 September; 14(9).949-53. Pubmed ID PMID 18690244]

Mast cells we also able to make extracellular traps, and presumably other cell types as well. [REF: Phagocytosis-independent antimicrobial activity of mast cells by means of extracellular trap formation. von Köckritz-Blickwede M, Goldmann O, Thulin P, Heinemann K, Norrby-Teglund A, Rohde M, Medina E. Blood. 2008 Mar. 15; 111(6):3070-80. Pubmed ID PMID 18182576] NETs have been shown to be formed primarily from human DNA molecules, which are physically crosslinked by two classes of cationic proteins: histones (which function to spool DNA within the nucleus) and the cathelicidin LL-37. Thus, LL-37 plays an important role in stabilizing extracellular traps, which are an essential pot of the human innate immune response to infection. [REF: The antimicrobial peptide LL-37 facilitates the formation of neutrophil extracellular traps. Neumann A, Berends E T, Nerlich A, Molhoek E M, Gallo R L, Meerloo T, Nizet V, Naim H Y, von Köckritz-Blickwede M. Biochem J. 2014 Nov. 15:464(1):3-11. Pubmed PMID: 25181554] and [REF: Novel role of the antimicrobial peptide LL-37 in the protection of neutrophil extracellular traps against degradation by bacterial nucleases. Neumann A, Völlger L, Berends F T, Molhoek E M, Stapels D A, Midon M, Friàes A, Pingoud A, Rooijakkers S H, Gallo R L, Mörgelin M, Nizet V, Naim H Y, von Köckritz-Blickwede M. J Innate Immun. 2014; 6(6):860-8. Pubmed PMID: 25012862]

Some pathogens that escape killing by NETs, do so by releasing M1 peptides (known as virulence factors) that specifically bind to and inactivate LL-37. [REF: M1 protein allows Group A streptococcal survival in phagocyte extracellular traps through cathelicidin inhibition. Lauth X I, von Köckritz-Blickwede M, McNamara C W, Myskowski S, Zinkernagel A S, Beall B, Ghosh P, Gallo R L, Nizet V. J Innate Immun. 2009; 1(3):202-14. Pubmed ID PMID 20375578] Impaired clearance and degradation of extracellular traps, which reflects a dysfunction of phagocytosis and macroautophagy, occurs in several types of chronic autoimmune diseases, including dermatomyositis and polymyositis. [REF: Enhanced formation and impaired degradation of neutrophil extracellular traps in dermatomyositis and polymyositis: a potential contributor to interstitial lung disease complications. Zhang S, Shu X, Tian X, Chen F, Lu X, Wang G. Clin Exp Immunol. 2014 July; 177(1):134-41. Pubmed PMID: 24611519] and [REF: Neutrophil extracellular traps and their role in the development of chronic inflammation and autoimmunity. Pinegin B, Vorobjeva N, Pinegin V. Autoimmun Rev. 2015 July; 14(7):633-40. Review. Pubmed PMID: 25797532]

In Systemic Lupus Erythematosus, uncleared NETs appear to be a critical autoimmune irritant, which are involved in the chronic disease inflammatory process. [REF: Neutrophils activate plasmacytoid dendritic cells by releasing self-DNA-peptide complexes in systemic lupus erythematosus. Lande R, Ganguly D, Facchinetti V, Frasca L, Conrad C, Gregorio J, Meller S, Chamilos G, Sebasigari R, Riccieri V, Bassett R, Amuro H, Fukuhara S, Ito T, Liu Y J, Gilliet M. Sci Transl Med. 2011 Mar. 9; 3(73):73ra19. Pubmed ID PMID 21389263] and [REF: Netting neutrophils ae major inducers of type I IFN production in pediatric systemic lupus erythematosus. Garcia-Ramo G S I, Caielli S, Vega B, Connolly J, Allantaz F, Xu Z, Punaro M, Baisch J, Guiducci C, Coffman R L, Barrat F J, Banchereau J, Pascual V. Sci Transl Med. 2011 Mar. 9; 3(73):73ra20. Pubmed ID PMID 21389264] This also appears to be the case in the autoimmune skin condition psoriasis, for which it was found that complexes between self-DNA (from the person's own damaged skin cells) and LL-37 induce autoimmune inflammation in the skin. [REF: Plasmacytoid dendritic cells sense self-DNA coupled with antimicrobial peptide. Lande R, Gregorio J, Facchinetti V, Chatterjee B, Wang Y H, Homey B, Cao W, Wang Y H, Su B, Nestle F O, Zal T, Mellman I, Schroder J M, Liu Y J, Gilliet M. Nature 2007 Oct. 4; 444(7162):564-9. Pubmed ID PMID 17873860]

As will be discussed in more detail below, LL-37 is also a critical player in the process of macroautophagy, i.e., the clearance of extracellular debris and inactivated or killed pathogens by macrophages, which is the body's way of clearing away extracellular traps, once their purpose has been served. [REF: Vitamin D3 induces autophagy in human monocytes/macrophages via cathelicidin. Yuk J M, Shin D M, Lee H M, Yang C S, Jin H S, Kim K K, Lee Z W, Lee S H, Kim J M, Jo E K. Cell Host Microbe. 2009 Sep. 17; 6(3):231-43. Pubmed ID PMID: 19748465] and [REF: Antimicrobial peptide LL-37 promotes bacterial phagocytosis by human macrophages. Wan M, van der Does A M, Tang X, Lindbom L, Agerberth B, Haeggström J Z. J Leukoc Biol. 2014 June; 95(6):971-81. Pubmed ID PMID: 24550523] Thus, as discussed above, LL-37 plays at least three distinct roles in the control of pathogens by extracellular traps: (1) LL-37 directly crosslinks and stabilizes NETs; (2) LL-37 directly kills pathogens trapped in NETs; and (3) LL-37 stimulates autophagic clearance of NETs by macrophages. Thus, the expression of LL-37 is important for many aspects of human innate immune response to pathogens and to clearance of damaged host cell debris and NETs as well.

### LL-37 is Antifungal.

In addition to being antibacterial, the LL-37 peptide is antifungal. A fungal species that is often responsible for acute or chronic infections in human patients, particularly in the human oral cavity and urogenital tract, is Candida albicans. For instance, patients who have been treated with radiation or chemotherapy for cancer, or AIDs patients who are immunosuppressed, often suffer from painful fungal infections of the mouth. The LL-37 peptide has been shown to be antifungal, by preventing Candida albicans' adhesion to tissue and cell surfaces, via binding to the fungal carbohydrate mannan. [REF: Human antimicrobial peptide LL-37 inhibits adhesion of Candida albicans by interacting with yeast cell-wall carbohydrates. Tsai P W I, Yang C Y, Chang H T, Lan C Y. PLoS One 2011 Mar. 14; 6(3):e17755. Pubmed ID PMID 21448240] Moreover, the cathelicidin peptide LL-37 also directly inhibits fungal growth and kills Candida albicans cells rapidly by inducing cell membrane permeabilization, causing vacuolar expansion. [REF: Fungicidal mechanisms of cathelicidins LL-37 and CATH-2 revealed by live-cell imaging. Ordonez S R, Amarullah I H, Wubbolts R W, Veldhuizen H J, Haagsman H P Antimicrob Agents Chemother. 2014; 58(4):2240-8. Pubmed ID PMID 24492359] Mechanistic studies reveal that LL-37 is most effective at killing cutaneous, rather than subcutaneous fungal pathogens, emphasizing LL-37's essential barrier function, and the importance for human immunity to maintain a steady, normal concentration of LL-37 in the skin, in order to prevent infections. [REF: Anti-fungal activity of cathelicidins and their potential role in Candida albicans skin infection. López-Garcia B I, Lee P H, Yamasaki K, Gallo R L. J Invest Dermatol. 2005 July; 125(1):108-15. Pubmed ID PMID 15982310]

### LL-37 is Antiviral.

The human cathelicidin peptide LL-37 is also a broad-spectrum antiviral host defense peptide. For instance, LL-37 has strong activity against the Herpesvirus HSV-1 and Adenovirus Ad19; this was demonstrated in the context of the epithelium of the eye, or ocular surface [REF: Human cathelicidin (LL-37), a multifunctional peptide, is expressed by ocular surface epithelia and has potent antibacterial and antiviral activity. Gordon Y J I, Huang L C, Romanowski E G, Yates K A, Proske R J, McDermott A M. Curr Eye Res. 2005 May; 30(5):385-94. Pubmed ID PMID 16020269]

A painful skin condition known as atopic dermatitis eczema herpeticum (ADEH) can result from chronic infection and dissemination of the Herpesvirus HSV-2 in damaged skin of patients suffering from atopic dermatitis. It was shown that lower-than-normal endogenous levels of cathelicidin LL-37 predispose these patients to ADEH; and further, that LL-37 is able to inactivate the Herpesvirus HSV-2 directly. [REF: Cathelicidin deficiency predisposes to eczema herpeticum. Howell M D, Wollenberg A, Gallo R L, Flaig M, Streib J E, Wong C, Pavicie T, Boguniewicz M, Leung D Y. J Allergy Clin Immunol 2006 April; 117(4):836-41. Pubmed ID PMID 16630442]

The essential role of antimicrobial peptides, including cathelicidin, as defense against skin infections by Vaccinia virus won also demonstrated in murine studies using transgenic mice deficient in cathelicidin peptide. [REF: Skin mast cells protect mice against vaccinia virus by triggering mast cell receptor S1PR2 and releasing antimicrobial peptides. Wang Z I, Lai Y, Bernard J J, Macleod D T, Cogen A L, Moss B, Di Nardo A. J Immunol. 2012 Jan. 1; 188(1):345-37. Pubmed ID PMID 22140255] A virus that can cause severe respiratory infections, especially in infants and children but also in adults, is the Respiratory Syncytial Virus (RSV). It has been shown in both mice and humans that cathelicidins have important, direct anti-RSV protective functions in vivo. [REF: Cathelicidins Have Direct Antiviral Activity against Respiratory Syncytial Virus In Vitro and Protective Function In Vivo in Mice and Humans. Currie S M, Gwyer Findlay E, McFarlane A, Fitch P M, Böttcher B, Colegrave N, Paras A, Jozwik A, Chiu C, Schwarze J, Davidson D J. J Immunol. 2016 Mar. 15; 196(6):2699-710. Pubmed ID PMID 26873992]

Another widespread viral infection in the world's population, especially in Asia, is the Hepatitis C Virus (HCV), which affects liver tissue and can predispose those who are infected to liver cancer. The human cathelicidin LL-37 is active against HCV, and attenuates HCV infection. It has been shown that Vitamin D3 supplementation can improve the efficacy of Interferon Gamma therapy, a long-term (1-year) treatment for HCV infection of the liver, this was stated to be the result of better LL-37 expression induced by Vitamin D3 [REF: Antimicrobial peptide LL-37 attenuates infection of hepatitis C virus. Matsumura T, Sugiyama N, Murayama A, Yamada N, Shiina M, Asabe S, Wakita T, Imawari M 5, Kato T, Hepatol Res. 2016 August; 46(9):924-32. Pubmed ID PMID 26606891].

Additionally, the human cathelicidin peptide LL-37 has been demonstrated to play an important role in host defense against influenza A viruses (IAV), exerting both direct antiviral effects, by disrupting IAV viral membranes, and also modulating host inflammatory responses to infection. [REF: The human cathelicidin LL-37 inhibits influenza A viruses through a mechanism distinct from that of surfactant protein D or defensins. Tripathi S I, Tecle T, Verma A, Crouch E, White M, Hartshorn K L. J Gen Virol 2013 January; 94(Pt 1):40-9. Pubmed PMID 230S2388] and [REF: LL-37 modulates human neutrophil responses to influenza A virus. Tripathi S, Verma A, Kim E J, White M R, Hartshorn K L, J Leukoc Biol. 2014 November; 96(5):931-8. Pubmed PMID: 25082153]. LL-37 is able to directly inhibit the replication of the HIV-1 virus, and thus is important in preventing the progression of HIV infection to AIDS. [REF: The antimicrobial peptide LL-37 inhibits HIV-1 replication. Bergman P, Walter-Jallow L, Broliden K, Agerberth B, Söderlund J. Curr HIV Res. 2007 July; 5(4):410-S. PMID: 1762754].

In examining the progress of latent HIV-1 infection and looking at CD4 levels in T cells (essential for health maintenance of HIV-infected individuals), and looking at a group that had been treated with antiretroviral drugs (ARTs) vs. individuals who had not, researchers found that LL-37 levels were positively correlated with serum Vitamin D3 levels in controls, but not in HIV-infected groups. LL-37 levels were also positively correlated with current CD4 and DCD4 (current-nadir) in a previously ART-treated group. After adjusting for age, race, sex, and HIV infection duration, the association between LL-37 and CD4 levels remained significant. Thus, LL-37 levels were found to be significantly correlated with immune system strength in HIV-infected persons. [REF: LL-37 concentrations and the relationship to vitamin D, immune status, and inflammation in HIV-infected children and young adults. Tangpricha V, Judd S E, Ziegler T R, Hao L, Alvarez J A, Fitzpatrick A M, McComsey G A, Eckard A R. AIDS Res Hum Retroviruses. 2014 July; 30(7):670-6. PMID: 24799231]

Remarkably, the known natural resistance of certain individuals to HIV infection appears to be related to their higher-than-usual endogenous, natural levels of expression of LL-37. [REF: Antiviral molecules correlate with vitamin D pathway genes and are associated with natural resistance to HIV-1 infection. Aguilar-Jimenez W, Zapata W, Rugeles M T. Microbes Infect. 2016 July-August; 18(7-8):510-6. Pubmed ID PMID 27083474] Thus, HIV-infected individuals would likely benefit from maintaining the highest possible levels of the cathelicidin LL-37 in their bloodstream.

Many review articles have been written about the family of cathelicidin proteins, which discuss and consider their many different functions and physiological effects. [REF: Cathelicidins—a family of multifunctional antimicrobial peptides. Bals R, Wilson J M. Cell Mol Life Sci. 2003 April; 60(4):711-20. Pubmed ID PMID 12795718]. Signaling its high significance to human health, a number of reviews have focused solely on the human cathelicidin protein, LL-37, in particular. [REF: Pulmonary defense and the human cathelicidin hCAP-18/LL-37. Fahy R J I, Wewers M D. Immunol Res. 2005; 31(2):75-89. Pubmed ID PMID 15778507] and [REF: LL-37, the only human member of the cathelicidin family of antimicrobial peptides. Dürr U H I, Sudheendra U S, Ramamoorthy A. Biochim Biophys Acta. 2006 September; 175(9):1408-25. Pubmed ID PMID 16716248] and [REF: The chemistry and biology of LL-37. Burton M F, Steel P G. Nat Prod Rep 2009 December; 26(12):1572-44. Review. Pubmed ID PMID: 19936387] and [REF: Cathelicidin LL-37: a multitask antimicrobial peptide. Bucki R, Leszezyńska K, Namiot A, Sokolowski W. Arch Immunol Ther Exp (Warsz). 2010 February; 58(1):15-25. Pubmed ID PMID 20049649] and [REF: A comprehensive summary of LL-37, the factotum human cathelicidin peptide. Vandamme D I, Landuyt B, Luyen W, Schoofs L. Cell Immunol. 2012 November; 280(1):22-35. Pubmed ID PMID 23246832] and [REF: Unique features of human cathelicidin LL-37. Bandurska K, Berdowska A, Barczynska-Felusiak R, Krupa P. Biofactors. 2015 September-October; 41(5):289-300. Pubmed ID PMID] These numerous review articles have been written and published because the LL-37 peptide is both important and unique in the human proteome and immune system. At this time, LL-37 is continually being discovered to play additional, complex roles in human health and disease. This makes the proper expression and regulation of the human gene that encodes LL-37, CAMP, essential to human health.

### LL-37 is an Essential Element of Cellular Autophagy, an Important Physiological Process

Besides being directly antibacterial, the peptide LL-37 is an essential participant in innate immune process known as autophagy, which includes the engulfment of pathogens such as bacteria or viruses by white blood cells such as macrophages, neutrophils, and natural killer (NK) cells. It was shown in 2009 that autophagy in human monocytes and macrophages is enhanced by the presence of Vitamin D3, specifically because Vitamin D3 enhances the expression of the cathelicidin LL-37, which was identified as being directly involved in autophagy [REF: Vitamin D3 induces autophagy in human monocytes/macrophages via cathelicidin. Yuk J M, Shin D M, Lee H M, Yang C S, Jin H S, Kim K K, Lee Z W, Lee S H, Kim J M, Jo E K. Cell Host Microbe. 2009 Sep. 17; 6(3):231-43. Pubmed ID PMID: 19748465] and [REF: Antimicrobial peptide LL-37 promotes bacterial phagocytosis by human macrophages. Wan M, van der Does A M, Tang X, Lindbom L, Agerberth B, Haeggström J Z. J Leukoc Biol. 2014 June; 95(6):971-81. Pubmed ID PMID 24550523].

This stimulation of autophagy was shown specifically in the case of mycobacteria, which cause tuberculosis. [REF: Phenylbutyrate induces LL-37-dependent autophagy and intracellular killing of Mycobacterium tuberculosis in human macrophage: Rekha R S, Rao Muvva S S, Wan M, Raqib R, Berman P, Brighenti S, Gudmundsson G H, Agerberth B. Autophagy. 2015; 11(9):1688-99. Pubmed ID PMID 26218841] Thus, the clearance of infectious pathogens by white blood cells is enhanced by the upregulation of the cathelicidin LL-37. Additionally, other diseases that would benefit from enhancement of autophagic processes might be treated by upregulation of cathelicidin.

As an example, the natural process of autophagy appears to be dysfunctional in neurodegenerative diseases. [REF: Autophagy gone awry in neurodegenerative diseases. Wong E, Cuervo A M. Nat Neurosci. 2010 July; 13(7):S05-11. Pubmed ID PMID 20581817] Examination of brain tissue from patients who died from Alzheimer's Disease showed the extensive involvement of disorders of autophagy in this chronic neurodegenerative condition. [REF: Extensive involvement of autophagy in Alzheimer disease: an immuno-electron microscopy study. Nixon R A, Wegiel J, Kumar A, Yu W H, Peterhoff C, Cataldo A, Cuervo A M. J Neuropathol Exp Neurol. 2005 February; 64(2):113-22.] Another example is in Chronic Granulomatous Disease (CGD); a deficit in autophagy appears to play a key role in the pathogenesis of granulomatous colitis in CGD [REF: Deficient autophagy unravels the ROS paradox in chronic granulomatous disease. van de Veerdonk F, Dinarello C A. Autophagy. 2014 June; 10(6):1141-2. Pubmed ID PMID 24879159] As another example, the clearance of chronic Herpesvirus infections is dependent upon macro-autophagic clearance processes and there is evidence that Herpesviruses often may evade clearance by modulating autophagy, as a way to escape elimination by innate and adaptive immune responses. [REF: Autophagy in herpesvirus immune control and immune escape. Taylor G S, Mautner J, Münz C. Herpesviridae. 2011 Jan. 5; 2(1):2. Pubmed ID PMID 21429245].

Specifically in the case of viral infections of neuronal tissue, autophagy is a critical mechanism of neuronal self-defense against viruses, since neuronal cells are long-lived in the body, according to their function in memory for instance, and cannot undergo apoptosis without harming neural functions. [REF: Autophagy and viral neuroviulence. Orvedahl A, Levine B. Cell Microbiol. 2008 September; 10(9):1747-56. Pubmed ID PMID 18503639] Thus, the treatment of chronic viral infections by pathogens such as Herpesvirus should be enhanced by LL-37 upregulation.

Remarkably, the process of autophagy has been shown to be essential for lifespan extension in model studies in animals, where various compounds were studied for that purpose. [REF: Autophagy mediates pharmacological lifespan extension by spermidine and resveratrol. Morselli E I, Galluzzi L, Kepp O, Criollo A, Maiuri M C, Tavernarakis N, Madeo F, Kroemer G. Aging (Albany N.Y.). 2009 Dec. 23; 1(12):961-70. Pubmed CD PMID 20157579]. In general, the process of autophagy is critical for healthy tissue homeostasis, especially in the case of maintaining neuronal tissue health, which can include both brain tissue and the peripheral neurons important for the senses of sight, smell, taste, and touch. [REF: Autophagy for tissue homeostasis and neuroprotection. Mariño G, Madeo F, Kroemer G. Curr Opin Cell Biol. 2011 April; 23(2):198-206. Pubmed ID PMID 21030235]. This presents the possibility that enhancement of autophagic processes, by increasing LL-37 expression above a baseline or uninduced level, would have the potential of enhancing a person's longevity and increasing the ability of peripheral neurons to provide a keen sense of sight, smell, taste, and touch into old age.

### LL-37 Increases Mitochondrial Biogenesis and Maintains Mitochondrial Homeostasis.

Relevant to the topic of longevity, LL-37 expression has been shown to protect the health of mitochondria, the energy production organelles of cells. In a HaCat cell culture system, the presence of LL-37 was demonstrated to dramatically reduce the mRNA levels and protein secretion of inflammatory cytokines including IL-6, IL-8, IL-1α and tumor necrosis factor-α (TNF-α) which are induced by lipopolysaccharides (LPS). The anti-inflammatory effects of LL-37 resulted from its demonstrated ability to increase mitochondrial biogenesis and to maintain mitochondrial homeostasis. [REF: LL-37 attenuates inflammatory impairment via mTOR signaling-dependent mitochondrial protection Sun W, Zheng Y, Lu Z, Wang H, Feng Z, Wang J, Xiao S, Liu F, Liu J. Int J Biochem Cell Biol. 2014 September; 54:26-35. Pubmed ID PMID: 24984264]

### LL-7 Affects Stem Cells and Stimulates Wound Healing.

LL-37 is a pleiotropic peptide, i.e., it has a wide range of different biological functions and physiological effects (“pleiotropy” occurs when one gene influences two or more seemingly unrelated phenotypic traits). As early as 2004, it was understood that within the extracellular matrix (i.e., the intracellular space of human tissue), LL-37 is chemotactic (i.e., that LL-37 can induce cell migration) for neutrophils, monocytes, mast cells, and T cells. Additionally, LL-37 has been shown to induce the degranulation of mast cells; to alter transcriptional responses in macrophages and finally, to stimulate wound vascularization and the re-epithelialization of healing skin [REF: Cathelicidins, multifunctional peptides of the innate immunity. Zanetti M. J Leukoc Biol. 2004 January; 75(1):39-48. Pubmed ID PMID 12960280]

In an angiogenesis model, experiments showed that LL-37 can sensitize stem cells to become migratory at lower chemokine concentrations, enhancing neovascularization by embryonic stem cells. [REF: NF kappaB activation in embryonic endothelial progenitor cells enhances neovascularization via PSGL-1 mediated recruitment: novel role for LL-37. Pfosser A, El-Aouni C, Pfisterer I, Dietz M, Globish F, Stachel G, Trenkwalder T, Pinkenburg O, Horstkotte J, Hinkel R, Sperandio M, Hatzopoulos A K, Bockstegers P, Bals R, Kupatt C. Stem Cells. 2010 February; 28(2):376-85. Pubmed ID PMID: 20014279] In a chronic ischemic hind-limb animal model, delivery of the CAMP gene encoding LL-37 using an adenovirus vector was shown to result in enhanced wound neovascularization, when LL-37 was expressed. [REF: Recombinant adeno-associated virus-based gene transfer of cathelicidin induces therapeutic neovascularization preferentially via potent collateral growth. Pinkenburg O, Pfosser A, Hinkel R, Böttcher M, Dinges C, Lebberz C, Sultana S, Enssle J, El-Aouni C, Buning H, Boekstegers P, Bals R, Kupatt C. Hunt Gene Ther. 2009 February; 20(2):159-67. Pubmed ID PMID: 20377367].

Another study showed that LL-37 enhances angiogenesis by stimulating PGE2-EP3 signaling in endothelial cells. [Cathelicidin LL-37 induces angiogenesis via PGE2-EP3 signaling in endothelial cells, in vivo inhibition by aspirin. Salvado M D, Di Gennaro A, Lindbom L, Agerberth B, Haeggström J Z. Arterioscler Thromb Vasc Biol. 2013 August; 33(8):1965-72. Pubmed ID PMID: 23766264] Additionally, it was shown that CAMP gene expression to produce the LL-37 peptide is essential in cellular differentiation of cord blood-derived CD34+ cells into early endothelial progenitor cells, which in turn are essential for the healing and regeneration of blood vessels. [REF: Successful in vitro expansion and differentiation of cord blood derived CD34+ cells into early endothelial progenitor cells reveals highly differential gene expression. Ahrens I I, Domeij H, Topcic D, Haviv I, Merivirta R M, Agrotis A, Leitner E, Jowett J B, Bode C, Lappas M, Peter K. PLoS One. 2011; 6(8):e23210. Pubmed ID PMID 21858032] Thus, enhanced expression of LL-37, up to appropriate and normal levels, in a person suffering from wounds or who has experienced physical trauma to their tissues, may enhance the rate and improve the quality of wound healing and neovascularization.

### LL-37 is Important in Lung Health.

LL-37 is produced not only by white blood cells and granulocytes, but also by the skin and all epithelial cells types, for instance, epithelial cells that line the internal surfaces of the lungs. LL-37 is detected in bronchoalveolar lavage fluid. In epithelial cells, LL-37 is produced in response to inflammation. In the lung, the antibacterial peptide LL-37 is produced by alveolar macrophages, bronchial epithelial cells, and bronchial glands, suggesting that it has key defensive roles in airway mucosa. [REF: Antibacterial components in bronchoalveolar lavage fluid from healthy individuals and sarcoidosis patients Agerberth B, Grunewald J, Castaños-Velez E, Olason B, Jörnvall H, Wigzell H, Eklund A, Gudmundsson G H. Am J Respir Crit Care Med. 1999 July; 160(1):283-90. Pubmed ID PMID 10390413] Thus, the enhanced expression of LL-37 in patients who suffer from lung injury, insults to lung tissue including aspiration or inhalation of toxins, or lung infections involving inhalation of pathogens, could improve health outcomes for these patients.

Another dangerous bacterial lung pathogen is Pseudomonas aeruginosa. LL-37 has been shown to play important roles in lung defense against this pathogen, by increasing lung epithelial cell stiffness so that bacteria cannot cross through lung tissue. [REF: Cathelicidin LL-37 increases lung epithelial cell stiffness, decreases transepithelial permeability, and prevents epithelial invasion by Pseudomonas aeruginosa. Byfield F J, Kowalski M, Cruz K, Leszezyńska K, Namiot A, Savage P B, Bucki R, Janmey P A. J Immunol. 2011 Dec. 15; 187(12):642-9. Pubmed ID PMID 22095714] Here, it should be noted that enhancing tissue stiffness is another important aspect of LL-37's host defense. Similar protective effects are seen in blood vessel endothelial cells, so that LL-37 levels directly affect the permeability of endothelial cell barriers to pathogen invasion. [REF: Cathelicidin LL-37 peptide regulates endothelial cell stiffness and endothelial barrier permeability. Byfield F J, Wen Q, Leszezynska K, Kulakowska A, Namiot Z, Janmey P A, Bucki R. Am J Physiol Cell Physiol 2011 January; 300(1):C105-12. Pubmed ID PMID: 20943960.

LL-37 Works with Vitamin D3 to Form and Heal Bone.

LL-37 is also important in the nucleation of new bone, and may be important for the regeneration of bone tissue, as important for the prevention of osteoporosis. Bone cell types include osteoblasts, osteoclasts, and osteocytes, each of which originate from different precursor cells, and are stimulated by various growth factors to differentiate into new bone. The formation of new bone tissue is necessary, for instance, in the healing of bone fractures. Sites of bone injury will naturally recruit a white blood cell type called a monocyte. It was shown that LL-37 (which is produced from its precursor protein, hCAP-18) can cause monocytes to differentiate into large, adherent cells, which form nascent bone-like structures both in vitro and in vivo. [REF: Generation of novel bone forming cells (monoosteophils) from the cathelicidin-derived peptide LL-37 treated monocytes. Zhang Z I, Shively J E. PLoS One. 2010 Nov. 15; 5(11):e13985. Pubmed ID PMID 21085494] Thus, patients who have bone fractures that need to heal, or patients who suffer from osteoporosis, would benefit from enhanced expression of the CAMP gene that encodes LL-37.

### LL-37 can Protect Against Sexually Transmitted Disease.

The precursor protein to LL-37, hCAP-8, has been identified as being present in human seminal fluid, and appears to play an important role in natural host defense during sexual activity. [REF: Isolation of human cationic antimicrobial protein-18 from seminal plasma and its associating with prostasomes. Andersson E I, Sørensen O E, Frohm B, Borregaard N, Egesten A, Malm J. Hum Reprod. 2002 October; 17(10):2529-34. Pubmed ID PMID 12351523]. People with higher endogenous levels of LL-37 might be less likely to become infected by sexually transmitted diseases. HIV has already been discussed above, but another sexually transmitted pathogen is Chlamydia trachomatis, an obligate intracellular bacterial pathogen. The human cathelicidin LL-37 has demonstrated activity against Chlamydia trachomatis, a pathogen that typically infects the lower genital tract first, but then if untreated, can also cause damage to ocular surfaces and even cause blindness. Additionally, LL-37 was shown to be significantly induced in the genital tracts of women who were diagnosed positive for C. trachomatis, indicating its importance in natural hos defense. [REF: Chlamydial plasmid-encoded virulence factor Pgp3 interacts with human cathelicidin peptide LL-37 to modulate immune response. Hou S, Sun X, Dong X, Lin H, Tang L, Xue M, Zhong G Microbes Infect 2018 Jun. 26. pii. S1286-4579(18)30142-4. Pubmed ID PMID 29959096] Thus, persons who are sexually active may benefit from maintaining normal to high levels of CAMP gene expression, to increase natural resistance to C. trachomatis infection.

Additionally, for females, sexual activity can often result in chronic urinary tract infections. This can also occur when urinary catheterization is necessary, for medical reasons such as paralysis. Studies have been done demonstrating LL-37's natural ability to prevent infections, for example in the urinary tract epithelium and the urinary bladder. [REF: The antimicrobial peptide cathelicidin protects the urinary tract against invasive bacterial infection. Chromek M, Slamová, Bergman P, Kovács L, Podracká L, Ehren I, Hökfelt T, Gudmundsson H, Gallo R L, Agerberth B, Brauner A. Nat Med 2006 June; 12(6):636-41. Pubmed ID PMID 16751768] and [REF: Vitamin D induction of the human antimicrobial Peptide cathelicidin in the urinary bladder. Hertting O, Holm Å, Luthje P, Brauner H, Dyrdak R, Jonasson A F, Wiklund P, Chromek M, Brauner A. PLoS One 2010 Dec. 14; 5(12):e15580. Pubmed ID PMID 21179490] Maintaining healthy levels of LL-37 expression may reduce the incidence of these infections.

Newborn infants, at birth, are covered in a waxy substance called vernix caseosa, which has potent antimicrobial properties by virtue of its expression of antimicrobial peptides including LL-37. [REF: The newborn infant is protected by an innate antimicrobial barrier: peptide antibiotics are present in the skin and vernix caseosa. Marchini G, Lindow S, Brismar H, Stäbí B, Berggren V, Ulfgren A K, Lonne-Rahm S, Agerberth B, Gudmundsson G H. Br J Dermatol. 2002 December; 147(6). 1127-34. Pubmed ID PMID: 12402861] Thus, LL-37 is an important human host defense peptide, from the earliest mages of life.

### LL-37 Protects Against Stomach Ulcers and Gastric Cancer.

The strain of bacteria that can cause infection of the human stomach lining, Helicobacter pylori or H. pylori, is also susceptible to killing by the cathelicidin peptide LL-37. It was shown that LL-37, either alone or in synergy with another antimicrobial peptide, human beta-defensin 1, was bactericidal for several H. pylori strains. [REF: Expression of LL-37 by human gastric epithelial cells as a potential host defense mechanism against Helicobacter pylori. Hase K, Murakami M, Iimura M, Cole S P, Horibe Y, Ohtake T, Obonyo M, Gallo R L, Eckmann L, Kagnoff M F. Gastroenterology. 2003 December; 125(6):1613-25. Pubmed ID PMID 14724813] Chronic H. pylori infections of the stomach lining can cause ulcers, and also increase susceptibility to stomach cancer. Hence, patients who suffer from H. pylori infection of the stomach lining would likely benefit from an upregulation of the CAMP gene expression of LL-37. Indeed, there is direct evidence that LL-37 expression suppresses the growth of gastric cancer cells [REF: The host defense peptide LL-37 activates the tumor-suppressing bone morphogenetic protean signaling via inhibition of proteasome in gastric cancer cells. Wu W K, Sung J J, To K F, Yu L, Li H T, Li Z J, Chu K M, Yu J, Cho C H. J Cell Physiol. 2010 April, 223(1):178-86. Pubmed ID PMID 20054823]

Complex Associates of LL-37 with Atherosclerosis and Cardiovasular Disease.

In 1998, it was discovered that the human LL-37 peptide binds specifically to the High Density Lipoprotein (HDL)-associated protein Apolipoprotein A-1 (ApoA1); and specifically, that this binding occurs in the context of lipid- and cholesterol-bearing HDL particles, i.e., LL-37 does not bind to ApoA1 in the absence of lipids and cholesterol [REF: Apolipoprotein A-I binds and inhibits the human antibacterial/cytotoxic peptide LL-37. Wang Y, Agerberth B, Löthgren A, Almstedt A, Johansson J. J Biol Chem. 1998 Dec. 11; 273(50):33115-8. Pubmed ID PMID: 9837875] and [REF: The antimicrobial peptide LL-37 binds to the human plasma protein apolipoprotein A-I. Wang Y, Johansson J, Agerberth B, Jörnvall H, Griffiths W J. Rapid Commun Mass Spectrom. 2004; 18(5):588-9. Pubmed ID PMIID 14978905] and [REF: Apoptosis of airway epithelial cells: human serum sensitive induction by the cathelicidin LL-37. Lau Y E, Bowdish D M, Cosseau C, Hancock R E, Davidson D J. Am J Respir Cell Mol Biol. 2006 April; 34(4):399-409. Pubmed D PMID 16340000].

It is significant that LL-37, in the human blood circulation, is found bound to HDL particles, not free in the plasma, because LL-37 peptide, at concentrations above 13 μg/mL, can be cytotoxic to endothelial cells lining blood vessels. However, the normal concentration of LL-37 in human blood (virtually all of which is bound to HDL particles) is 1.15 μg/mL, 11-fold lower than the cytotoxic concentration. [REF: Apolipoprotein A-I attenuates LL-37-induced endothelial cell cytotoxicity. Svensson D, Lagerstedt J O, Nilsson B O, Del Giudice R. Biochem Biophys Res Commun. 2017 Nov. 4; 493(1):71-76. Pubmed ID PMID: 28919413] While its mechanism of involvement is unclear, and LL-37 expression in the cardiovascular system has not been shown to be harmful in human patients, there does appear to be a correlation between cardiovascular disease risk, and CAMP gene expression levels (as measured by mRNA) in blood. [REF: Association of human cathelicidin (hCAP-18/LL-37) gene expression with cardiovascular disease risk factors. Benachour H, Zaiou M, Samara A, Herbeth B, Pfister M, Lambert D, Siest G, Visvikis-Siest S. Nutr Metab Cardiovasc Dis. 2009 December; 19(10):720-8. Pubmed ID PMID 19346112]

Additionally, it has been shown that LL-37 peptide is found in atherosclerotic lesions and plaques. At very high (quite unusually high) concentrations of 30 μg/mL, it can be cytotoxic to endothelial cells. [REF: Human antimicrobial peptide LL-37 is present in atherosclerotic plaques and induces death of vascular smooth muscle cells: a laboratory study. Ciorei C D, Tapper H, Bjartell A, Sternby N H, Bodelsson M. BMC Cardiovasc Disord. 2006 Dec. 20; 6:49. Pubmed ID PMID 17181861] Another, related study also identified the presence of LL-37 in human atherosclerotic lesions, at 6-fold higher concentration than the normal concentration in the blood, and concluded instead that LL-37 acts as a protective immune modulator within atherosclerotic plaques. [REF: Involvement of the antimicrobial peptide LL-37 in human atherosclerosis. Edfeldt K, Agerberth B, Rottenberg M E, Gudmundsson G H, Wang X B, Mandal K, Xu Q, Yan Z Q, Arterioscler Thromb Vase Biol. 2006 July; 26(7):1551-7. Pubmed ID PMID 16645154]

It has further been shown that LL-37 peptide is actively produced within atherosclerotic lesions, being expressed mainly by macrophages within the plaque, but also by the endothelial cells of the blood vessel lining. [REF: Involvement of the antimicrobial peptide LL-37 in human atherosclerosis. Edfeldt K, Agerberth B, Rottenberg M E, Gudmundsson G H, Wang X B, Mandal K, Xu Q, Yan Z Q. Arterioscler Thromb Vasc Biol. 2006 July; 26(7):1551-7. Epub 2006 Apr. 27. Pubmed ID PMID 16645154] On the other hand, studies of the effects of neutrophil-derived LL-37 in the context of injured arteries showed benefit, finding that LL-37 promoted healthy re-endothelialization (heeling of blood vessels), preventing the formation of neointimal hyperplasia (aberrant, scab-like tissues within blood vessels). [REF: Sci Transl Med. 2011 Oct. 5; 3(103):103ra98. Neutrophil-derived cathelicidin protects from neointimal hyperplasia. Soehnlein O, Wantha S, Simsekyilmaz S, Döring Y, Megens R T, Mause S F, Drechsler M, Smeets R, Weinandy S, Schreiber F, Gries T, Jockenhoevel S, Möller M, Vijayan S, van Zandvoort M A, Agerberth B, Pham C T, Galo R L, Hackeng T M, Liehn E A, Zernecke A, Klee D, Weber C. Pubmed ID PMID 21974936]

Taken all together, these data indicate that highly elevated levels of LL-37 expression in the cardiovascular system (greater than 10× normal concentrations) can have cytotoxic effects on the human circulatory system. However, it is also worth noting that, while the benefits of cathelicidin expression are clear in the context of preventing numerous types of infections of bacterial, fungal, or viral origin, it is not the case that highly elevated concentrations will be beneficial in every circumstance. Hence, monitoring of cathelicidin expression levels within the blood, to ensure that it is less than 6× the normal concentration, would be beneficial.

### The Role of LL-37 in Cancer is Complex.

The cathelicidin peptide has also been studied in the context of cancer. Initially, it was shown that, if the mouse gene for cathelicidin (camp) was knocked out, creating a camp (−/−) mouse, that transgenic mouse was much less able to fight cancer in an orthotopic tumor cell injection model, specifically because the mouse's Natural Killer (NK) cells did not express the cathelicidin peptide as an anticancer molecule. [REF: The host defense peptide cathelicidin is required for NK cell-mediated suppression of tumor growth. Büchau A S, Morizane S, Trowbridge J, Schauber J, Kotol P, Bue J D, Gallo R L. J Immunol. 2010 Jan. 1; 184(1):369-78. Pubmed ID PMID 19949065] It has also been shown, in the context of B cell lymphoma (a blood cell cancer), that Vitamin D3-induced expression of cathelicidin results in beneficial cytotoxic activity of M1 inflammatory macrophages against high-grade B cell lymphoma. [REF: Vitamin D-dependent induction of cathelicidin in human macrophages results in cytotoxicity against high-grade B cell lymphoma. Bruns H, Büttner M, Fabri M, Mougiakakos D, Bittenbring J T, Hoffmann M H, Beier F, Pasemann S, Jitschin A, Hofmann A D, Neumann F, Daniel C, Maurberger A, Kempkes B, Amann K, Mackensen A, Gerbitz A. Sci Transl Med. 2015 Apr. 8; 7(282):282ra47. Pubmed ID PMID 25855493] The mechanism by which the cathelicidin peptide can exert anti-cancers on Jurkat T-cell lymphoma also has been studied, and LL-37 was shown to induce cancer cell death in a calpain- and apoptosis-inducing factor (AIF) dependent manner involving Bax activity. [REF: The human host defense peptide LL-37 induces apoptosis in a calpain- and apoptosis-inducing factor-dependent manner involving Bax activity. Mol Cancer Res 2009 May; 7(5):689-702. Mader J S, Mookherjee N, Hancock R E, Bleackley R C. Pubmed ID PMID 19435812]

Another study found that LL-37 can suppress cancer growth in gastric adenocarcinoma (stomach cancer) cells, by activating a tumor-suppressing gene BMP (bone morphogenic protein) signaling pathway. [REF: The host defense peptide LL-37 activates the tumor-suppressing bone morphogenic protein signaling via inhibition of proteasome in gastric cancer cells. J Cell Physiol. 2010 April; 223(1); 178-86. Wu W K, Sung J J, To K F, Yu L, Li H T, Li Z J, Chu K M, Yu J, Cho C H.] However, whereas LL-37 shows beneficial anticancer activities for lymphomas, in some tissue-based cancers involving solid tumors, the results of studies are mixed, and seem to indicate that LL-37 expression is upregulated in the tumor microenvironment and may promote metastasis, acting as a growth factor for epithelial cell-type cancers (which makes sense, given its natural involvement in wound healing and angiogenesis). [REF: Antimicrobial protein hCAP18/LL-37 is highly expressed in breast cancer and is a putative growth factor for epithelial cells Heilborn J D, Nilsson M F, Jimenez C I, Sandstedt B, Borregaard N, Tham E, Sørensen O E, Weber G, Stáhle M. Int J Cancer. 2005 May 1; 114(5):713-9. Pubmed ID PMID 15609314]

What is unclear, is whether this is a consequence of the tumor, a byproduct of the tumor, or a pathogenic element of solid tumors. In the case of ovarian cancer, LL-37 is overexpressed and appears to play a negative role, increasing ovarian cancer cell proliferation and invasion. [REF: Ovarian cancers overexpress the antimicrobial protein hCAP-18 and its derivative LL-37 increases ovarian cancer cell proliferation and invasion. Coffelt S B, Waterman R S, Florez L, Höner zu Bentrup K, Zwezdaryk K J, Tomchuck S L, LaMarca H L, Danka E S, Morris C A, Scandurro A B. Int J Cancer. 2008 Mar. 1; 122(5):1030-9. Pubmed ID PMID 17960624] A negative role for LL-37 also has been found in the case of malignant melanoma. [REF: The antimicrobial peptide human cationic antimicrobial protein-18/cathelicidin LL-37 as a putative growth factor for malignant melanoma. Kim J E, Kim H J, Choi J M, Lee K H, Kim T Y, Cho B K, Jung J Y, Chung K Y, Cho D, Park H J. Br J Dermatol. 2010 November; 163(5):959-67. Pubmed ID PMID 20977442]

Based on this literature, speaking broadly, it is not yet clear whether LL-37 expression plays a positive, anticancer role or negative, pro-metastasis role in cancer. It appears to be beneficial to upregulate LL-37 expression in the context of blood cancers like B cell lymphoma and Jurkat T cell lymphoma. On the other hand, in cases of breast cancer, malignant melanoma, and ovarian cancer, increasing LL-37 concentrations may not be beneficial.

### SUMMARY OF THE DISCLOSURE

In one aspect, a method is provided for upregulating cathelicidin gene expression in the brain of a subject. The method comprises forming a pharmaceutically acceptable composition which includes a mixture of materials selected from the group consisting of Vitamin D3, phenylbutyrate, bexarotene, DHA, curcumin, resveratrol, and pharmaceutically acceptable salts thereof; and administering the pharmaceutically acceptable composition to the subject.

In another aspect, a method is provided for treating a human subject for Alzheimer's disease. The method comprises diagnosing the human subject as suffering from Alzheimer's disease, and applying to the subject a pharmaceutically acceptable composition which upregulates cathelicidin gene expression in the brain of the subject.

In a further aspect, a method is provided for treating β-amyloid aggregation and accumulation in a subject. The method comprises detecting the presence of β-amyloid aggregate accumulation in tissues of the subject; and administering to the subject a pharmaceutically acceptable composition which upregulates cathelicidin gene expression in the tissues of the subject.

In yet another aspect, a method is provided for treating a subject. The method comprises monitoring levels of the cathelicidin peptide LL-37 and β-amyloid in tissues of a subject; and, when the condition L/B<k is detected, where L is the level of LL-37 detected, B is the level of β-amyloid detected, and k is a predetermined threshold value, upregulating cathelicidin gene expression in the tissues of the subject.

In still another aspect, a method is provided for modulating in vivo β-amyloid (Aβ) fibril formation. The method comprises monitoring the level of β-amyloid in tissues of a subject; and administering to the subject a pharmaceutically active composition which modulates in vivo fibril formation in said tissues by inducing the expression of a physiologically effective binding partner for β-amyloid.

In another aspect, a method is provided for modulating in vivo fibril formation. The method comprises co-incubating β-amyloid (Aβ) with a physiologically effective binding partner for β-amyloid, thereby obtaining co-incubated peptides; creating a pharmaceutical composition from the co-incubated polypeptides; and administering the pharmaceutical composition to a subject.

In a further aspect, a method for modulating microglia-mediated neuroinflammation is provided. The method comprises monitoring the level of a cytokine in microglia tissues of a subject, wherein the cytokine is selected from the group consisting of TNFα and IL-6; and administering to the subject a pharmaceutically active composition which reduces the level of the cytokine in the microglia tissues of the subject, wherein the pharmaceutically active composition induces the expression of a physiologically effective binding partner for β-amyloid in the microglia tissues of the subject.

In another aspect, a method is provided for inhibiting in vivo β-amyloid (Aβ) fibril formation in the tissues of a subject in which an equilibrium exists between smaller and larger MW species of β-amyloid (Aβ). The method comprises administering to the subject a pharmaceutical composition which shifts the equilibrium toward the smaller species of Aβ.

In yet another aspect, a method is provided for modulating in vivo β-amyloid (Aβ) fibril formation. The method comprises monitoring the level of β-amyloid in tissues of a subject, determining that the monitored level of β-amyloid has exceeded a predetermined threshold; and reducing the level of β-amyloid in the tissues below the threshold amount by inducing LL-37 production in the tissues through the stimulation of glial cells with a stimulant selected from the group consisting of lipopolysaccharide/interferon-gamma (LPS/IFNγ) and IFNγ.

### BRIEF DESCRIPTION OF THE DRAWINGS

FIGS. 1-3 are a series of CE electropherograms. FIG. 1 is for Aβ₄₀ (t=0 days), FIG. 2 is for Aβ₄₂ (t=0 days), and FIG. 3 is for Aβ₄₂ (t=24 days). In the CE traces, the designation ● refers to low MW-oligomers, and the designation * refers to high MW-oligomers.

FIG. 2. 4-9 depict the results of binding studies performed by SPRi. The SPRi-chip was functionalized with Aβ₄₀ (t=0 days) (FIGS. 4 and 7); Aβ₄₂ (t=0 days) (FIGS. 5 and 8), and Aβ₄₂ (t=24 days) (FIGS. 6 and 9). All the Aβ peptides were immobilized in replicate (n=8) on the same SPRi chip at the same concentration (20 μM). SPRi reference-corrected responses related to LL-37 (10 μM)(black) and ovalbumin (10 μM) (gray) (negative control) flowed on the SPRi-chip functionalized with different Aβ forms (FIGS. 4-6). The three SPRi sensograms show the injection of running buffer (baseline) (1), the injection of the analyte (association phase) (2) and the subsequent injection of buffer (dissociation phase) (3). Calibration curve of LL-37 flowed onto different Aβ forms immobilized on the SPRi-chip Aβ₄₀ (t=0 days, FIG. 7), A42 (t=0 days, FIG. 8), Aβ₄₂ (t=24 days, FIG. 9). The equilibrium binding constants (K_{A} and K_{D}) values were calculated using a nonlinear curve fit of the SPRi response at equilibrium (see FIG. 9).

FIGS. 10-14 are Transmission Electron Microscopy (TEM) images for 50 μM Aβ₄₂ at t=0 (FIG. 10); 100 μM LL-37 at t=10 days (FIG. 11); equimolar mixtures of 50 μM Aβ₄₂ and LL-37 at t=0 (FIG. 32); t=3 days (FIG. 13); and t=9 days (FIG. 14). Scale bar: 200 nm, magnification 60,000×.

FIGS. 15-16 are circular dichroism spectra of peptide solutions, recorded at t=0 min (FIG. 15) and t=24 hours (FIG. 16). Blue line: 50 μM Aβ₄₂; red line: 50 μM LL-37; green line: 1:1 mixture.

FIG. 17 is a listing of amino acid sequences for human cathelicidin peptide LL-37 (so named because it comprises 37 amino acids) and the human amyloid-β peptide. Aβ₄₂.

FIG. 18-20 are graphs depicting the effects of treatment with Aβ, LL-37, or their mixture on the viability changes of SH-SY5Y cells induced by microglial-mediated toxicity in 72 h (FIG. 13) and levels of cytokines, TNFα (FIG. 19) and IL-6 (FIG. 20) in microglial toxic supernatant. Values are mean±SEM, n=4. One-way ANOVA was carried out to test significance. Multiple group comparisons were followed by a post-hoc Bonferroni test where necessary. *P<0.01 for Aβ-treated cells and LL-37-exposed cells compared with control (CON) group and **P<0.01 for Aβ-LL-37-treated groups compared with Aβ-treated cells and LL-37-exposed cells. Note that Aβ and LL-37 inhibit the microglial activation of each other.

FIG. 21 is a series of plasmonic curves acquired before the injection of LL-37 peptide. The shift between these curves is related to the amount of molecules immobilized on the SPRi chip surface. In particular, the plasmonic curves related to both Aβ₄₀ (t=0 days) and Aβ₄₂ (t=24 days) show a significant shift from that related to Aβ₄₀ solubilized in PBS (t=0) and from that of the reference peptide (negative) and to the chip (no molecules adsorbed)

FIG. 22 is a CCD image of the chip surface. The size of spots measured directly by this type of images was around 190 μm.

FIGS. 23-25 are reference-corrected responses related to seven dilutions of LL-37 injected on the SPRi-chip functionalized with (FIG. 23) Aβ40) solubilized in PBS; (FIG. 24) Aβ42 t=0 days and (FIG. 25) Aβ42 t=24 days, used to calculate the equilibrium binding constants (K_{D}) in FIG. 4. All the responses reached the equilibrium (plateau) before the end of the injection (500 s)

FIG. 26 is a CCD differential image of the SPRi-biochip during the late association phase (500 s) of LL-37 injected at concentration 25 μM. “Ref” is the reference peptide used as negative control on the chip surface.

FIGS. 27-29 depict the results of an equilibrium-binding analysis of the control scrambled LL-37 peptide, performed by surface plasmon resonance imaging (SPRi) using the same procedures and conditions used to study LL-37 (compare with FIG. 16). The SPRi-chip was functionalized with Aβ40 (t=0 days) (FIG. 27); Aβ42 (t=0 days)(FIG. 28) and Aβ42 (t=24 days) (FIG. 29). The equilibrium binding constants and the fitting curves could not be calculated, due to the irregular and negative dose-response trend.

FIG. 30 is a graph depicting SH-SY5Y, Microglia and SH-SY5Y/Microglia viability changes induced by Aβ peptide and LL-37 peptide (30 μM each). Reductions in numbers of live cells are indicated by the MTT assay. Values are mean±SEM, n=4. One-way ANOVA was carried out to test significance. Multiple group comparisons were followed by a post-hoc Bonferroni test where necessary. *P<0.01 for cells in 72 h compared with the ones in 0 h and **P<0.01 for SH-SY5Y plus microglia without Aβ or LL-37 peptides group compared with SH-SY5Y cells or microglial cells in the same incubation time. Note that the cell viability of SH-SY5Y and microglia co-culture is nearly equal to the sum of two cell culture group in 72 h, and that treatment with either Aβ or LL-37 alone did not change viability of either cell type (alone) over the course of 72 h.

FIG. 31 is a graph depicting the results of induction of the camp (cathelicidin) gene in the brain of wild type c57b/6j mice treated for 2 weeks with oral polytherapy. Mice also had access to a running wheel.

FIGS. 32-34 are a series of graphs depicting the effects of treatment on 5XFAD model mice vs. untreated 5XFAD mice and wild type C57BL/6J mice, and vs. treated C57BL/6J mice. FIG. 32 depicts the results for contextual memory total (5 min), FIG. 33 depicts the results far contextual memory total excluding the first minute, and FIG. 34 depicts contextual memory at each minute of the trial. In Contextual Fear Conditioning testing (72 hours), which tests memory and recall, treated 5XFAD mice show an improved memory of contextual fear, as compared to untreated 5XFAD mice. Treated 5XFAD mice show memory comparable to wild type mice.

FIGS. 35-6 depict the chemical structures of orally delivered cathelicidin gene inducers.

### DETAILED DESCRIPTION

It will be appreciated from the literature review and discussion presented in the Background Section that the protein product of the CAMP gene, the innate immune effector LL-37, has been studied extensively in the context of infectious disease. LL-37 has also been studied, to a lesser degree, in the context of wound healing, angiogenesis and stem cells, and cancer. However, it has now been discovered that a direct relation exists between LL-37 and Alzheimer's Disease. See [REF: Evidence that the Human Innate Immune Peptide LL-37 may be a Binding Partner of Amyloid-β and Inhibitor of Fibril Assembly De Lorenzi E, Chiari M, Colombo R, Cretich M, Sola L, Vanna R, Gagni P, Bisceglia F, Morasso C, Lin J S, Lee M, McGeer P L, Barron A E. J Alzheimers Dis. 2017; 59(4):1213-1226. Pubmed ID PMID 28731438], which is incorporated by reference in its entirety. This surprising discovery has not been appreciated in the relevant art, and led to the further discovery by the present inventors that Alzheimer's Disease may be treated by inducing expression of the CAMP gene encoding the LL-37 peptide. This discovery is a key technical foundation of the methodologies disclosed herein.

### Cathelicidin LL-37 in the Context of Alzheimer's Disease.

Alzheimer's Disease is a terminal diagnosis suffered by over 42 million patients globally, with no effective therapy or treatment. The handful of drugs currently prescribed to Alzheimer's patients at best ameliorate symptoms (‘disease-modifying’ as opposed to ‘disease-curative’ treatments). Most ‘symptomatic therapies’ currently available are neurotransmitter-focused, and fall into two classes: cholinesterase inhibitors, and the NMDA (glutamatergic) receptor agonist memantine. There are no FDA-approved drugs that address the underlying causes of Alzheimer's disease, which remain poorly understood despite 110 years of research. [REF: Alzheimer's Disease: Lessons Learned from Amyloidocentric Clinical Trials. Soejitno A, Tjan A, Purwata T E, CNS Drugs. 2015 June; 29(6):487-502. PMID: 26187557] Alzheimer's disease risk is known to correlate with the risks of several other chronic diseases, in particular metabolic syndrome/type 2 diabetes and cardiovascular disease. However, these connections are understudied, given the complexity of studying such associations. In practice, pharmaceutical drug development is typically undertaken for only one chronic disease target at a time, although some FDA-approved drugs are repurposed for other conditions.

As of 2018, it has been 14 years since a new drug for Alzheimer's disease was approved by FDA. The majority of the more than 400+ failed clinical trials pursued strategies to reduce the distinct, observable pathophysiological protein signatures of Alzheimer's disease. These protein signatures include Aβ amyloid plaque accumulation in the brain, which is primarily extracellular (outside of neurons), and (much less commonly) phosphorylated tau protein aggregates, which form ‘neurofibrillary tangles’ within neuronal cells. Clinical trials aimed at reducing the burden of Aβ and tau aggregate protein deposits have so far failed to improve patient cognition and function, as was required by the FDA for drug approval (until recently, when FDA relaxed these criteria somewhat) [REF: Prevention of sporadic Alzheimer's disease: lessons learned from clinical trials and future directions. Andrieu S, Coley N, Lovestone S, Aisen P S, Vellas B Lancet Neurol. 2015 September: 14(9):926-944. PMID: 26213339].

Within the aging human brain, Alzheimer's disease (AD) involves the assembly of β-amyloid (Aβ) peptides from soluble monomers into oligomers, fibrils and plaques [REF: Alzheimer mechanisms and therapeutic strategies. Huang Y, Mucke L. Cell. 2012 Mar. 16; 148(6):1204-22. Review. Pubmed ID PMID: 22424230]. Studies of the spatiotemporal interplay between diffusible Aβ oligomers and fibrillar deposits, as well as intracellular tau tangles, have been aimed at investigating drivers of neuronal dysfunction, which are still not well understood [REF: Alzheimer's and Parkinson's diseases. The prion concept in relation to assembled Aβ, tau, and α-synuclein. Goedert M. Science. 2015 Aug. 7; 349(6248):1255555. Pubmed PMID 26250687] and [REF: Alzheimer's Disease: Lessons Learned from Amyloidocentric Clinical Trials. Soejitno A, Tjan A, Purwata T E. CNS Drugs. 2015 June; 29(6):487-502. doi: 10.1007/s40263-015-0257-8. Pubmed ID PMID 26187557] The root physiological causes of sporadic (i.e., spontaneously arising) Alzheimer's Disease—i.e., instances of disease in patients for whom there is no known genetic predisposition, which is the most common type of AD-remain unspecified, preventing the development of effective therapies to prevent, halt, or reverse the disease [REP: Prevention of sporadic Alzheimer's disease: lessons learned from clinical trials and future directions. Andrieu S, Coley N, Lovestone S, Aisen P S, Vellas B. Lancet Neurol. 2015 September; 14(9):926-944. Pubmed ID PMID 26213339].

Identification of physiologically relevant binding partners of Aβ that modulate fibril formation in vivo may yield new insights into causes of AD and help identify early instigators of Aβ accumulation and neurotoxic effects. A number of natural proteins that can bind to, or interact with, Aβ have been identified by various assays [REF: Molecules that target beta-amyloid. Stains C I, Mondal K, Ghosh I, ChemMedChem. 2007 December; 2(12):1474-92 Review. Pubmed ID PMID: 17952881]. However, their roles in sporadic AD are often challenging to assess.

Literature reports exist which might arguably be interpreted to suggest, in an indirect, “connect-the-dots” fashion, that the biophysical activities and signaling functions of Aβ peptides and LL-37 (the only cathelicidin-derived innate immune system peptide found in humans) are related in vivo. For instance, the vitamin D receptor (VDR) and retinoid X receptor (RXR) am both connected with AD, as well as with expression levels of LL-37. Vitamin D3 treatment was shown to reduce cerebral amyloid-β accumulation and to improve cognition in a mouse model of AD, although the mechanism of action was not stated [REF: 1α,25-Dihydroxyvitamin D3 reduces cerebral amyloid-β accumulation and improves cognition in mouse models of Alzheimer's disease. Durk M R, Han K, Chow E C, Ahrens R, Henderson J T, Fraser P E, Pang K S, J Neurosci. 2014 May 21; 34(21).7091-101 doi. 10.1523/JNEUROSCI.2711-13.2014. Pubmed ID PMID 24849345], while RXR activation reduced neuronal loss and improved cognition in an aggressive mouse model of AD (but again, with no mention of cathelicidin involvement) [REF: Neuronally directed effects of RXR activation in a mouse model of Alzheimer's disease. Mariani M M, Malm T, Lamb R, Jay T R, Neilson L, Casali B, Medarametla L, Landreth G E. Sci Rep. 2017 Feb. 16; 7:42270. Pubmed ID PMID 28205585]. The above AD papers do not mention that expression levels of the human CAMP gene that encodes hCAP-18 (the protein precursor for LL-37) are upregulated by activation of VDR. [REF: The chemistry and biology of LL-37. Burton M F, Steel P G. Nat Prod Rep. 2009 December; 26(12).1572-44. Review. PMID 19936387] and [REF: Human cathelicidin antimicrobial peptide (CAMP) gene is a direct target of the vitamin D receptor and is strongly up-regulated in myeloid cells by 1,25-dihydroxyvitamin D3 Gombart A F, Borregaard N, Koeffler H P. FASEB J. 2005 July; 19(9):1167-77. Pubmed ID PMID 15985530] These references also do not mention that RXR activation is an obligate part of CAMP gene expression, although this has been shown. [REF: Label-free quantitative mass spectrometry reveals novel pathways involved in LL-37 expression. Cederlund A, Nylén F, Miraglia E, Bergman P, Gudmundsson G H, Agerberth B. J Innate Immun. 2014; 6(3):365-76. Pubmed ID PMID 24246949] and [REF: Cell Tissue Res. 2016 November; 366(2):353-362. Vitamin D-induced up-regulation of human keratinocyte cathelicidin anti-microbial peptide expression involves retinoid X receptor α. Svensson D, Nebel D, Voss U, Ekblad E, Nilsson B O. Pubmed ID PMID 27357804]

Another indirect connection between Alzheimer's Disease mechanism and cathelicidin LL-37 is the hypothesis that sporadic AD is essentially ‘Type 3 Diabetes’ occurring in brain tissue [REF: Type 3 diabetes is sporadic Alzheimers disease: mini-review, de la Monte S M. Eur Neuropsychopharmacol. 2014 December; 24(12):194-60. Pubmed ID PMID 25088942] and [REF: Alzheimer's disease is type 3 diabetes-evidence reviewed. de la Monte S M, Wands J R. J Diabetes Sci Technol. 2008 November; 2(6):1101-13. Pubmed ID PMID 19885299]. In a 2015 study, it was shown that intraperitoneal administration of the murine cathelicidin peptide, CRAMP (which, like LL-37 for humans, is unique in the mouse proteome) protects Non-Obese Diabetic (NOD) mice against the development of autoimmune diabetes. [REF: Pancreatic β-Cells Limit Autoimmune Diabetes via an Immunoregulatory Antimicrobial Peptide Expressed under the Influence of the Gut Microbiota. Sun J, Furio L, Mecheri R, van der Does A M, Lundeberg E, Saveanu L, Chen Y, van Endert P, Agerberth B, Diana J. Immunity. 2015 Aug. 18; 43(2):304-17. Pubmed ID PMID 26253786] This treatment was motivated by the authors' discovery that the genetic defect of NOD mice that creates susceptibility to autoimmune diabetes is a deficit in their ability to express cathelicidin. This defect is more pronounced in female NOD mice, who have a higher incidence of disease as compared with males. In this animal model, immunomodulatory effects of the cathelicidin peptide, including effects on the phenotypes of white blood cells including macrophages, dendritic cells, T and B cells, reduced risks of inflammatory disease [REF: Pancreatic β-Cells Limit Autoimmune Diabetes via an immunoregulatory Antimicrobial Peptide Expressed under the influence of the Gut Microbiota Sun J, Furio L, Mecheri R, van der Does A M, Lundeberg E, Saveanu L, Chen Y, van Endert P, Agerberth B, Diana J. Immunity. 2015 Aug. 18; 43(2):304-17. Pubmed ID PMID 26253786]. What this shows is that, in vivo, the cathelicidin peptide is strongly immunomodulatory of white blood cell phenotypes. Thus, macrophages, dendritic cells, T cells, and B cells, were all switched from a proinflammatory, diabetogenic phenotype by the injection of cathelicidin peptide, to a noninflammatory, healing phenotype that prevented the development of autoimmune diabetes.

To date, most approaches to AD have relied on the supposition that pathological overexpression or hindered degradation of Aβ lays the primary foundation for disease. [REF: Alzheimer mechanisms and therapeutic strategies. Huang V, Mucke L. Cell. 212 Mar. 16; 148(6).1204-22 Review. Pubmed ID PMID: 22424230]. Recently, it has been shown that the chronic under-expression of the cathelicidin LL-37, which normally opposes Aβ fibril formation, likely plays a key role in the pathological accumulation of Aβ, as will be discussed below [REF: Evidence that the Human Innate Immune Peptide LL-37 may be a Binding Partner of Amyloid-β and Inhibitor of Fibril Assembly. De Lorenzi E, Chiari M, Colombo R, Cretich M, Sola L, Vanna R, Gagni P, Bisceglia F, Morass C, Lin J S, Lee M, McGeer P L, Barron A E. J Alzheimers Dis. 2017; 59(4).1213-1226. Pubmed ID PMID 29731438]. It is, of course, difficult for researchers to identify a systemic element that should be present, or perhaps should be better regulated, but is not. This may explain why the central and important role of LL-37 under-expression in Alzheimer's Disease was not discovered prior to 2017.

Recently, a multifaceted approach was taken to confirm and characterize, in vitro, the interactions between Aβ and LL-37, and inhibitory effects of LL-37 on Aβ oligomer/fibril formation. It was demonstrated that LL-37 and Aβ42 (residues 1-42 of the Aβ peptide), which am both individually toxic and proinflammatory to neuroblastoma cell line SH-SY5Y via the stimulation of microglial production of inflammatory cytokines, lose most of their cytotoxicity to neurons if the two peptides are co-incubated prior to being added to the cell culture media.

The interactions between LL-37 and Aβ peptides were investigated by SPR imaging (SPRi), a recent evolution of traditional SPR, which couples the label-free monitoring of molecular interactions by scanning angle SPR measurements with simultaneous CCD-based imaging of the whole surface for signal detection [REF: Surface plasmon resonance imaging for affinity-based biosensors. Scarano S, Mascini M, Turner A P, Minunni M. Biosens Bioelectron. 2010 Jan. 1; 25(5):957-66. Pubmed ID PMID 19765967]. The multi-array SPRi configuration improves the overall accuracy of the study by allowing simultaneous detection of signals originating from both positive and negative control ligands immobilized on the same chip. In addition, the real-time imaging of the entire SPR-biochip allows for verification of the quality and the optical properties of different ligands after their immobilization on the chip surface. To evaluate the aggregation state and the presence of soluble oligomers in the AP samples used for SPRi, capillary electrophoresis (CE) analysis was carried out. The inhibitory effect on fibril formation was demonstrated through transmission electron microscopy (TEM) by investigating fibril formation in quasi-physiological conditions. Conformational analyses of Aβ42 peptide in solution, in the absence and presence of LL-37, were cared out by circular dichroism (CD) spectroscopy.

In this work [REF: Evidence that the Human Innate Immune Peptide LL-37 may be a Binding Partner of Amyloid-β and Inhibitor of Fibril Assembly. De Lorenzi E, Chai M, Colombo R, Cretich M, Sola L, Vanna R, Gagni P, Bisceglia F, Morasso C, Lin J S, Lee M, McGeer P L, Barron A E. J Alzheimers Dis 2017; 59(4):1213-1226. Pubmed ID PMID 28731438], it was demonstrated that Aβ and LL-37 bind to each other specifically, and that LL-37 inhibits the adoption by Aβ of ordered β-type secondary structures. Additionally, a variety of different published findings were discussed that indicate a physiological interplay between (and potential co-regulation of) these two peptides as an aspect of human innate immunity that may affect the initiation and progression of Alzheimer's Disease-related pathology.

### Materials and Methodologies

The methodologies disclosed herein may be further appreciated with respect to the following non-limiting examples. The following materials and methodologies were utilized in the examples provided herein.

### Sourcing Peptides and Buffer Chemicals; Materials and Methods for Sample Preparation

Aβ40, Aβ42 free base peptides and 1% NH4OH can be purchased from AnaSpec (Fremont Calif., USA). LL-37 was from Innovagen (Lund, Sweden). An unrelated peptide to serve as a binding study control (sequence SYSVQDQYQALLQQHAQYK) was kindly gifted by Dr. Alessandro Gori from the Peptide Synthesis Lab, ICRM, CNR, Milan. “Scrambled sequence” LL-37 peptide was from AnaSpec, and has the same amino acid composition as natural LL-37, but with the following sequence: GLKLRFEFSKIKGEFLKTPEVRFRDIKLKDNRISVQR. 1,1,1,3,3,3-Hexafluoro-2-propanol (HFIP), acetonitrile, sodium carbonate, sodium hydroxide as well as NaH2PO4 and Na2HPO4 were from Sigma-Aldrich (Stenheim, Germany). Buffer solutions were prepared daily using Millipore Direct-Q™-deionized water, filtered with 0.45 μm Millipore membrane filters (Bedford, Mass., USA) and degassed by sonication.

### Samples and Methods Used for SPRi and CE Experiments

In SPRi and CE experiments, 1 mg/mL stock solutions of Aβ40 and unrelated peptide or scrambled-sequence LL-37 in PBS were diluted to the desired concentration. Aβ42 was prepared as described by Bartolini et al. [REF: Kinetic characterization of amyloid-beta 1-42 aggregation with a multimethodological approach. Bartolini M, Naldi M, Fiori J. Valle F, Biscarini F, Nicolau D V, Andrisano V. Anal Biochem. 2011 Jul. 15; 414(2):215-25. Pubmed ID PMID 21435333]. Briefly, 0.5 mg of lyophilized Aβ42 powder was dissolved in HFIP (149 μM) and kept overnight at room temperature. After 12 hours, the solution was aliquoted and the HFIP was left to evaporate overnight at room temperature. The Aβ42 film was redissolved in CH3CN/300 μM Na2CO3/250 mM NaOH (48.3:48.3:3.4, v/v/v). The obtained 500 μM Aβ42 solution was diluted to the desired concentration with 20 mM phosphate buffer, pH 7.4 (SPRi: 20 μM; CE: 100 μM).

### Methods for TEM and CE Analysis

For TEM and CD analysis, the samples were prepared as described in Brogi et al. [REF: Disease-modifying anti-Alzheimer's drugs: inhibitors of human cholinesterases interfering with β-amyloid aggregation. Brogi S, Butini S, Maramai S, Colombo R, Verga L, Lanni C, De Lorenzi E, Lamponi S, Andreassi M, Bartolini M, Andrisano V, Novellino E, Campiani G, Brindisi M, Gemma S. CNS Neurosci Ther. 2014 July, 20(7):624-32. Pubmed ID PMID 24935788], and analyzed at 50 and/or 100 μM concentrations. Briefly, to 1 mg lyophilized Aβ42 powder (AnaSpec) 80 L of 1% NH₄OH solution was added. After 1 minute sonication, the solution was diluted with 20 mM phosphate buffer, pH 7.4, to the desired concentration, and divided into aliquots, which were freeze-dried and stored at −80° C. Each lyophilized peptide aliquot was resuspended in 20 mM phosphate buffer, pH 7.4 immediately before use. Samples were sonicated 3 min in an ultrasonic bath, to break down pre-formed aggregates and increase the peptide's effective concentration [REF: Sonication of proteins causes formation of aggregates that resemble amyloid. Stathopulos P B, Scholz G A, Hwang Y M, Rumfeldt J A, Lepock J R, Meiering E M, Protein Sci. 2004 November; 13(11):3017-27. Pubmed ID PMID 15459333]. For samples incubated with LL-37, lyophilized peptide was re-suspended in appropriately diluted LL-37 solutions, so as to keep the peptides at the desired molar concentrations and obtain proper Aβ42/LL-37 molar ratios. Samples were analyzed either immediately after solubilization, or after different lengths of incubation time, as noted in FIG. legends.

Methods for Surface Plasmon Resonance Imaging (SPRi) Experiments with LL-37 and Aβ

Bare gold SPRi-biochips (HORIBA Jobin Yvon SAS, France) were treated for 10 minutes with an Oxygen Plasma Generator (Harrick Plasma Cleaner), then coated using a solution of co-poly(DMA-NAS-MAPS) (MCP-2, Lucidant Polymers, Sunnyvale, Calif., USA) via surface immersion for 30 min (MCP-2 copolymer was dissolved at 1% w/v in aqueous 20% ammonium-sulfate). Coated SPRi-biochips were then washed with distilled water and dried at 80° C. for 15 minutes. Residual reactive groups were then reacted with 50 mM ethanolamine/0.1M Tris pH 9.0 for 1 hour, washed with water, and dried with nitrogen.

The SPRi-biochip surface coated with the copolymer was functionalized by spotting the following 20 μM solutions: 1) Aβ40 (t=0 days); 2) Aβ42 (t=0 days); 3) Aβ42 (t=24 days, i.e. spotted after 24 days of incubation at room temperature) and 4) an unrelated peptide used as reference. These peptides were spotted in replicate (8 spots, around 190 μm diameter each) using a piezoelectric spotter (SciFLEXARAYER S5; Scienion) at 20° C. and 50% humidity. After spotting, the SPRi-biochip was incubated overnight in a sealed chamber saturated with sodium chloride at room temperature.

The binding analysis was performed using a Horiba XelPeX SPRi imaging instrumentation (HORIBA Jobin Yvon SAS, France) after an overnight instrument equilibration with HBS-T (10 mM HEPES, 150 mM NaCl, 0.05% Tween) used also as running buffer. Thereafter increasing concentrations of LL-37 peptide solution (1.0, 2.5, 5.0, 10, 25, 50, 100 μM in phosphate buffer 20 mM, pH 7.4) were injected onto the chip at 50 μL/min for 10 minutes, at 25° C., in order to reach a binding equilibrium for all the responses. Ovalbumin (Sigma-Aldrich, A5503) was used as the negative control sample. After each injection, the chip surface was regenerated by injecting 50 mM glycine at pH 2.0, 50 μL/min for 4 minutes, thus obtaining a complete regeneration of the chip surface without significant loss of binding capacity. The results were preprocessed using EzSuite (HORIBA Jobin Yvon SAS, France), then equilibrium-binding constants (KD and KA (1/KD) values were calculated using Scrubbergen2 (licensed by HORIBA Jobin Yvon SAS). Additional binding analysis experiments were performed using the same procedures and conditions, but by injecting a control scrambled-sequence LL-37 peptide.

### Methods for Capillary Electrophoresis (CE) Analysis of A/β Peptides

All CE experiments were carried out by slightly modifying the method reported by Brogi et al. [REF: Disease-modifying anti-Alzheimer's drugs: inhibitors of human cholinesterases interfering with β-amyloid aggregation. Brogi S, Butini S, Maramai S, Colombo R, Verga L, Lanni C, De Lorenzi E, Lamponi S, Andreassi M, Bartolini M, Andrisano V, Novellino E, Campiani G, Brindisi M, Gemma S. CNS Neurosci Ther. 2014 July; 20(7):624-32. Pubmed ID PMID 24935788). Briefly, an Agilent Technologies 3D CE system with built-in diode-array detector (Waldbronn, Germany) was used and data were collected using Chemstation A.10.02 software. The fused-silica capillary (50 μm id, 360 μm od, total length 33 cm, effective length 24.5 cm) was from Polymicro Technologies (Phoenix, Ariz., USA). A new capillary was flushed at 1 bar with l M NaOH and water for 60 min each and background electrolyte (BGE, 80 mM sodium phosphate buffer, pH 7.4) for 90 min. The between-run rinsing cycle consisted of 50 mM SDS (1.5 min), water (1.5 min) and BGE (2 min). Sample injection (100 μM) was carried out by applying a pressure of 30 mbar for 3 s. Separations were carried out at 25° C. and +12 kV (current 75-80 μA). The acquisition wavelength was 200 nm.

### Methods for Transmission Electron Microscopy (TEM) Analysis of Aβ and LL-37 Peptides

For each TEM sample, a 10-μL droplet of suspension or solution was applied to a carbon-coated Formvar nickel grid (200 mesh) (Electron Microscopy Sciences, Washington, Pa. USA). Each sample was allowed to sediment onto the carbon film for 15 min, then negative staining was performed with 10 μL of 2% w/v uranyl acetate solution (Electron Microscopy Sciences). After carefully draining off excess staining solution using filter paper, the specimen was transferred to a Philips CM12 transmission electron microscope for examination, operating the TEM at 80 kV. Electron micrographs of negatively stained samples were photographed using Kodak film.

### Methods for Circular Dichroism Analysis of Aβ and LL-37 Peptides

CD spectra were collected at 20° C. using a Jasco-810 spectrophotometer and a quartz cuvette with a 0.1 cm path length. All experiments were performed with an Aβ42 concentration of 50 μM. Spectra were registered from 190 to 250 nm and run at a scan speed of 20 nm/min, with a time response of 2 sec and data pitch of 0.2 nm. All spectra were baseline-corrected. Molar mean residue ellipticity [θ] is expressed in degrees cm2 dmol-1, and calculated as [ν]θobs MWR/(101c) where θ is observed ellipticity in degrees; MWR, peptide mean residue molar weight; 1, the optical path length in cm, and c, peptide concentration in g/mL.

### Methods of Neuronal Cell Culture for Testing Effects of Aβ and LL-37 Peptides

Cells from the human neuroblastoma SH-SY5Y cell line were grown in DMEM/F12 medium containing 10 fetal bovine serum (FBS, Invitrogen, Carlsbad, Calif.) and 100 IU/mL penicillin and 100 g/mL streptomycin (Invitrogen, Carlsbad, Calif.) under humidified 5% CO₂ and 95% air.

Human microglial cells were isolated from surgically resected temporal lobe tissue as described in Lee et al. [REF: Depletion of GSH in glial cells induces neurotoxicity: relevance to aging and degenerative neurological diseases. Lee M, Cho T, Jantaratnotai N, Wang Y T, McGeer E, McGeer P L. FASEB J. 2010 July; 24(7):2533-45. Pubmed ID PMID 20228251]. Briefly, tissues were rinsed with a PBS solution and chopped into small pieces (<2 mm³) with a sterile scalpel. They were treated with 10 mL of a 0.25% trypsin solution at 37° C. for 20 min. Subsequently DNase I (from bovine pancreas, Pharmacia Biotech, Baie d'Urfé, PQ, Canada) was added to reach a final concentration of 50 μg/mL. Tissues were incubated for an additional 10 min at 37° C. After centrifugation a 275 g for 10 min, the cell pellet was resuspended in the serum-containing medium and passed through a 100 μm nylon cell strainer (Becton Dickinson. Franklin Lakes. N.J.). The cell suspension was centrifuged again (275 g for 10 min) and re-suspended in 10 mL of DMEM/F12 medium with 10% FBS containing gentamicin (50 μg/mL), and plated onto tissue culture plates (Becton Dickinson) in a humidified 5% CO₂,95% air atmosphere at 37° C. for 2 h. This achieved adherence of microglial cells. Cells were allowed to grow by replacing the medium once per week.

For estimating the purity of microglial cell cultures, aliquots of the cultures were placed on glass slides at 37° C. for 48 h. The attached cells were then fixed with 4% paraformaldehyde for 1 h at 4° C., and made permeable with 0.1% Triton X-100 for 1 h at room temperature. After washing twice with PBS, the culture slides were treated with the monoclonal anti-GFAP antibody ( 1/4,000, DAKO) for astrocytic staining and polyclonal anti-Iba-1 antibody ( 1/500, Wako Chemicals, Richmond, Va.) for microglial staining for 3 h at room temperature. The slides were then incubated with Alexa Fluor 488-conjugated goat anti-mouse IgG antibody (Invitrogen, 1:500) and Alexa Fluor 546-conjugated goat anti-rabbit IgG antibody (Invitrogen, 1:500) in the dark for 3 h at room temperature to yield a positive red fluorescence. To visualize all cells, the slides were washed twice with PBS and counterstained with the nuclear dye DAPI (100 μg/mL, Sigma) to give a blue fluorescent color. Images were acquired using an Olympus BX51 microscope and a digital camera (Olympus DP71). Fluorescent images were co-localized with ImagePro software (Improvision Inc., Waltham, Mass.). The purity of microglia was more than 99% (1.93±0.54 astrocytes in 500 total cells in microglial culture, n=30).

To achieve SH-SY5Y differentiation, the undifferentiated cells were treated for 4 days with 5 M retinoic aid (RA) in DMEM/F12 medium containing 5% FBS, 100 IU/mL penicillin, and 100 g/mL streptomycin [REF: issue transglutaminase mediates activation of RhoA and MAP kinase pathways during retinoic acid-induced neuronal differentiation of SH-SY5Y cells. Singh U S, Pan J, Kao Y L, Joshi S, Young K L, Baker K M. J Biol Chem. 2003 Jan. 3; 278(1):391-9. Pubmed ID PMID 12401808]. The RA-containing medium was changed every two days. Differentiated SH-SY5Y cells demonstrated neurite extension, indicative of their differentiation [REF: Neurotoxins released from interferon-gamma-stimulated human astrocytes. Lee M, McGeer E, McGeer P L. Neuroscience. 2013 Jan. 15; 229:164-75. Pubmed ID PMID 23098801].

Methods for Neuronal Cell Culture Treatment with Aβ and LL-37 Peptides

Human microglial cells (5×10⁴ cells per well) or human neuroblastoma SH-SY5Y cells (5×10⁵ cells per well) or their mixture were seeded into 24-well plates in 1 mL of DMEM/F12 medium containing 5% FBS. Aβ42 peptide (AnaSpec, Fremont, Calif.) was dissolved in mixture of 1% NH4OH and 0.1% Thioflavin S (Sigma, St. Louis, Mo.) in water to make 30 mM stock solution. Further dilution was made with water to reach 30 μM. Similar methods were used to prepare LL-37 peptide solutions (AnaSpec, Fremont, Calif.). Cultured cells were exposed to either 30 μM Aβ42 or 30 μM LL-37; or to a combined 1:1 equimolar mixture of the two peptides at 30 μM each. After incubation for 72 h cell free medium was collected to measure levels of proinflammatory cytokines such as TNFα and IL-6. For SH-SY5Y cell viability, MTT assays were performed as described by Lee et al. [REF: Acidic fibroblast growth factor (FGF) potentiates glial-mediated neurotoxicity by activating FGFR2 IIIb protein. Lee M, Kang Y, Suk K, Schwab C, Yu S, McGeer P L. J Biol Chem. 2011 Dec. 2; 286(48):41230-45. Pubmed ID PMID 21990352].

### Methods of SH-SY5Y-Microglial Cell Viability Assays

The viability of SH-SY5Y cells following incubation with glial cell supernatants was evaluated by MTT assays as described by Lee et al. [REF: Acidic fibroblast growth factor (FGF) potentiates glial-mediated neurotoxicity by activating FGFR2 IIIb protein. Lee M, Kang Y, Suk K, Schwab C, Yu S, McGeer P L. J Biol Chem. 2011 Dec. 2; 286(48):41230-45. Pubmed ID PMID 21990352]. Briefly, the viability was determined by adding MTT to the cell cultures to reach a final concentration of 1 mg/mL. Following 1 h incubation at 37° C., the dark crystals formed were dissolved by adding a SDS/DMF extraction buffer (300 μL, 20% sodium dodecyl sulfate, 50% N, N-dimethylformamide, pH 4.7). Subsequently plates were incubated overnight at 37° C. and optical densities at 570 nm were measured by transferring 100 μL aliquots to 96-well plates and using a plate reader with a corresponding filter. Data are presented as a percentage of the values obtained from cells incubated in fresh medium only.

Methods for the Measurement of TNF and IL-6 Release from Cultured Neuronal Cells

Cytokine levels were measured in cell-free supernatants after 72 h. Quantitation was performed with ELISA detection kits (Peprotech, NJ) following protocols described by the manufacturer.

### Methods for Statistical Data Analysis of Results Obtained in Neuronal Cell Culture Assays

The significance of differences between data sets was analyzed by one-way ANOVA tests. Multiple group comparisons were followed by a post-hoc Bonferroni test, e.g. cell viability in control condition versus cells treated with LL-37 alone; cell viability in control condition versus cells treated with Aβ42 alone; and viability in control condition versus cells treated with mixed LL-37+Aβ42. P values are provided in the legends of the figures that show the results.

### Example 1—SPRi Analysis of LL-37 Binding to Immobilized Aβ; and CE Analysis

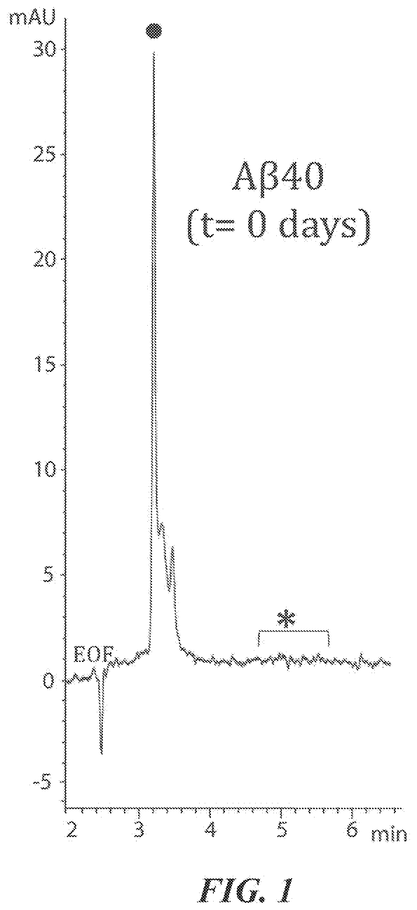

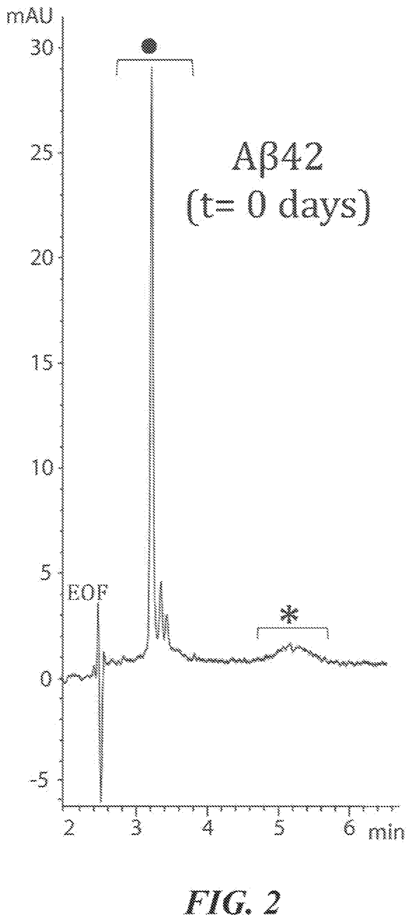

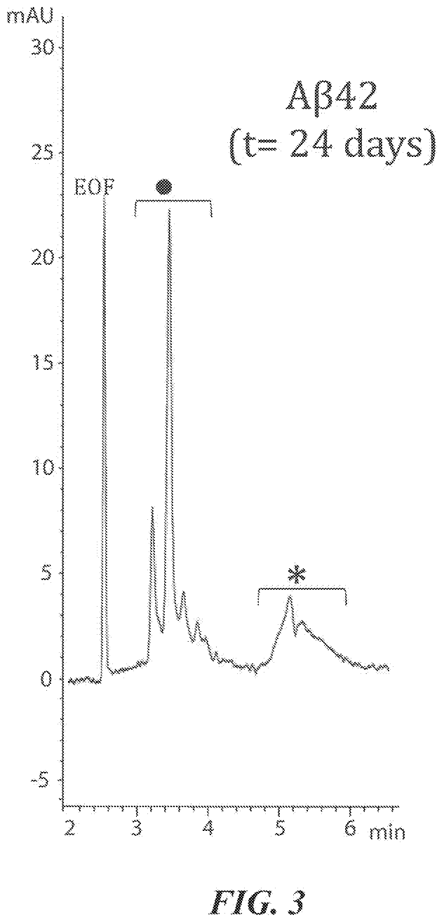

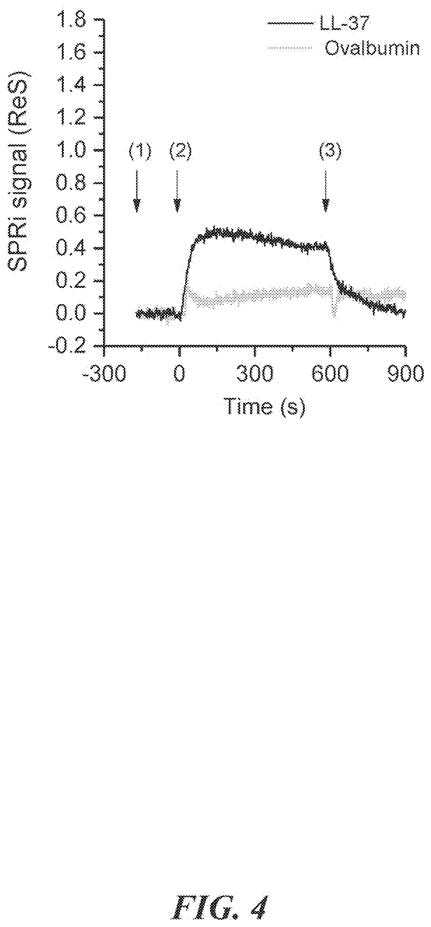

To demonstrate interaction between LL-37 and Aβ42, a Surface Plasmon Resonance imaging (SPRi) biochip was functionalized with copoly(DMA-NAS-MAPS), a polydimethylacrylamide based copolymer, widely used to immobilize biomolecules on microarray slides [REF: Overcoming mass transport limitations to achieve femtomolar detection limits on silicon protein microarrays. Cretich M, Bagnati M, Damin F, Sola L, Chiari M. Anal Biochem. 2011 Nov. 1, 418(1):164-6. Pubmed ID PMID 21802399]. Three different solutions of 20 μM Aβ-peptides, in different states of aggregation, were spotted onto the SPRi-chip surface. The aggregation state and the presence of soluble oligomers were determined by CE analysis [REF: Disease-modifying anti-Alzheimer's drugs: inhibitors of human cholinesterases interfering with β-amyloid aggregation. Brogi S, Butini S, Maramai S, Colombo R, Verga L, Lanni C, De Lorenzi E, Lamponi S, Andreassi M, Bartolini M, Andrisano V, Novellino E, Campiani G, Brindisi M, Gemma S. CNS Neurosci Ther. 2014 July; 20(7):624-32. Pubmed ID PMID 24935788] and [REF: Capillary electrophoresis studies on the aggregation process of beta-amyloid 1-42 and 1-40 peptides. Sabella S, Quaglia M, Lanni C, Racchi M, Gavoni S, Caccialanza G, Calligaro A, Bellotti V, De Lorenzi E. Electrophoresis. 2004 October; 25(18-19):3186-94. Pubmed ID PMID 15472964]. Aβ40 (a) dissolved in PBS, Aβ42 (b) prepared as in [16], both solubilized immediately before spotting (t=0 days), and Aβ42 (c) prepared as sample (b), but stored in the buffer for 24 days (t=24 days) were spotted in different subarrays. Aβ42 was dissolved following the protocol detailed in the Material and Methods section. The solubilization buffer consisted of i) HFIP, a low-polarity solvent that stabilizes the α-helix and disrupts the interstrand hydrogen bonds of the β-sheets and ii) a mixture of acetonitrile/Na2CO3/NaOH. While acetonitrile stabilizes the unordered/α-helix structure, the basic pH increases the solubility of Aβ peptide. Overall, this procedure disfavors aggregation and provides a time window long enough to appreciate the prevailing formation of low-MW oligomers at early stages (t=0 days) and the building up of high-MW oligomers at late-stages (t=24 days) of the in vitro fibrillogenesis process. Indeed, this was confirmed by analyzing in CE the three samples at 100 μM concentration using a method slightly modified from Sabella et al. [REF: Disease-modifying anti-Alzheimer's drugs: inhibitors of human cholinesterases interfering with β-amyloid aggregation. Brogi S, Butini S, Maramai S, Colombo R, Verga L, Lanni C, De Lorenzi E, Lamponi S, Andreassi M, Bartolini M, Andrisano V, Novellino E, Campiani G, Brindisi M, Gemma S. CNS Neurosci Ther. 2014 July; 20(7).624-32. Pubmed ID PMID 249357988. FIGS. 1-3 show CE electropherograms of the same Aβ peptide solutions spotted on the SPRi chip; consistent with what has been reported previously in literature [REF: Disease-modifying anti-Alzheimer's drugs-inhibitors of human cholinesterases interfering with β-amyloid aggregation. Brogi S, Butini S, Maramai S, Colombo R, Verga L, Lanni C, De Lorenzi E, Lamponi S, Andreassi M, Bartolini M, Andrisano V, Novellino E, Campiani G, Brindisi M, Gemma S. CNS Neurosci Ther. 2014 July; 20(7):624-32. Pubmed ID PMID 24935788] and [REF: Capillary electrophoresis studies on the aggregation process of beta-amyloid 1-42 and 1-40 peptides. Sabella S, Quaglia M, Lanni C, Racchi M, Gavoni S, Caccialanza G, Calligaro A, Bellotti V, De Lorenzi E. Electrophoresis. 2004 October; 25(18-19):3186-4. Pubmed ID PMID 15472964] and [REF: CE can identify small molecules that selectively target soluble oligomers of amyloid beta protein and display antifibrillogenic activity. Colombo R I, Carotti A, Catto M, Racchi M, Lanni C, Verga L, Caccialanza G, De Lorenzi E. Electrophoresis. 2009 April; 30(8):1418-29. Pubmed ID PMID 193062691, the fast-migrating group of peaks can be attributed to low MW-oligomers whereas the slow-migrating broad band corresponds to high MW-oligomers. In particular, in the Aβ42 sample, the high MW-oligomers build up over time, as evident in FIG. 2 and FIG. 3.

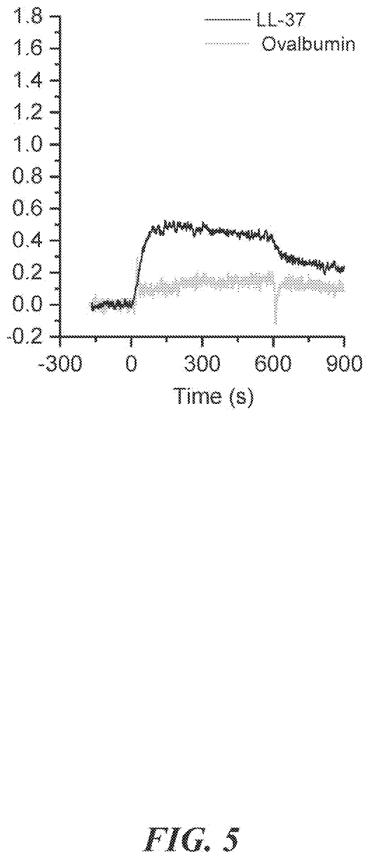

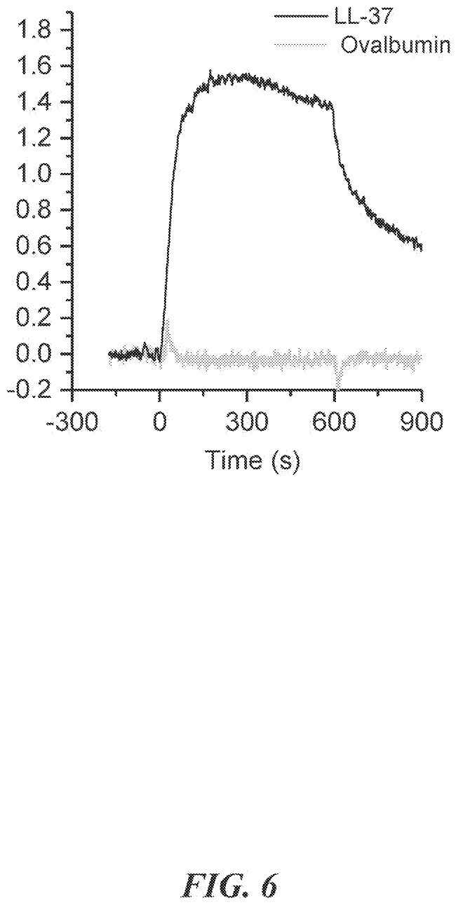

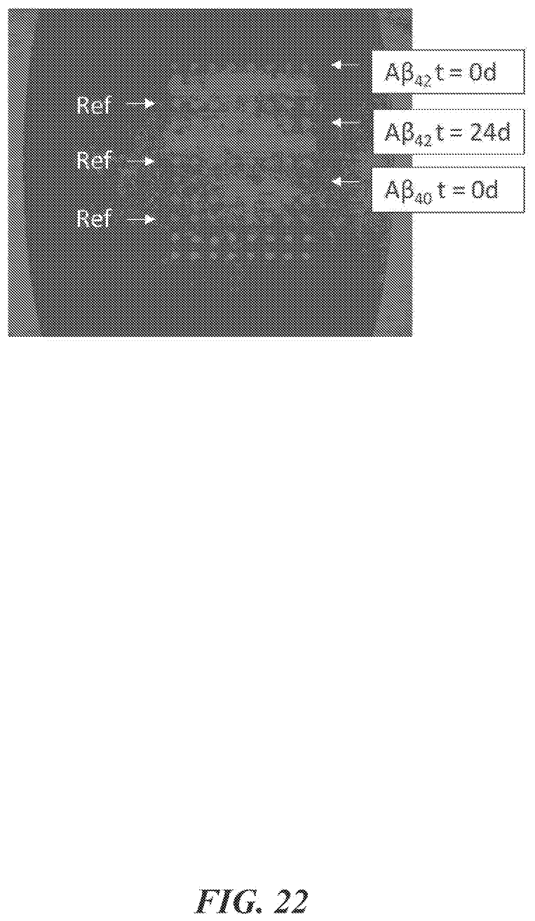

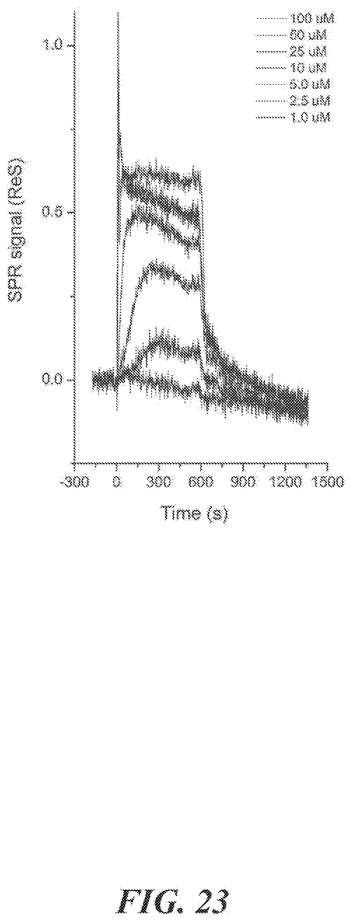

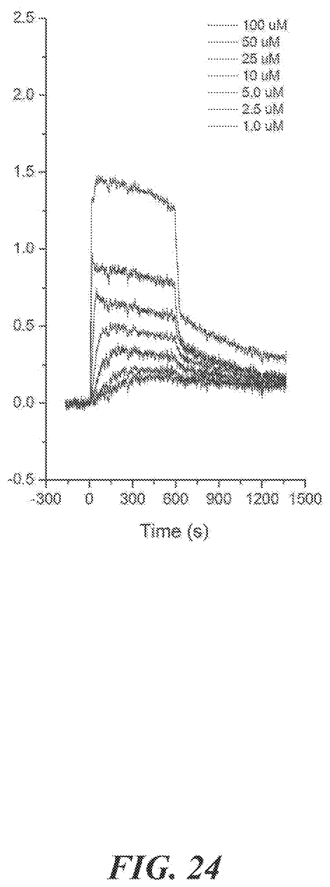

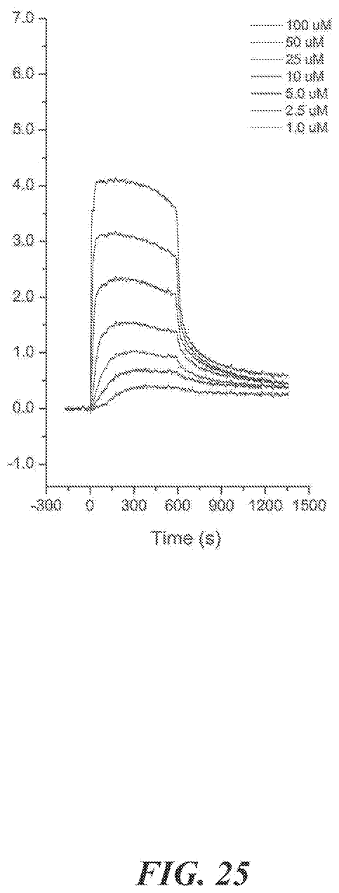

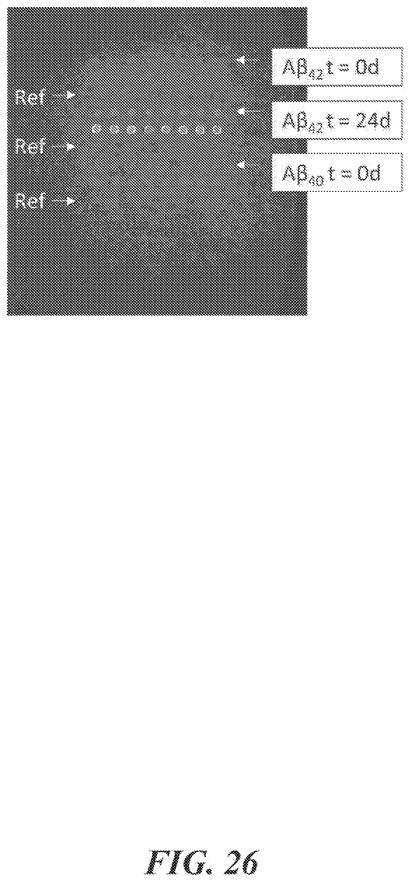

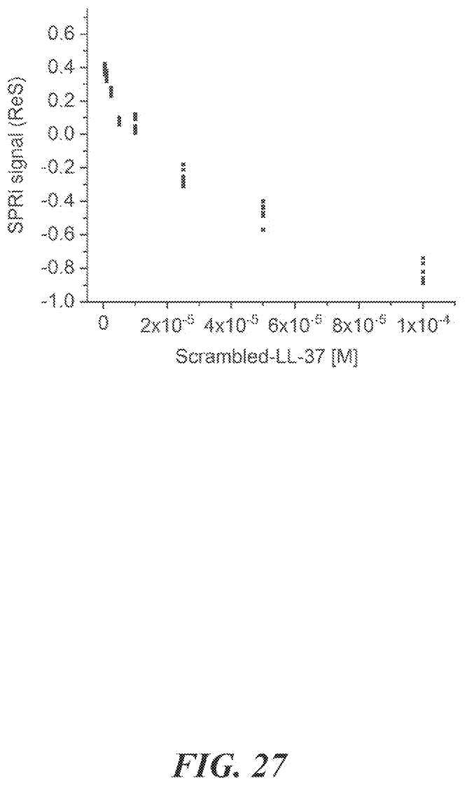

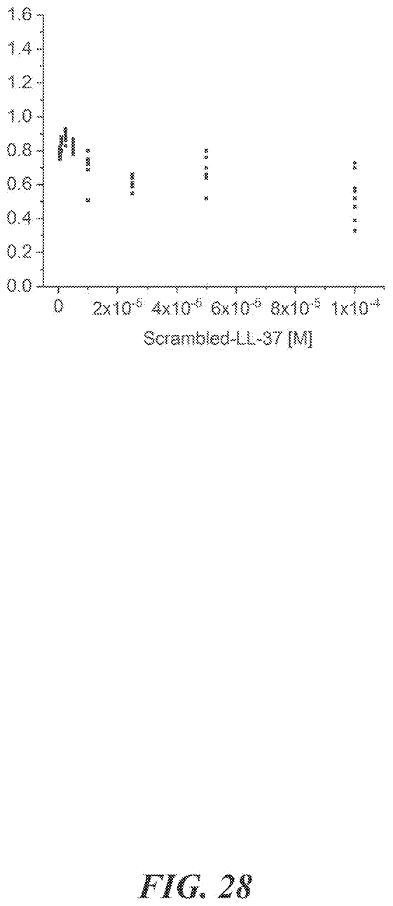

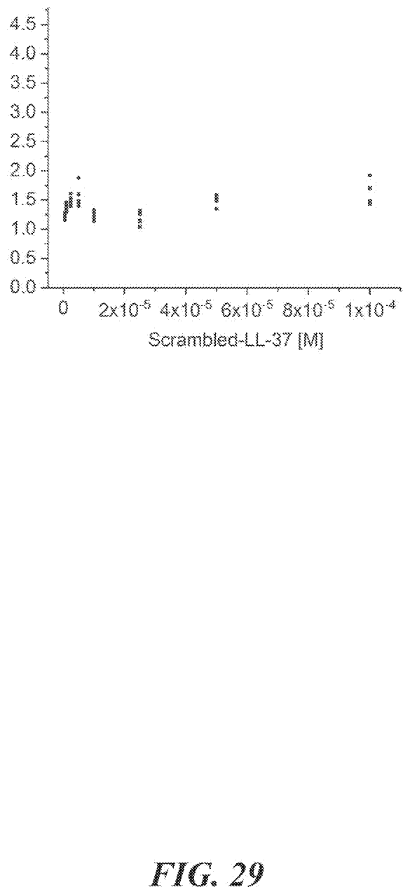

The interaction of Aβ in different aggregation states with LL-37 was investigated by SPRi. The CCD image of the chip surface, and the plasmonic curves acquired before the injection of LL-37 (FIG. 22), show the three Aβ forms conjugated to the surface. Specifically, the plasmonic curves of the different samples indicate that the sample containing a higher amount of Aβ42 high-MW oligomers (t=24 days) binds to the surface more, compared to Aβ42 low-MW oligomers (t=0 days) which, in turn, binds mom than Aβ40 low-MW oligomers (t=0 days). Differences in the immobilized mass could result from the increased molecular weight of the samples in different aggregation states from low to high MW-oligomers [REF: Kinetic characterization of amyloid-beta 1-42 aggregation with a multimethodological approach. Bartolini M, Naldi M, Fiori J, Valle F, Biscarini F, Nicolau D V, Andrisano V. Anal Biochem 2011 Jul. 15; 414 (2).215-25. Pubmed ID PMID 21435333] and [REF: Disease-modifying anti-Alzheimer's drugs: inhibitors of human cholinesterases interfering with β-amyloid aggregation. Brogi S, Butini S, Maramai S, Colombo R, Verga L, Lanni C, De Lorenzi E, Lamponi S, Andreassi M, Bartolini M, Andrisano V, Novellino E, Campiani G, Brindisi M, Gemma S. CNS Neurosci Ther 2014 July, 20(7):624-32. Pubmed ID PMID 24935798]. The sensograms taken after flowing a solution of LL-37 onto the SPRi chip for 10 minutes, revealed specific and reversible interactions with all the three Aβ forms (FIG. 2-4). The injection of ovalbumin, a non-correlated protein, did not produce interaction signals Analogously, the amount of LL-37 captured by non-correlated peptides (negative controls), spotted in parallel on the same SPRi chip, was significantly lower compared to that of positive signals. In a follow-up experiment, seven serial dilutions of LL-37, ranging from 1 to 100 μM, were injected to investigate the dependency of the SPR signal on ligand concentration for affinity studies (FIGS. 23-25). An equilibrium analysis (steady-state analysis) was performed by fitting the SPRi response at the equilibrium state for each concentration (FIGS. 7-9). It is noteworthy that the system reached the equilibrium state at each LL-37 concentration and a complete regeneration of the SPRi chip surface was obtained after each injection. As expected, the maximal response value, or maximum binding capacity (Rₘₐₓ), which is related to the maximum number of LL-37 peptides that can bind the Aβ peptides at a certain time, decreases in the following order: Aβ42 (t=24 days)>>Aβ42 (t=0 days)>Aβ40 (t=0 days) (FIGS. 4-9, TABLE 1 below). This is in agreement with the amount of Aβ peptide immobilized on the SPRi chip, which increased with Aβ aggregation. To the contrary, as shown in TABLE 1 below, the affinity of LL-37 for the different AP forms shows the opposite trend, i.e. LL-37 binds to low-MW Aβ oligomers more strongly than to high-MW Aβ oligomers. The binding specificity of LL-37 was verified by performing the same equilibrium analysis with a control scrambled-sequence LL-37. For this experiment, it was difficult to extract adequate calibration curves (see FIGS. 27-29), mostly due to non-specific and irregular interactions. For this reason, the binding affinities constants could not be calculated for experiments using scrambled LL-37.

TABLE 1
Values of the maximal response (Rₘₐₓ), (related to the absolute
number of LL-37 molecules bound on the Aβ peptides at a certain
time) and equilibrium binding constants (K_{A} and K_{D}), calculated
using a nonlinear curve fit of the SPRi response at equilibrium.

| Peptide | Rₘₐₓ | K_{A} (M⁻¹) | K_{D} (μM) |
|---|---|---|---|
| Aβ₄₀ (t = 0 days) | 0.59 | 1.20 × 10⁵ | 8.3 |
| Aβ₄₂ (t = 0 days) | 1.56 | 7.51 × 10⁴ | 13.3 |
| Aβ₄₂ (t = 24 days) | 4.43 | 4.92 × 10⁴ | 20.3 |

### Example 2—Results of Transmission Electron Microscopy Analysis of Aβ and LL-37 Peptides

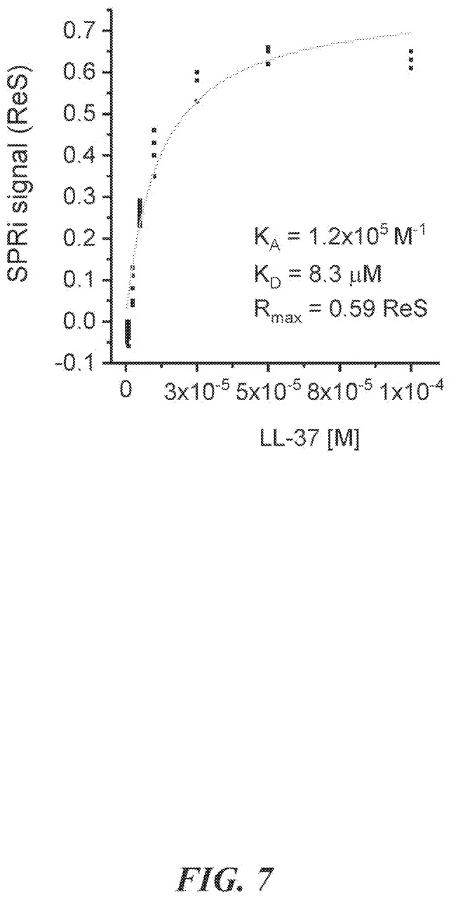

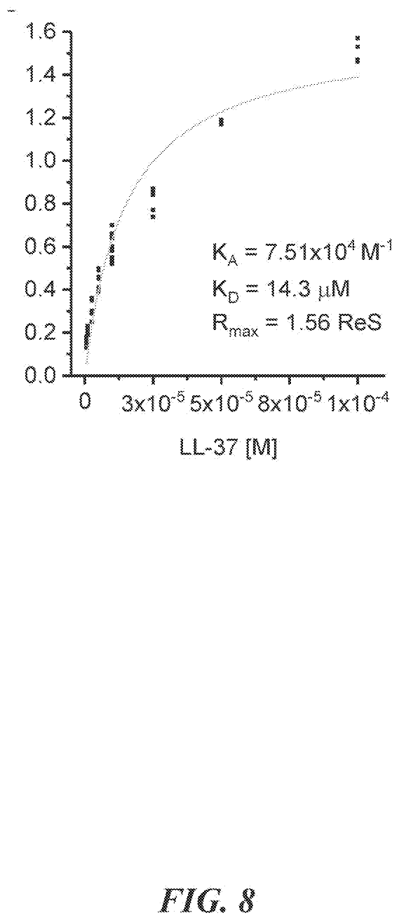

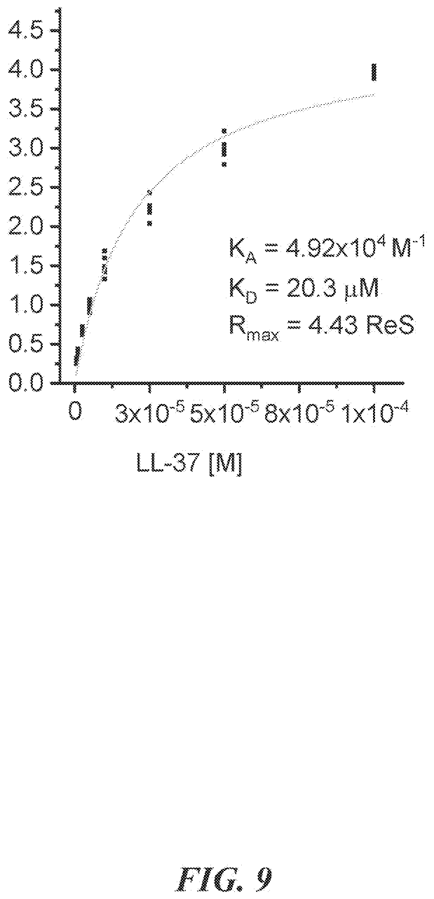

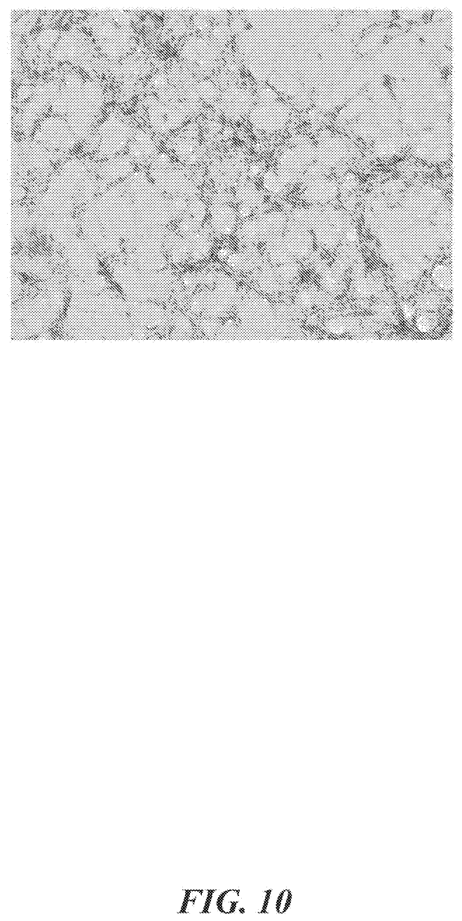

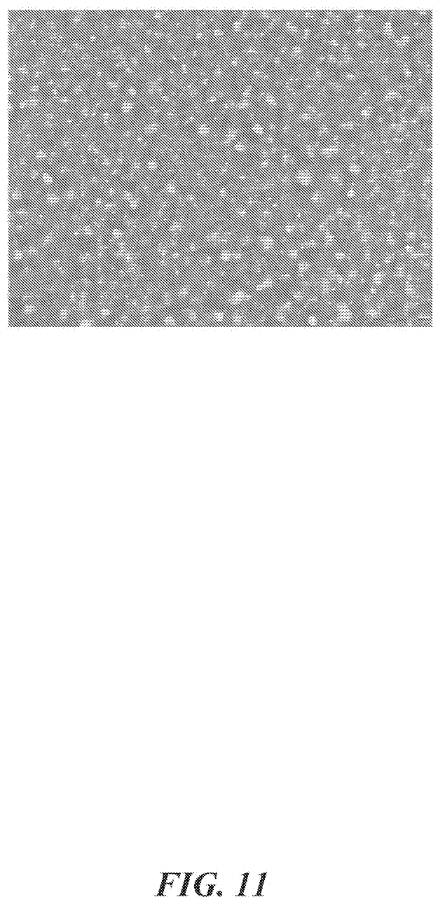

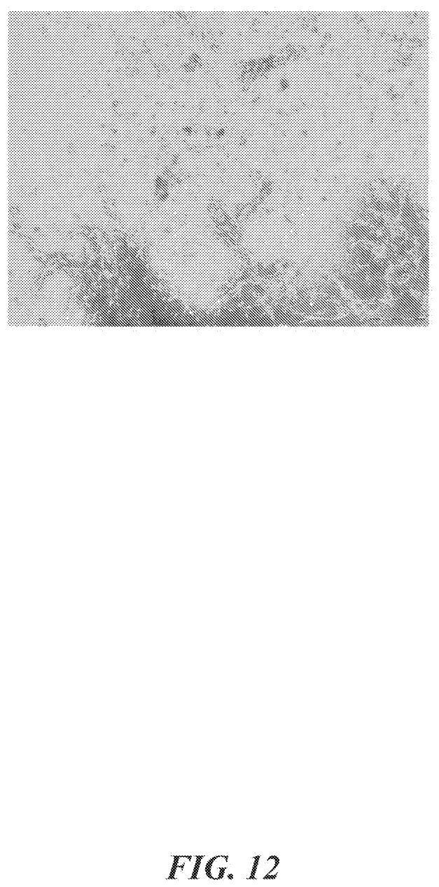

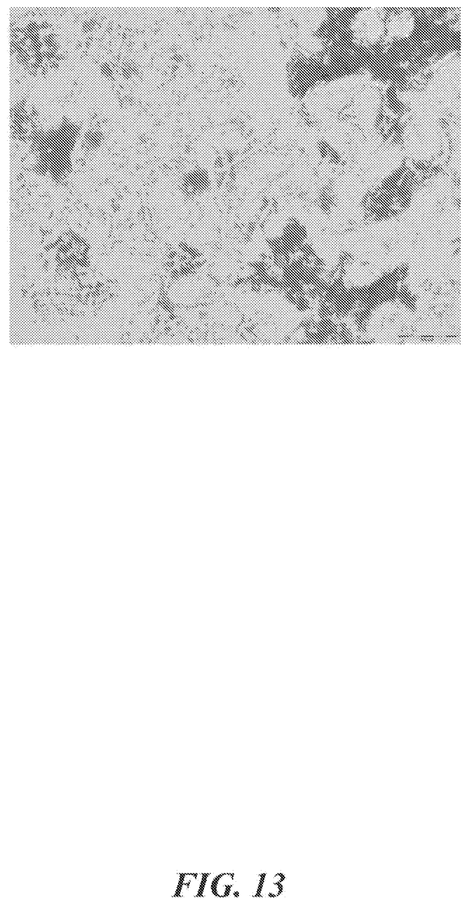

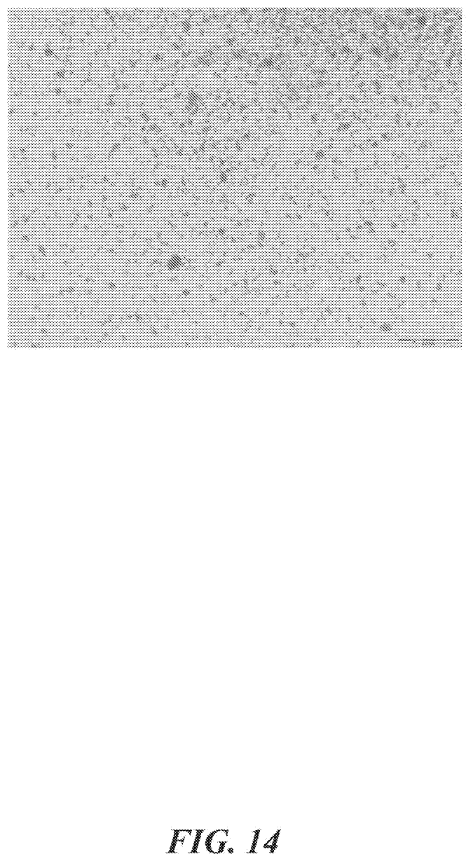

An inhibitory effect of LL-37 peptide binding on fibril formation was demonstrated by transmission electron microscopy (TEM). For these experiments (see Methods), samples were prepared according to a more aggregating protocol [REF: Disease-modifying anti-Alzheimer's drugs: inhibitors of human cholinesterases interfering with β-amyloid aggregation. Brogi S, Butini S, Maramai S, Colombo R, Verga L, Lanni C, De Lorenzi E, Lamponi S, Andreassi M, Bartolini M, Andrisano V, Novellino H, Campiani G, Brindisi M, Gemma S. CNS Neurosci Ther. 2014 July; 20(7):624-32. Pubmed ID PMID 24935788], to mimic quasi-physiological conditions and to better verify LL-37 anti-fibrillogenic activity. Aβ42 reproducibly forms a dense network of interpenetrating, μm-long, straight, unbranched filaments with a diameter of about 10 nm (FIG. 10, n=3), corresponding to known features of classic, mature amyloid fibrils [REF: Molecular mechanisms of amyloidosis. Merlini G, Bellotti V. N Engl J Med. 2003 Aug. 7; 349(6):593-96. Pubmed ID PMID: 12904524]. This network of long, straight, interconnected fibrils covered the TEM plate uniformly. On the other hand, LL-37 alone in solution produced globular, amorphous aggregates (FIG. 11, n=3). To assess the influence of LL-37 on A fibril formation, 50 μM Aβ42 was co-incubated with an equimolar amount of LL-37 peptide. Mixtures of Aβ42 and LL-37, prepared as described in Methods were analyzed by TEM, either immediately after solubilization or after 3 or 9 days of incubation. In the equimolar solution of the two peptides at t=0, shortened, less defined, more sparsely represented fibrils (as compared to FIG. 10) were observed, in mixture with clumps of amorphous material (FIG. 12, n=2). These evolved into (as compared to FIG. 10) sparser dumps of shorter and more curved fibrils at t=3 days (FIG. 13, n=2), and then into globular amorphous aggregates at t=9 days, with no visible fibrils present (FIG. 14, n=2). As reported in literature, molecules with a similar time-dependent antifibrillogenic effect are considered inhibitors of Aβ42 fibril formation [REF: N-Methylated peptide inhibitors of beta-amyloid aggregation and toxicity. Optimization of the inhibitor structure. Kokkoni N, Stott K, Amijee H, Mason J M, Doig A J. Biochemistry. 2006 Aug. 15; 45(32):9906-18. Pubmed ID PMID 16893191] and [REF: Discovery and structure activity relationship of small molecule inhibitors of toxic β-amyloid-42 fibril formation. Kroth H, Ansaloni A, Varisco Y, Jan A, Sreenivasachary N, Rezaei-Ghaleh N, Giriens V, Lohmann S, López-Deber M P, Adolfsson O, Pihlgren M, Paganetti P, Froestl W, Nagel-Steger I, Willbold D, Schrader T, Zweckstetter M, Pfeifer A, Lashuel H A. Muhs A. J Biol Chem. 2012 Oct. 5; 287(41):34786-800. Pubmed ID PMID 22891248], thus from these data it can be deduced that LL-37 inhibits Aβ42 fibril formation.

### Example 3—Results of Conformational Analysis by Circular Dichroism (CD) Spectroscopy

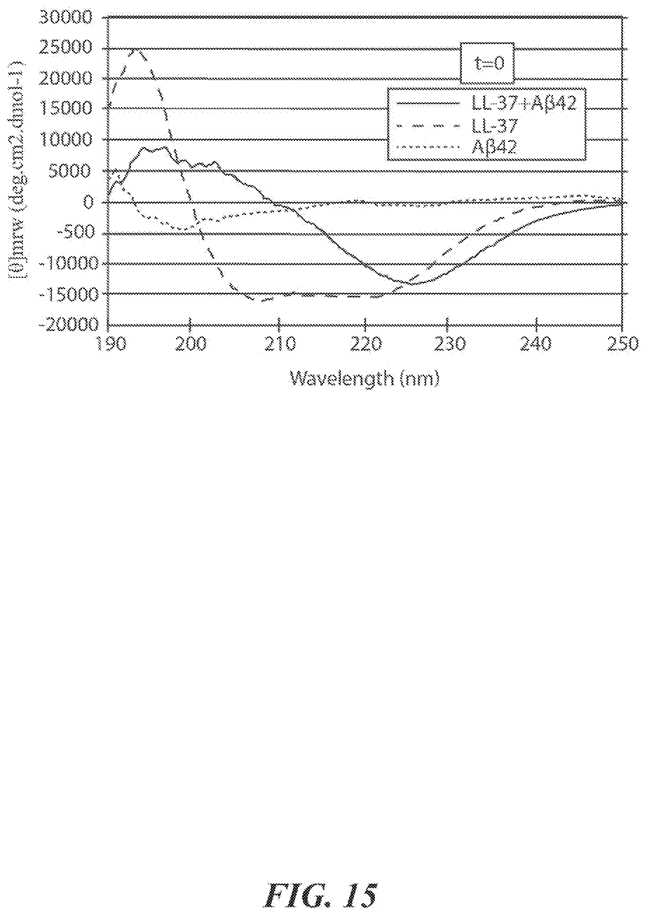

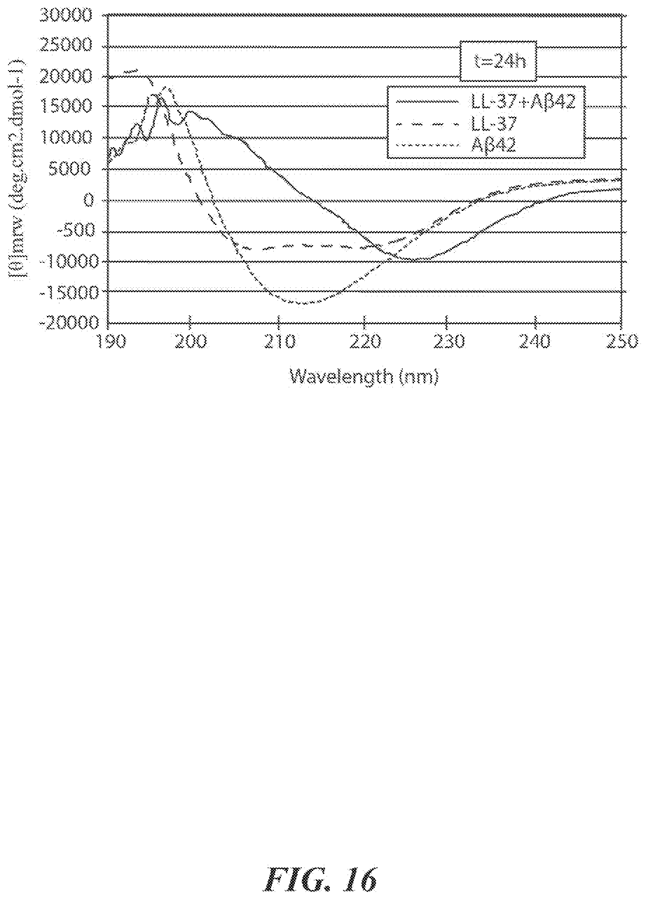

Conformational analyses of Aβ42 peptide in solution, in the absence and presence of LL-37, were carried out by circular dichroism (CD) spectroscopy. As in TEM analysis, spectra were recorded immediately after dissolving peptide according to [REF: Disease-modifying anti-Alzheimer's drugs: inhibitors of human cholinesterases interfering with β-amyloid aggregation. Brogi S, Butini S, Maramai S, Colombo R, Verga L, Lanni C, De Lorenzi E, Lamponi S, Andreassi M, Bartolini M, Andrisano V, Novellino E, Campiani G, Brindisi M, Gemma S. CNS Neurosci Ther. 2014 July; 20(7).624-32. Pubmed ID PMID 24935788] (FIG. 15) and again after 24 hours (FIG. 16). We found that at t=0, Aβ42 assumed an unordered conformation (FIG. 15) whereas over 24 hours, adoption of β-type conformations was observed (FIG. 16), with a characteristic positive band at 195 nm and negative band at 215 nm. On the other hand, 50 μM LL-37 at t=0, immediately adopted α-helical conformations and maintained this secondary structure over 24 hours (FIG. 15-16). Remarkably, in the presence of LL-37, the Aβ42 peptide in solution seemed to be prevented, to a large degree, from forming typical β structure (FIGS. 15-16).

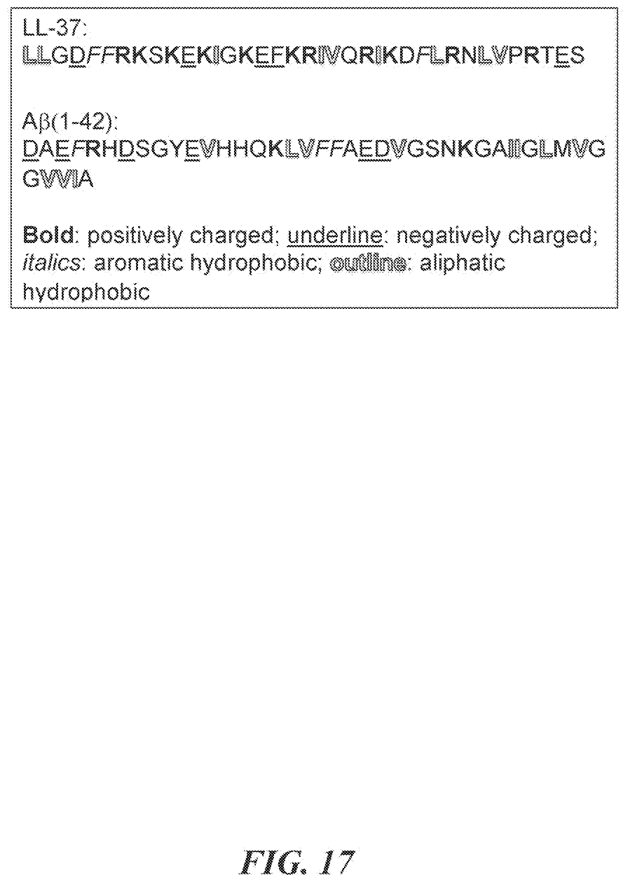

While CD is essentially an “averaging” spectroscopic method, the spectra of 1:1 mixed Aβ42 and LL-37 peptides do not show ordered β structure of the type that predominates in pure Aβ42 solutions. No time-dependence, within 24 hours, of peptide secondary structure was observed in the mixed solutions, in contrast to the behavior of Aβ42 solutions. As discussed below, this interaction between the peptides makes sense, considering their opposite charges and mutually high and similar proportions of aromatic and aliphatic amino acids (FIG. 17). We can only conclude, definitively, that, in the presence of LL-37 we do not observe the typical CD spectrum of self-associated Aβ42.

### Example 4—Effects of Aβ42, LL-37, and Aβ42/LL-37 Complexes on Human Microglia, SH-SY5Y Neuronal Cells, and Microglia-Mediated Cytotoxicity to SH-SY5Y Neuronal Cells

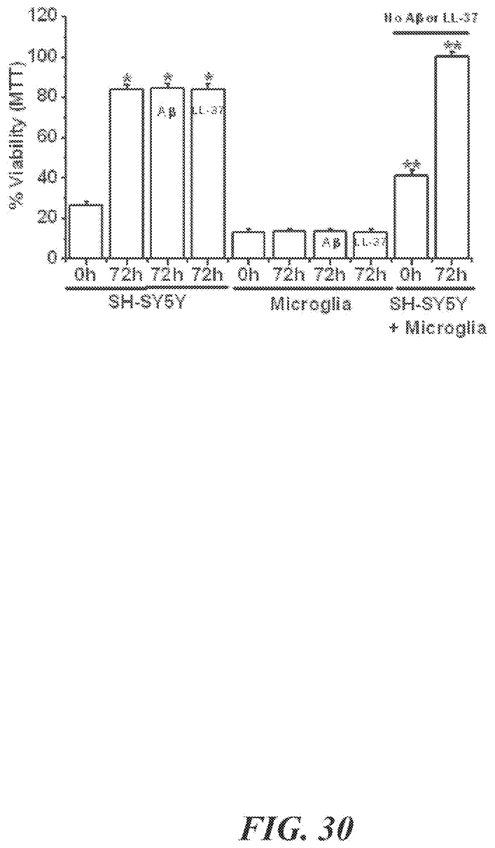

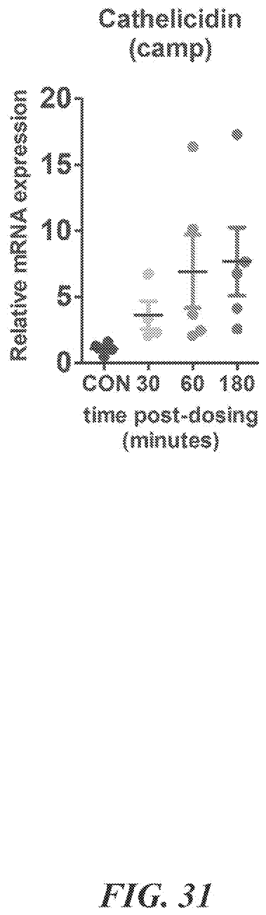

We studied the effects of Aβ42 alone, LL-37 alone, and Aβ42/LL-37 complexes on SH-SY5Y neuroblastoma cells, human microglia, and on cytokine release from microglia that impact SH-SY5Y cells. Specifically, we exploited a simplified model that reproduces the cross-talk between the major players involved in neuronal damage by looking at the effect of cell-free supernatant from microglial cell culture, on cultured neuronal cells. Cultured cells were exposed to either Aβ42 alone or LL-37 alone (each at 30 μM concentration). After incubation for 72 h, MTT assays were performed and the cell-free medium was collected to measure levels of proinflammatory cytokines TNFα and IL-6. Note, as shown in the control experiments (FIG. 30) neither Aβ nor LL-37 peptide was toxic to SH-SY5Y cells directly, nor to human microglia alone, at any concentration.

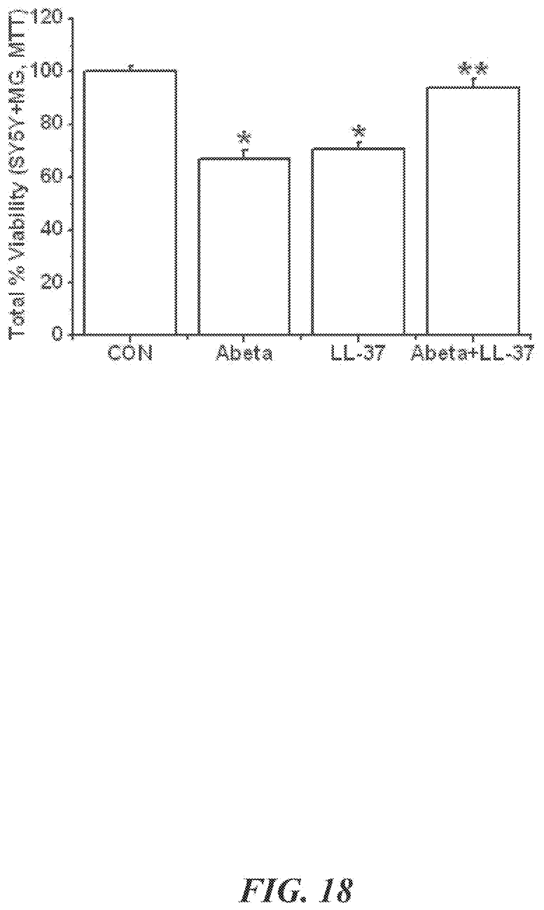

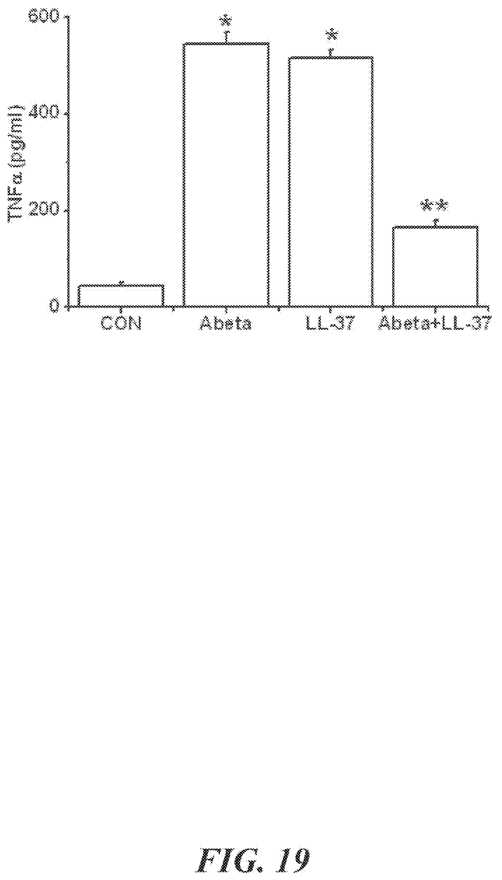

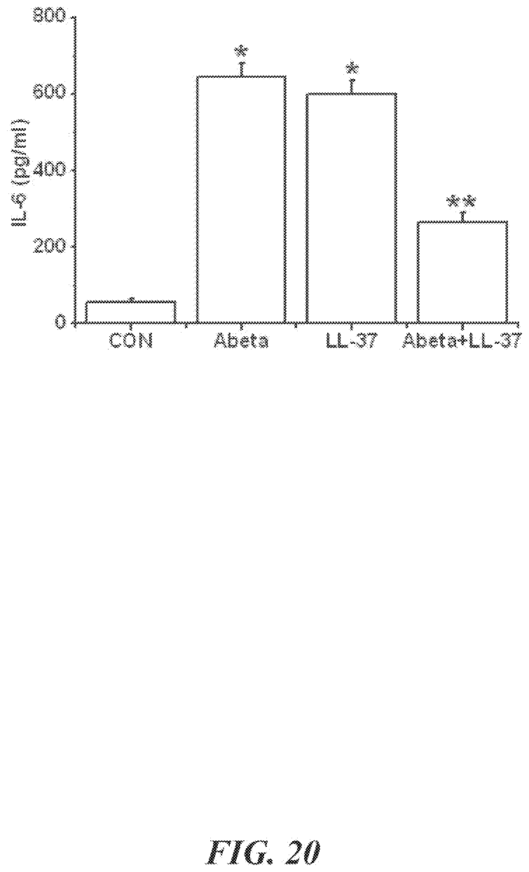

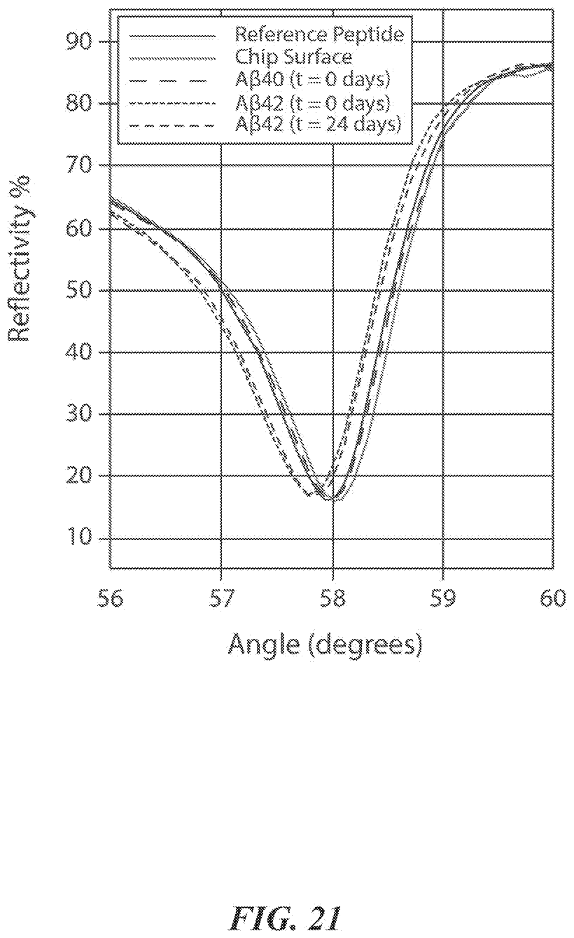

However, we find evidence for microglia-mediated neuroinflammation, for both peptides. Human microglia were exposed to either Aβ alone, LL-37 alone, or an equimolar LL-37/Aβ mixture for 2 days. Then cell-free supernatants from microglia cultures were transferred to differentiated SH-SY5Y cells. SH-SY5Y cell viability was measured after 72 h. We also measured TNFα and IL-6 levels in microglial cell-free supernatants after 2 days' incubation with the peptides. Data are presented in FIGS. 18-20. Treatment of microglia with Aβ or LL-37 induced release of toxic microglial cytokines TNFα and IL-6 (FIG. 19 and FIG. 20) and resulted in a reduction in SH-SY5Y cell viability (down to 70% after 3 days, FIG. 13). However, when microglia were exposed to an equimolar Aβ:LL-37 mixture (30 μM each), the complexation of these two peptides reduced TNFα and IL-6 release, and attenuated SH-SY5Y cell viability loss by more than 90% which is a major reduction in the deleterious impacts of either peptide, i.e., their complex is non-toxic to neuronal cells.

### Discussion of In Vitro Results of Aβ:LL-37 Interaction Studies by TEM, CD, SPRi, and Neuronal Cell Culture

Among the many inhibitors of Aβ aggregation and neurotoxicity that have been identified previously by various assays, or designed, synthesized, and/or selected. [REF: Molecules that target beta-amyloid. Stains C I, Mondal K, Ghosh I, ChemMedChem 2007 December; 2(12):1674-92. Review. Pubmed ID PMID: 17952881] and [REF: A molecular chaperone breaks the catalytic cycle that generates toxic Aβ oligomer. Cohen S I A, Arosio P, Presto J, Kurudenkandy F R, Biverstal H, Dolfe L, Dunning C, Yang X, Frohm B, Vendruscolo M, Johansson J, Dobson C M, Fiaahn A, Knowles T P J, Linse S. Nat Struct Mol Biol 2015 March; 22(3):207-213. Pubmed ID PMID 25686037] However, no naturally occurring peptides that match Aβ in size were ever identified. The cathelicidin LL-37 is a pleiotropic, 37-residue innate immune effector that corresponds to residues 134-170 of the human cationic antimicrobial protein [REF: The chemistry and biology of LL-37. Burton M F, Steel P G. Nat Prod Rep 2009 December; 26(12):1572-84. Review. PMID 19936387]. It is one of a kind in the human proteome and is expressed in many organs, including the brain [REF: The chemistry and biology of LL-37. Burton M F, Steel P G. Nat Prod Rep. 2009 December; 26(12):1572-84. Review. PMID: 19936387] and [REF: Human antimicrobial peptide LL-37 induces glial-mediated neuroinflammation. Lee M, Shi X I, Barron A P, McGeer E, McGeer P L. Biochem Pharmacol. 2015 Mar. 15; 94(2):130-41. Pubmed ID PMID 25686659]. LL-37 fulfills myriad systemic roles as an immune system effector and modulator [REF: Mol Biosyst. 2009 May; 5(5):483-96. Systems biology evaluation of immune responses induced by human host defence peptide LL-37 in mononuclear cells. Mookherjee N I, Hamill P, Gardy J, Blimkie D, Falsafi R, Chikatamarla A, Arenillas D J, Doria S, Kollmann T R, Hancock RE. Pubmed PMID 1931363] Additionally, LL-37's immunomodulatory functions and effects on cells, links the endocrine and immune systems. [REF: Cathelicidins link the endocrine and immune systems. Mayer M L, Hancock R E Cell Host Microbe. 2010 Apr. 22; 7(4):257-9. Pubmed ID PMID 20413092] Moreover, LL-37 plays a central role in Vitamin D3-stimulated monocyte/macrophage autophagy. [REF: Vitamin D3 induces autophagy in human monocytes/macrophages via cathelicidin. Yuk J M, Shin D M, Lee H M, Yang C S, Jin H S, Kim K K, Lee Z W, Lee S H, Kim J M, Jo E K. Cell Host Microbe 2009 Sep. 17; 6(3):231-43. Pubmed ID PMID 19748465]

Thus, these experiments that were described above, have both discovered and quantified a sequence-specific molecular binding interaction between Aβ and LL-37, indicating that these two peptides appear to be natural binding partners. If so, the dynamics of spatiotemporal expression of LL-37 and Aβ are likely to hold major significance to human health. While there are myriad well-studied functions of LL-37 as an antimicrobial and antiviral weapon of immune cells, the physiological functions of Aβ are not yet fully elucidated [REF: Physiological roles for amyloid beta peptides. Pearson H A, Peers C. J Physiol. 2006 Aug. 15; 575 (Pt 1):5-10. Pubmed PMID: 16809372]. Recently published work suggests that Aβ may itself function in vivo, particularly in the human brain, as an antimicrobial and antiviral peptide [REF: The Alzheimer's disease-associated amyloid beta-protein is an antimicrobial peptide. Soscia S J, Kirby J E, Washicosky K J, Tucker S M, Ingelsson M, Hyman B, Burton M A, Goldstein L E, Duong S, Tanzi R E, Moir R D. PLoS One. 2010 Mar. 3; 5(3):e9505. Pubmed ID PMID: 20209079] and [REF: Sci Transl Med. 2016 May 25; 8(340):340ra72. Amyloid-β peptide protects against microbial infection in mouse and worm models of Alzheimer's disease. Kumar D K, Choi S H, Washicosky K J, Eimer W A, Tucker S, Ghofrani J, Lefkowitz A, McColl G, Goldstein L E, Tanzi R E, Moir R D. Pubmed ID PMID 27225182] and [REF: Biogerontology. 2015 February; 16(1):85-98. β-Amyloid peptides display protective activity against the human Alzheimer's disease-associated herpes simplex virus-1. Bourgade K, Garneau H, Giroux G, Le Page A Y, Bocti C, Dupuis G, Frost E H, Fülöp T Jr. Pubmed ID PMID 25376108] and [REF: Alzheimer's Disease-Associated β-Amyloid Is Rapidly Seeded by Herpesviridae to Protect against Brain infection. Eimer W A, Kumar D K V, Shanmugam N K N, Rodriguez A S, Mitchell T, Washicosky K J, György B, Breakefield X O, Tanzí RE, Moír R D. Neuron. 2018 Jul. 11; 99(1):56-63].

The study of LL-37:Aβ interaction was initially inspired by an observation of a strong, apparent complementarity between the peptides' amino acid (aa) sequences (FIG. 17). They are similar in size and molecular weight (Aβ: 39-42 aa, Aβ42 is 4493 g/mol; and LL-37: 37 an, 4514 g/mol). Aβ42 has a net negative charge of −3 (discounting the charge of histidines), while LL-37 has a positive charge of +6, providing for strong electrostatic attraction. Aβ has four aromatic residues (F or Y) with a binary “FF” motif within the KLVFF sequence known to be necessary for Aβ's assembly. [REF: Molecules that target beta-amyloid. Stains C I, Mondal K, Ghosh I. ChemMedChem. 2007 December; 2(12):1674-42. Review. Pubmed ID PMID: 17952881] LL-37 has four aromatic residues as well, and comprises the same “FF” motif within residues 4-8 (DFFRK). LL-37 has nine hydrophobic, aliphatic residues (L, V, I) while Aβ has eleven, again making them biophysically similar. And like Aβ, LL-37 associates into homo-oligomers when alone in solution. [REF: Kinetic characterization of amyloid-beta 1-42 aggregation with a multimethodological approach. Bartolini M, Naldi M, Fiori J, Valle F, Biscarini F, Nicolau D V, Andrisano V. Anal Biochem. 2011 Jul. 15; 414(2).215-25. Pubmed ID PMID 21435333] and [REF: Structure and organization of the human antimicrobial peptide LL-37 in phospholipid membranes, relevance to the molecular basis for its non-cell-selective activity. Oren Z, Lerman J C, Gudmundsson G H, Agerberth B, Shai Y. Biochem J 1999 Aug. 1; 341(Pt 3):501-13. Pubmed ID PMID 10417311] and [REF: New aspects of the structure and mode of action of the human cathelicidin LL-37 revealed by the intrinsic probe p-cyanophenylalanine. Xhindoli D, Morgera F, Zinth U, Rizzo R, Pacor S, Tossi A. Biochem J. 2015 Feb. 1; 465(3):443-57. Pubmed ID PMID 2S378136] When dissolved in aqueous solution, LL-37 peptides may self-assemble, but remain helical in character, unlike Aβ which slowly self-assembles via adoption of β-type secondary structure when alone in solution.

In Lee et al. in 2015, it was reported that the human cathelicidin precursor protein hCAP18 and its downstream cleavage product, the peptide LL-37, are both expressed in many organs of the human body, with the highest basic levels of LL-37 being expressed in the gastrointestinal tract and the brain [REF: Human antimicrobial peptide LL-37 induces glial-mediated neuroinflammation. Lee M, Shi Xi, Barron A E, McGeer E, McGeer P L. Biochem Pharmacol. 2015 Mar. 15; 94(2):130-41. Pubmed ID PMID 25686659] Prior to that 2015 Lee et al. publication, LL-37's expression and functional role in the human central nerve system (CNS) had not been reported. Thus, LL-37 has been proven to be expressed in brain tissue in the human body, as is the Aβ peptide. However, that 2015 publication purported to show, only, that LL-37 might be pro-inflammatory in human brain tissue, like Aβ itself. That 2015 paper did not mention or suggest any possibility of a binding interaction between LL-37 and Aβ.

### Results of the SPRi Interaction Study of LL-37:Aβ Interaction

The physical interaction and binding specificity between LL-37 and different Aβ forms was demonstrated by SPRi, taking advantage of the simultaneous monitoring of different interactions on the same chip enabled by the imaging configuration of the technique CCD images and related plasmonic curves (FIGS. 4-9) correlated to the amount of molecules adsorbed on the surface, showed an increasing immobilization density from low-MW oligomers to high-MW oligomers. [REF: Kinetic characterization of amyloid-beta 1-42 aggregation with a multimethodological approach. Bartolini M, Naldi M, Fiori J, Valle F, Biscarini F, Nicolau D V, Andrisano V. Anal Biochem. 2011 Jul. 15; 414(2):215-25. Pubmed ID PMID 21435333] and [REF: Multifunctional cholinesterase and amyloid Beta fibrillization modulators. Synthesis and biological investigation. Butini S, Brindisi M, Brogi S, Maramai S, Guarino E, Panico A, Saxena A, Chauhan V, Colombo R, Verga L, De Lorenzi E, Bartolini M, Andrisano V, Novellino E, Campiani G, Gemma S. ACS Med Chem Lett. 2013 Oct. 6; 4(12):1178-82. Pubmed ID PMID 24900626]. While SPR measurements revealed higher absolute SPRi signals (Rₘₐₓ) for Aβ with oligomeric features, the affinity for LL-37 was higher for the low-MW oligomeric forms present at the early stage of aggregation (i.e., Aβ40 (t=0 days) and Aβ42 (t=0 days)). [REF: Kinetic characterization of amyloid-beta 1-42 aggregation with a multimethodological approach. Bartolini M, Naldi M, Fiori J, Valle F, Biscarini F, Nicolau D V, Andrisano V. Anal Biochem. 2011 Jul. 15; 414(2):213-25. Pubmed ID PMID 21435333] and [REF: Capillary electrophoresis studies on the aggregation process of beta-amyloid 1-42 and 1-40 peptides. Sabella S, Quaglia M, Lanni C, Racchi M, Govoni S, Caccialanza G, Calligaro A, Bellotti V, De Lorenzi E. Electrophoresis. 2004 October; 25(18-19).3186-94. Pubmed ID PMID 15472964]

Several studies have reported the use of conventional SPR approaches to study Aβ potential interactors. Kai et al. reported the inhibition of Aβ fibril formation by tabersonine and demonstrated the binding between this small molecule and Aβ42 monomers and oligomers immobilized on the SPR chip surface. [REF: Tabersonine inhibits amyloid fibril formation and cytotoxicity of Aβ(1-42). Kai T, Zhang L, Wang X, Jing A, Zhao B, Yu X, Zheng J, Zhou F. ACS Chem Neurosci. 2015 Jun. 17; 6(6).879-48. Pubmed ID PMID 25874995] Equilibrium binding studies yield K_{D} values of 69 and 535 μM for Aβ42 monomers and oligomers, respectively. In a similar study, Barr et al verified the inhibition of Aβ fibril formation by a 15-amino acid peptide [REF: Validation and Characterization of a Novel Peptide That Binds Monomeric and Aggregated β-Amyloid and Inhibits the Formation of Neurotoxic Oligomers Barr R K, Verdile G, Wijaya L K, Morici M, Taddei K, Gupta V B, Pedriní S, Jin L, Nicolazzo J A, Knock E, Fraser P E, Martins R N. J Biol Chem. 2016 Jan. 8; 291(2):547-59. Pubmed ID PMID 26538562] SPR surfaces functionalized with monomeric, oligomeric and fibrillar Aβ42 revealed a stronger affinity of the polypeptide toward the fibrillar form, although only a K_{D} value of 11 μM was measured by injecting Aβ42 onto immobilized peptides and no affinity constant was given. In the SPRi studies described above, kinetic constants in the range between 8 to 20 μM were found, similar to what is published for other Aβ fibril inhibitors as noted above. On the other hand, as mentioned earlier (and differently to what was previously shown) [REF: Tabersonine inhibits amyloid fibril formation and cytotoxicity of Aβ(1-42). Kai T, Zhang L, Wang X, Jing A, Zhao B, Yu X, Zheng J, Zhou F. ACS Chem Neurosci. 2015 Jun. 17; 6(6):879-88. Pubmed ID PMID 25874995] and [REF: Validation and Characterization of a Novel Peptide That Binds Monomeric and Aggregated β-Amyloid and Inhibits the Formation of Neurotoxic Oligomers. Barr R K, Verdile G, Wijaya L K, Morici M, Taddei K, Gupta V B, Pedrini S, Jin L, Nicolazzo J A, Knock E, Fraser P E, Martins R N. J Biol Chem. 2016 Jan. 8; 291(2):547-59. Pubmed ID PMID 26538562], LL-37 shows higher affinity for low-MW Aβ oligomers. This strongly supports the ability of LL-37 to inhibit fibril formation by shining the equilibrium toward smaller species of Aβ, according to the fibrillization mechanisms proposed by Cohen et al. [REF: Proliferation of amyloid-β42 aggregates occurs through a secondary nucleation mechanism. Cohen S I, Linse S, Luheshi L M, Hellstrand E, White D A, Rajah L, Otzen D E, Vendruscolo M, Dobson C M, Knowles T P. Proc Natl Acad Sci USA. 2013 Jun. 11; 110(24):9758-63. Pubmed ID PMID 23703910]

### Effects of LL-37 and Aβ on Cytotoxicity and Inflammatory Cytokine Release

The results of the MTT assays showed that Aβ42 and LL-37 both induced microglial-mediated toxicity to SH-SY5Y cells. At the same time, the results of quantitative ELISA assays on IL-6 and TNFα showed that the levels of these proinflammatory makers released from microglia were markedly elevated compared with that of the untreated control group, when each peptide was added alone. However, the effects of each peptide on cell viability and inflammatory marker production were largely mitigated when the two peptides were co-incubated prior to being added to the culture medium. These data support the hypothesis that Aβ42 interacts with LL-37 as seen by the considerable attenuation of the toxicity of the peptides when they are in complexation.

Certain aspects of our results am not surprising, as previously we have shown that microglia, upon stimulation by LL-37, can release proinflammatory cytokines that affect neurons. [REF: Human antimicrobial peptide LL-37 induces glial-mediated neuroinflammation. Lee M, Shi X I, Barron A E, McGeer E, McGeer P L. Biochem Pharmacol. 2015 Mar. 15; 94(2):130-41. Pubmed ID PMID 25686659] Similarly, Aβ has been shown to activate microglia to produce inflammatory cytokines [REF: Microglia-mediated neurotoxicity: uncovering the molecular mechanisms. Block M L I, Zecca L, Hong J S. Nat Rev Neurosci. 2007 January; 8(1):57-69. Pubmed ID PMID 17180163] However, an attenuation of the release of proinflammatory factors (IL-6 and TNFα) upon treatment of microglia with equimolar LL-37 and Aβ peptide mixtures is a novel finding, which strongly supports our finding that these two peptides may be natural binding partners, which perhaps the body seeks to maintain in balance over the longer term (after an acute innate immune response). Unlike either peptide alone, which causes human microglia to release proinflammatory and neurotoxic cytokines, the complex of the two peptides lacks that neurotoxic effect.

### Correlation of Biophysical Activities of Aβ Peptides and LL-37

Prior to this new work, no one has established a correlation between cathelicidin expression levels and Alzheimer's disease. However, in addition to those mentioned above, other literature reports suggest that the biophysical activities and signaling functions of Aβ peptides and LL-37 are related in vivo. For instance, the Formyl-like Peptide Receptor 1 (FPRL1) is activated by both Aβ [REF: Amyloid (beta)42 activates a G-protein-coupled chemoattractant receptor, FPR-like-1. Le Y, Gong W, Tiffany H L, Tumanov A, Nedospasov S, Shen W, Dunlop N M, Gao X, Murphy P M, Oppenheim J J, Wang J M. J Neurosci. 2001 Jan. 15; 21(2):RC123. Pubmed ID PMID 11160457] and LL-37 [REF: LL-37, the neutrophil granule- and epithelial cell-derived cathelicidin, utilizes formyl peptide receptor-like 1 (FPRL1) as a receptor to chemoattract human peripheral blood neutrophils, monocytes, and T cells. De Yangl, Chen Q, Schmidt A P, Anderson G M, Wang J M, Wooters J, Oppenheim J J, Chertov O. J Exp Med. 2000 Oct. 2; 192(7):1069-74. Pubmed ID PMID 11015447]. This receptor is reportedly involved in inflammatory aspects of AD [REF: Potential role of the formyl peptide receptor-like 1 (FPRL1) in inflammatory aspects of Alzheimer's disease. Cui Y, Le Y, Yazawa H, Gong W, Wang J M. J Leukoc Biol. 2002 October; 72(4):628-35. Review. Pubmed ID PMID: 12377930] via its effects on phagocyte responses [REF: Role of formyl peptide receptor-like 1 (FPRL1/FPR2) in mononuclear phagocyte responses in Alzheimer disease. Iribarren P, Zhou Y, Hu J, Le Y, Wang J M. Immunol Res. 2005; 31(3):165-76. Review. Pubmed PMID: 15888909]; and a decreased phagocyte clearance of CNS β-amyloid is a hallmark of AD [REF: Decreased clearance of CNS beta-amyloid in Alzheimer's disease. Mawuenyega K G I, Sigurdson W, Ovod V, Munsell L, Kasen T, Morris J C, Yarasheski K E, Bateman R J. Science. 2010 Dec. 24; 330(6012):1774. Pubmed ID PMID 21143344] As mentioned above, LL-37 itself is involved in monocyte and macrophage autophagy; its expression, stimulated by Vitamin D3 [REF: Human cathelicidin antimicrobial peptide (CAMP) gene is a direct target of the vitamin D receptor and is strongly up-regulated in myeloid cells by 1,25-dihydroxyvitamin D3. Gombart A F, Borregaard N, Koeffler H P. FASEB J. 2005 July; 19(9):1067-77. Pubmed ID PMID 15985530], engenders enhanced autophagy [REF: Vitamin D3 induces autophagy in human monocytes/macrophages via cathelicidin Yuk J M I, Shin D M, Lee H M, Yang C S, Jin H S, Kim K K, Lee Z W, Lee S H, Kim J M, Jo E K Cell Host Microbe. 2009 Sep. 17; 6(3):23143. Pubmed ID PMID 19748465]. Thus, a dearth of LL-37 generally would reduce phagocytic activity. Phenylbutyrate and Vitamin D3 have each, individually, been shown to be neuroprotective in Alzheimer's Disease mouse models [REF: 1α,25-Dihydroxyvitamin D3 reduces cerebral amyloid-β accumulation and improves cognition in mouse models of Alzheimer's disease. Durk M R, Han K, Chow E C, Ahrens R, Henderson J T, Fraser P E, Pang K S. J Neurosci. 2014 May 21; 34(21):7091-7101. Pubmed ID PMID 24849345] and [REF: Cur Pharm Des. 2013; 19(28):5076-84. Phenylbutyrate is a multifaceted drug that exerts neuroprotective effects and reverses the Alzheimer's disease-like phenotype of a commonly used mouse model. Cuadrado-Tejedor M, Ricobaraza A L, Torrijo R, Franco R Garcia-Osta A. Pubmed ID PMID 23448463], and these same two compounds are known cathelicidin inducers, although this connection between cathelicidin induction and Alzheimer's Disease has not been noticed prior to this patent [REF: Phenylbutyrate induces antimicrobial peptide expression. Steinmann J, Halldórasson S, Agerberth B, Gudmundsson G H. Antimicrob Agents Chemother 2009 December; 53(12):5127-33. Pubmed ID PMID 19770273]. Finally, there is mounting evidence that innate immunity plays a larger role in Alzheimer's disease than previously thought, and that Aβ's normal function may be as an antimicrobial and antiviral peptide [REF: The Alzheimer's disease-associated amyloid beta-protein is an antimicrobial peptide. Soscia S J, Kirby J E, Washicosky K J, Tucker S M, Ingelsson M, Hyman B, Burton M A, Goldstein L E, Duong S, Tanzi R E, Moir R D. PLoS One. 2010 Mar. 3; 5(3):e9505. Pubmed ID PMID: 20209079] and [REF: Sci Transl Med. 2016 May 25; 8(340):340ra72. Amyloid-β peptide protects against microbial infection in mouse and worm models of Alzheimer's disease. Kumar D K, Choi S H, Washicosky K J, Eimer W A, Tucker S, Ghofrani J, Lefkowitz A, McColl G, Goldstein L E, Tanzi R E, Moir R D. Pubmed ID PMID 27225182] and [REF: Biogerontology. 2015 February; 16(1):85-98. β-Amyloid peptides display protective activity against the human Alzheimer's disease-associated herpes simplex virus-1. Bourgade K, Garneau H, Giroux G, Le Page A Y, Bocti C, Dupuis G, Frost E, Fülöp T Jr. Pubmed ID PMID 25376108] and [REF: Alzheimer's Disease-Associated β-Amyloid Is Rapidly Seeded by Herpesviridae to Protect against Brain Infection. Eimer W A, Kumar D K V, Shanmugam N K N, Rodriguez A S, Mitchell T, Washicosky K J, György B, Breakefield X O, Tanzi R E, Moir R D. Neuron. 2018 Jul. 11; 99(1):56-63]. Kumar et al. showed that Aβ expression protects against fungal and bacterial infections in mouse, nematode, and cell culture models of AD [REF: Sci Transl Med. 2016 May 25; 9(340):340ra72. Amyloid-β peptide protects against microbial infection in mouse and worm models of Alzheimer's disease. Kumar D K, Cho S H, Washicosky K J, Eimer W A, Tucker S, Ghofrani J, Lefkowitz A, McColl G, Goldstein L E, Tanzi R E, Moir R D. Pubmed ID PMID 27225182]. And, part of the body's natural response to infection is, of course, to upregulate cathelicidin [REF: The chemistry and biology of LL-37. Burton M F, Steel P G. Nat Prod Rep. 2009 December, 26(12):1572-84. Review. Pubmed ID PMID: 19936387].

### Significance of Studies of LL-37:Aβ Interaction and Binding In Vitro

The work described above demonstrates that these two peptides, the cathelicidin peptide LL-37 and the Alzheimer's Disease-associated peptide Aβ, which are both ubiquitous in human tissues and expressed in the human brain, bind to and interact with each other in different aggregation states, with affinity constants typical of protein-protein interactions and close to values reported in the literature for the binding of Aβ with optimized synthetic peptides. The addition of LL-37 to Aβ42, in a buffer that mimics quasi-physiological conditions, strongly inhibits fibril formation. The protective effect of LL-37's presence against microglia-mediated Aβ42 toxicity to SH-SY5Y neuroblastoma cells is an important new finding, revealing that in combination the two peptides are 90% less pro-inflammatory than either peptide alone. Literature reports suggest that numerous biophysical activities and signaling functions of Aβ peptides and LL-37 are related in vivo. The in vitro data presented here were the starting point from which we are further investigating whether factors that affect cathelicidin gene CAMP regulation, which will in turn control the production of LL-37 in vivo, modulate Aβ aggregation and/or microglia-induced neuroinflammation.

### Correlations Between Infections of Brain Tissue, and Alzheimer's Disease

As has been mentioned above, it has been hypothesized that the Alzheimer's Disease-associated peptide Aβ may itself be an important host defense peptide. However, it is not yet well understood what causes the expression of the Aβ peptide or its precursor protein, APP. [REF: The Alzheimers disease-associated amyloid beta-protein is an antimicrobial peptide. Soscia S J, Kirby J E, Washicosky K J, Tucker S M, Ingelsson M, Hyman B, Burton M A, Goldstein L E, Duong S, Tanzi R E, Moir R D. PLoS One. 2010 Mar. 3; 5(3):e9505. Pubmed ID PMID: 20209079] and [REF: Sci Transl Med. 2016 May 25, 8(40):340ra72. Amyloid-β peptide protects against microbial infection in mouse and worm models of Alzheimer's disease. Kumar D K, Choi S H, Washicosky K J, Eimer W A, Tucker S, Ghofrani J, Lefkowitz A, McColl G, Goldstein L E, Tanzi R E, Moir R D. Pubmed ID PMID 27225182] and [REF: Biogerontology. 2015 February; 16(1):85-98. β-Amyloid peptides display protective activity against the human Alzheimers disease-associated herpes simplex virus-1. Bourgade K, Gameau H, Giroux G, Le Page A Y, Bocti C, Dupuis G, Frost E H, Fülöp T Jr. Pubmed ID PMID 253761081 Kumar et al. showed that Aβ expression protects against fungal and bacterial infections in mouse, nematode, and cell culture models of AD [REF: Sci Transl Med. 2016 May 25, 8(340):340ra72. Amyloid-β peptide protects against microbial infection in mouse and worm models of Alzheimer's disease. Kumar D K, Choi S H, Washicosky K J, Eimer W A, Tucker S, Ghofrani J, Lefkowitz A, McColl G, Goldstein L E, Tanzi R E, Moir R D. Pubmed ID PMID 27225182]. It has been shown that infection of brain tissue with Herpesvirus results in rapid expression of Aβ peptide, which then entraps the Herpes viral particles in beta-amyloid, which may mean that viral infections actually stimulate the expression of Aβ. [REF: Alzheimer's Disease-Associated β-Amyloid Is Rapidly Seeded by Herpesviridae to Protect against Brain Infection. Eimer W A, Kumar D K V. Shanmugam N K N, Rodriguez A S, Mitchell T, Washicosky K J, György B, Breakefield X O, Tanzi R E, Moir R D. Neuron. 2018 Jul. 11; 99(1):56-43. Pubmed ID PMID 30001512]. Other pathogens, besides Herpesvirus, which seem to infect brain tissue and correlate with higher risk of Alzheimer's Disease include the bacterial pathogens Chlamydia pneumoniae and the family of Spirochete bacteria. [REF: Historic evidence to support a causal relationship between spirochetal infections and Alzheimer's disease. Miklossy J. Front Aging Neurosci. 2015 Apr. 16; 7:46. Pubmed ID PMID 25932012] and [REF: Chlamydia pneumoniae infection of monocytes in vitro stimulates innate and adaptive immune responses relevant to those in Alzheimer's disease. Lim C, Hammond C J, Hingley S T, Balin B J. J Neuroinflammation. 2014 Dec. 24; 11:217. Pubmed ID PMID: 25540075] and [Bacterial Amyloid and DNA are Important Constituents of Senile Plaques: Further Evidence of the Spirochetal and Biofilm Nature of Senile Plaques. Miklossy J. J Alzheimers Dis. 2016 Jun. 13; 53(4):1459-73. Pubmed ID 27314530] A meta-analysis of all studies that have examined the relationship between bacterial infections of brain tissue, and Alzheimer's Disease (AD), found over a ten-fold increased occurrence of AD when there was detectable evidence of spirochetal infection (OR: 10.61; 95% CI: 3.38-33.29), with over a four-fold increased occurrence of AD as a conservative risk estimate (OR 4.45; 95% CI: 2.33-8.52). The same meta-analysis also found over a five-fold increased occurrence of AD with Chlamydophila pneumoniae infection (OR: 5.66; 95% CI: 1.83-17.51). Therefore, there is a strongly positive association between bacterial infection of the brain and AD. [REF: Bacterial infection and Alzheimer's disease: a meta-analysis. Mabeshwari P, Eslick G D. J Alzheimers Dis. 2015; 43(3).957-66. Pubmed ID PMID 25192736] If indeed, as these studies indicate, Aβ is a host defense peptide just as LL-37 is, then their role as binding partners that can detoxify each other also makes sense. Also, it should be noted that the LL-37 peptide has been shown to be active against both HSV-1 and HSV-2, two different strains of Herpesvirus against which it has been tested. [REF: Hunan cathelicidin (LL-37), a multifunctional peptide, is expressed by ocular surface epithelia and has potent antibacterial and antiviral activity. Gordon Y J I, Huang L C, Romanowski E G, Yates K A, Proske R J, McDermott A M. Cur Eye Res 2005 May; 30(5):385-94. Pubmed ID PMID 16020269] and [REF: Cathelicidin deficiency predisposes to eczema herpeticum. Howell M D, Wollenberg A, Gallo R L, Flaig M, Streib J E, Wong C, Pavicic T, Boguniewicz M, Leung D Y. J Allergy Clin Immunol. 2006 April; 117(4):836-41. Pubmed ID PMID 16630942] interestingly, the particular bacterial pathogen Chlamydia pneumoniae, which is associated with AD, appeared in one study to resist the antibacterial activity of LL-37 peptide. [REF: Involvement of the antimicrobial peptide LL-37 in human atherosclerosis. Edfeldt K, Agerberth B, Rottenberg M E, Gudmundsson G H, Wang X B, Mandal K, Xu Q, Yan Z Q. Arterioscler Tromb Vasc Biol. 2006 July; 26(7):1551-7. Pubmed ID PMID 1664514] Finally, a 2018 study of the brains of patients with Alzheimer's Disease found increased prevalence of human herpesvirus 6A (HHV-6A) and human herpesvirus 7 (HHV-7) within the brain tissue of subjects with Alzheimer's Disease, compared with controls. These results were replicated in two additional, independent and geographically dispersed cohorts. [REF: Multiscale Analysis of Independent Alzheimer's Cohorts Finds Disruption of Molecular, Genetic, and Clinical Networks by Human Herpesvirus. Readhead B, Haure-Mirande J V, Funk C C, Richards M A, Shannon P, Haroutunian V, Sano M, Liang W S, Beckmann N D, Price N D, Reiman E M, Schadt E E, Ehrlich M E, Candy S, Dudley J T. Neuron. 2018 Jul. 11; 99(1):64-82.e7. Pubmed ID PMID 29937276] Whether this statistically significant, increased burden of Herpesviridae in the brains of Alzheimer's Disease sufferers is causative of neurodegeneration, or correlative with the disease process, is not yet clear: however, this finding is important. If, as put forward by Moir and Tanzi et al., Aβ amyloid oligomers, fibrils and plaques serve to sequester and inactivate Herpesvirus particles, then LL-37-stimulated macroautophagy may serve to clear away these molecular debris from the brain, once the virus has been rendered inactive by immobilization in AP plaques. [REF: Vitamin D3 induces autophagy in human monocytes/macrophages via cathelicidin Yuk J M, Shin D M, Lee H M, Yang C S, Jun H S, Kim K K, Lee Z W, Lee S H, Kim J M, Jo E K. Cell Host Microbe. 2009 Sep. 17; 6(3):231-43. Pubmed ID PMID: 19748465]

### Inducer of Cathelicidin Gene, CAMP, and Obligate Co-Factors for CAMP Expression

There are a number of known inducers and essential cofactors for expression of the cathelicidin gene, which is called CAMP in humans, and cup in mice. One important compound for the induction of CAMP gene expression is Vitamin D3. [REF: Human cathelicidin antimicrobial peptide (CAMP) gene is a direct target of the vitamin D receptor and is strongly up-regulated in myeloid cells by 1,25-dihydroxyvitamin D3. Gombart A F I, Borregaard N, Koeffler H P, FASEB J. 2005 July; 19(9):1067-77. Pubmed ID PMID 15985530] and [REF: Vitamin D as an inducer of cathelicidin antimicrobial peptide expression: past, present and future. White J H. J Steroid Biochem Mol Biol. 2010 July; 121(1-2):234-8. Pubmed ID PMID 20302931] Vitamin D3 is a particularly potent inducer of cathelicidin in human immune cells. [REF: Regulation of the human cathelicidin antimicrobial peptide gene by 1α,25-dihydroxyvitamin D3 in primary immune cells. Lowry M B, Guo C, Borregaard N, Gombart A F. J Steroid Biochem MCA Biol. 2014 September; 143:183-91. Pubmed PMID: 24565560] Interestingly, this Vitamin D3 requirement for CAMP expression in human beings and primates, especially, is related to the existence of a Vitamin D Receptor (VDR) binding domain in transcriptional control region of the gene. [REF: Exaptation of an ancient Alu short interspersed element provides a highly conserved vitamin D-mediated innate immune response in humans and primates. Gombart A F, Saito T, Koeffler H P. BMC Genomics. 2009 Jul. 16; 10:321. doi: 10.1186/1471-2164-10-321. Pubmed ID PMID 19607716] It has been shown that the regulation of cathelicidin expression by Vitamin D3 differs, in different tissues of the body. [REF: Regulation of the CAMP gene by 1,25(OH)2D3 in various tissues. Gombart A F, O'Kelly J, Saito T, Koeffler H P. J Steroid Biochem Mol Biol 2007 March; 103(3-5):552-7. Pubmed ID PMID: 17368184] There is also a Retinoid X Receptor (RXR) binding domain in the CAMP transcriptional control region, which makes RXR agonists obligate cofactors for CAMP gene expression, along with Vitamin D3. RXR agonists include, for instance, natural molecules such as Retinoic Acid (Vitamin A), which is a metabolic by-product of the dietary retinoid β-Carotene, and/or Dodecahexanoic Acid (DHA). A potent non-natural RXR agonist is the compound Bexarotene, which is an FDA-approved drug for cancer treatment. [REF: Vitamin D-induced up-regulation of human keratinocyte cathelicidin antimicrobial peptide expression involves retinoid X receptor α. Svensson D, Nebel D, Voss U, Ekblad E, Nilsson B O. Cell Tissue Res. 2016 November; 366(2):353-362. June 30. Pubmed ID PMID 27357804] and [REF: Label-free quantitative mass spectrometry reveals novel pathways involved in LL-37 expression. Cederlund A, Nylén F, Miraglia E, Bergman P, Gudmundsson G H, Agerberth B. J Innate Immun. 2014; 6(3):365-76. doi: 101159/000355931 Epub 2013 Nov. 16. Pubmed PMID. 24246949]

The use of the RXR agonist DHA in combination with Bexarotene shows synergy and more efficient activation of Retinoid X Receptors, such that a lower dosage of the FDA-approved drug Bexarotene can be used as an RXR agonist, reducing any potential toxicity to liver or kidneys. [REF: Omega-3 Fatty Acids Augment the Actions of Nuclear Receptor Agonists in a Mouse Model of Alzheimer's Disease. Casali B T, Corona A W, Mariani M M, Karlo J C, Ghosal K, Landreth G E. J Neurosci. 2015 Jun. 17; 35(24):9173-81. Pubmed ID PMID 26005639]

In addition to Vitamin D3, certain short-chain fatty acids are able to induce the expression of the CAMP gene, especially Butyrate. [REF: Expression of the cathelicidin LL-37 is modulated by short chain fatty acids in colonocytes. relevance of signalling pathways. Schauber J, Svanholm C, Termen S, Iffland K, Menzel T, Scheppach W, Melcher R, Agerberth B, Lührs H, Gudmundsson G H. Gut. 2003 May; 52(5):735-41. Pubmed ID PMID: 12692061] However, Butyrate is an undesirable therapeutic compound, as it has a noxious smell. As a replacement for Butyrate induction of CAMP gene expression, it was discovered the Phenylbutyrate is highly effective; and does not smell bad. [REF: Phenylbutyrate induces antimicrobial peptide expression. Steinmann J, Halldörason S, Agerberth B, Gudmundsson G H. Antimicrob Agents Chemother. 2009 December; 53(12):3127-33. doi: 10.1128/AAC.00818-09. Epub 2009 Sep. 21. Pubmed ID PMID: 19770273] The combination of Vitamin D3 and Phenylbutyrate is synergistic in its ability to induce CAMP gene expression, providing enhanced expression of LL-37 and antibacterial activity, relative to what can be achieved with just one of the two compounds. [REF: Vitamin D and phenylbutyrate promote development of a human dendritic cell subset displaying enhanced antimicrobial properties. van der Does A M, Kenne E, Koppelaar E. Agerberth B, Lindbom L. J Leukoc Biol 2014 June, 95(6):883-91. Pubmed ID PMID: 24550524] In the United States, the compound Phenylbutyrate is an FDA-approved drug for the treatment of Urea-Cycle Disorders. [REF: Profile of sodium phenylbutyrate granules for the treatment of urea-cycle disorders. patient perspectives. Peña-Quintana L, Llarena M, Reyes-Suárez D, Aldámiz-Echevarría L. Patient Prefer Adherence. 2017 Sep. 6; 11:1489-1496. Review Pubmed ID PMID: 23919721] It has been shown that Phenylbutyrate induction of CAMP gene also occurs via interaction with the Vitamin D receptor, like Vitamin D3 itself [REF: Phenylbutyrate induces cathelicidin expression via the vitamin D receptor: Linkage to inflammatory and growth factor cytokines pathways. Kulkarni N N, Yi Z, Huebnken C, Agerberth B, Gudmundsson G H. Mol Immunol. 2015 February; 63(2).530-9. Pubmed ID PMID: 25458314] The synergistic induction of the CAMP gene by the combination of Vitamin D3 and Phenylbutyrate has been developed as a novel treatment for bacterial infections, for instance, for the treatment of tuberculosis. A dosage study was done in humans, and the ideal dose for induction of cathelicidin for the purpose of treating lung infection was 5000 IU Vitamin D3 taken daily, plus 500 mg Phenylbutyrate per dose taken twice per day. [REF: Induction of the human cathelicidin LL-37 as a novel treatment against bacterial infections van der Does A M, Bergman P, Agerberth B, Lindbom L. J Leukoc Biol. 2012 October; 92(4):735-42. Review. Pubmed ID PMID: 22701042] and [REF: Oral intake of phenylbutyrate with or without vitamin D3 upregulates the cathelicidin LL-37 in human macrophages: a dose finding study for treatment of tuberculosis. Mily A, Rekha R S, Kamal S M, Akhtar E, Sarker P, Rahim Z, Gudmundsson G H, Agerberth B, Raqib R. BMC Pulm Med. 2013 Apr. 16; 13:23. doi: 10.1186/1471-2466-13-23. Pubmed ID PMID: 2359070] and [REF: Significant Effects of Oral Phenylbutyrate and Vitamin D3 Adjunctive Therapy in Pulmonary Tuberculosis: A Randomized Controlled Trial. Mily A, Rekha R S, Kamal S M, Arifuzzaman A S, Rahim Z, Khan L, Haq M A, Zaman K, Bergman P, Brighenti S, Gudmundsson G H, Agerberth B, Raqib R. PLoS One. 2015 Sep. 22; 10(9):e0138340. Pubmed ID PMID: 26394045] and [REF: Immune responses in the treatment of drug-sensitive pulmonary tuberculosis with phenylbutyrate and vitamin D3 as host directed therapy. Rekha R S, Mily A, Sultana T, Haq A, Ahmed S, Mostafa Kamal S M, van Schadewijk A, Hiemstra P S, Gudmundsson G H, Agerberth B, Raqib R. BMC Infect Dis 2018 Jul. 4; 18(1).303. doi: 10.1186/s12879-018-3203-9. Pubmed ID PMID. 29973153] Phenylbutyrate has also been tested in a preclinical animal trial, using rabbits, for the treatment of enteropathogenic E. coli-induced diarrhea, with good success. [REF: Treatment with phenylbutyrate in a pre-clinical trial reduces diarrhea due to enteropathogenic Escherichia coli link to cathelicidin induction. Al-Mamun A, Mily A, Sarker P, Tiash S, Navarro A, Akter M, Talukder K A, Islam M F, Agerberth B, Gudmundsson G H, Cravioto A, Raqib R. Microbes Infect. 2013 November; 15(13):939-50. Pubmed ID PMID: 24016414]

Other compounds have also been shown, more recently, to induce the expression of the CAMP gene, but by Vitamin D3 receptor-independent mechanisms that are not fully understood. For instance, the compound Curcumin induces CAMP gene expression by a non-Vitamin D3 receptor dependent mechanism. [REF: Curcumin induces human cathelicidin antimicrobial peptide gene expression through a vitamin D receptor-independent pathway Guo C, Rosoha E, Lowry M B, Borregaard N, Gombart A F. J Nutr Biochem 2013 May; 24(5):754-9. Pubmed ID PMID: 22841393] Additionally, compounds from the family of stilbenoids, in particular Resveratrol or Pterostilbene, also induce CAMP gene expression by a Vitamin D3 receptor-independent mechanism, which is also synergistic with Vitamin D3. [REF: Synergistic induction of human cathelicidin antimicrobial peptide gene expression by vitamin D and stilbenoids Guo C, Sinnott B, Niu B, Lowry M B, Fantacone M L, Gombart A F. Mol Nutr Food Res. 2014 March; 58(3):528-536. Pubmed ID PMID: 24039193] It has been shown that the manner in which Resveratrol induces cathelicidin expression is by a novel mechanism involving sphingosine-1-phosphate pathway signaling. [REF: Resveratrol stimulates sphingosine-1-phosphate signaling of cathelicidin production. Park K, Elias P M, Hupe M, Borkowski A W, Gallo R L, Shin K O, Lee Y M, Holleran W M, Uchida Y. J Invest Dermatol. 2013 August; 133(8):1942-9. Pubmed ID PMID: 23856934] and [REF: Sphingosine kinase 1 activation enhances epidermal innate immunity through sphingosine-1-phosphate stimulation of cathelicidin production. Jeong S K, Kim Y I, Shin K O, Kim B W, Lee S H, Jean J E, Kim H J, Lee Y M, Mauro T M, Elias P M, Uchida Y, Park K. J Dermatol Sci. 2015 September; 79(3):229-34. Pubmed ID PMID: 26113114] One lipid molecule called Genistein was shown in 2015 also to induce cathelicidin expression by a sphingosine-1-phosphate stimulation mechanism. [REF: The dietary ingredient, genistein, stimulates cathelicidin antimicrobial peptide expression through a novel SIP-dependent mechanism. Park K, Kim Y I, Shin K O, Seo H S, Kim J Y, Mann T, Oda Y, Lee Y M, Holleran W M, Elias P M, Uchida Y. J Nutr Biochem. 2014 July; 25(7).734-40. Pubmed PMID: 24768661] Genistein is a soy (or other plant, such as red clover)-derived isoflavanoid compound that has a multitude of health benefits. [REF: Bioactivity of genistein: A review of in vitro and in vivo studies. Ganai A A, Farooqi H. Biomed Pharmacother. 2015 December; 76:30-8. doi. 10.1016/j.biopha.2015.10.026. Epub 2015 Nov. 10. Review. Pubmed ID PMID: 26653547] Interestingly, Genistein and Resveratrol have been shown to have synergistic anticancer effects (with no reference to cathelicidin gene expression); this highlights that natural compounds of this type often show synergies. [REF: A new class of hybrid anticancer agents inspired by the synergistic effects of curcumin and genistein: Design, synthesis, and anti-proliferative evaluation. Chen Q H, Yu K, Zhang X, Chen G, Hoover A, Leon F, Wang R, Subrahmanyam N, Addo Mekuria E, Harinantenaina Rakotondraibe L. Bioorg Med Chem Lett. 2015 Oct. 15, 25(20):4553-6. doi. 10.1016/j.bmcl.2015.08.064. Epub 2015 Aug. 24. Pubmed ID PMID: 26341135] And again, it should be noted that Curcumin's induction of cathelicidin expression is also by a non-Vitamin D receptor dependent mechanism; but perhaps different from that of Genistein, given their synergistic effects in treating cancer [REF: Curcumin induces human cathelicidin antimicrobial peptide gene expression through a vitamin D receptor-independent pathway. Guo C, Rosoha E, Lowry M B, Borregaard N, Gombart A F. J Nutr Biochem 2013 May; 24(5):754-9. Pubmed ID PMID: 22841393]

In 2016, a novel family of compounds called aroylated phenylenediamines was developed to achieve the potent induction of the cathelicidin gene, which includes the compound Entinostat, for the treatment of infections such as Cholera. [REF: Potent Inducers of Endogenous Antimicrobial Peptides for Hint Directed Therapy of Infections. Ottosson H, Nylén F, Sarker P, Miraglia E, Bergman P, Gudmundsson G H, Raqib R, Agerberth B, Stromberg R. Sci Rep. 2016 Nov. 9, 6.36692. Pubmed ID PMID: 27827460] and [REF: Entinostat up-regulates the CAMP gene encoding LL-37 via activation of STAT3 and HIF-1α transcription factors. Miraglia E, Nylén F, Johansson K, Arnér E, Cebula M, Farmand S, Ottosson H, Stromberg R, Gudmundsson G H, Agerberth B, Bergman P. Sci Rep. 2016 Sep. 16; 6:33274. Pubmed ID PMID 27633343] and [REF Treatment with Entinostat Heals Experimental Cholera by Affecting Physical and Chemical Barrier Functions of Intestinal Epithelia. Sarker P, Banik A. Stromberg R, Gudmundsson G H, Raqib R, Agerberth B. Antimicrob Agents Chemother. 2017 Jun. 27; 61(7). pii: e02570-16. Pubmed ID PMID: 28438947]

The combination of Vitamin D3 and Phenylbutyrate has been shown to be effective for the treatment of infections such as tuberculosis, and the aroylated phenylenediamine Entinostat is able to induce cathelicidin gene expression via activation of STAT3 and HIF-1α transcription factor, again to treat cholera. However, until now, no combination of CAMP-inducing compounds has ever been proposed for the potential treatment of Alzheimer's Disease. This is because it has only recently been discovered that the LL-37 peptide can bind to and detoxify the Alzheimer's Disease-related peptide Aβ. Thus, until 2017 when this patent was filed, no one would have considered CAMP gene induction as a potential therapy for Alzheimer's Disease. [REF: Evidence that the Human Innate Immune Peptide LL-37 may be a Binding Partner of Amyloid-β and Inhibitor of Fibril Assembly. De Lorenzi E, Chiari M, Colombo R, Cretich M, Sola L, Vanna R, Gagni P, Bisceglia F, Morasso C, Lin J S, Lee M, McGeer P L, Barron A E. J Alzheimers Dis. 2017; 59(4):1213-1226. Pubmed ID PMID 28731438]

Levels of cathelicidin in the human body can be measured by quantitative PCR of DNA extracted from white blood cells, by fluorescent ELISA assays of protein levels, or by radioimmunoassays, as disclosed in these two papers: [REF: Oral intake of phenylbutyrate with or without vitamin D3 upregulates the cathelicidin LL-37 in human macrophages: a doe finding study for treatment of tuberculosis. Mily A, Rekha R S, Kamal S M, Akhtar E, Sarker P, Rahim Z, Gudmundsson G H, Agerberth B, Raqib R. BMC Pulm Med. 2013 Apr. 16; 13:23 doi. 10.1186/1471-2466-13-23. Pubmed ID PMID: 23590701] and [REF: PLoS One. 2016 May 6; 11(5):e0152711. Circulating Cathelicidin Concentrations in a Cohort of Healthy Children. Influence of Age, Body Composition, Gender and Vitamin D Status. Stukes T M, Shary J R, Wei W, Ebeling M D, Dezsi K B, Shary F S, Forestieri N E, Hollis B W, Wagner C L. Pubmed ID PMID 27152524]

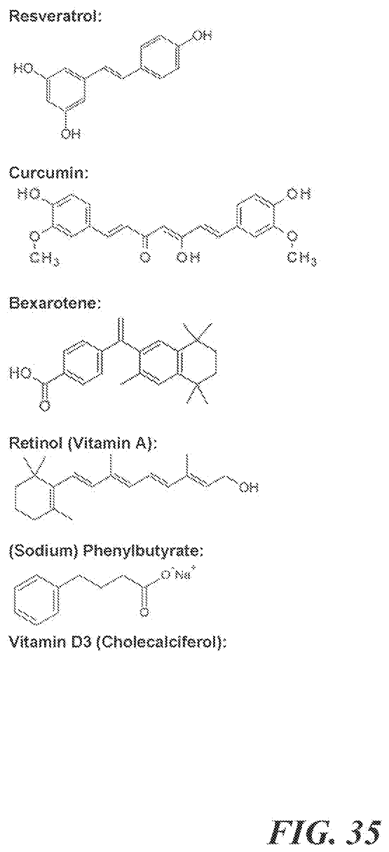

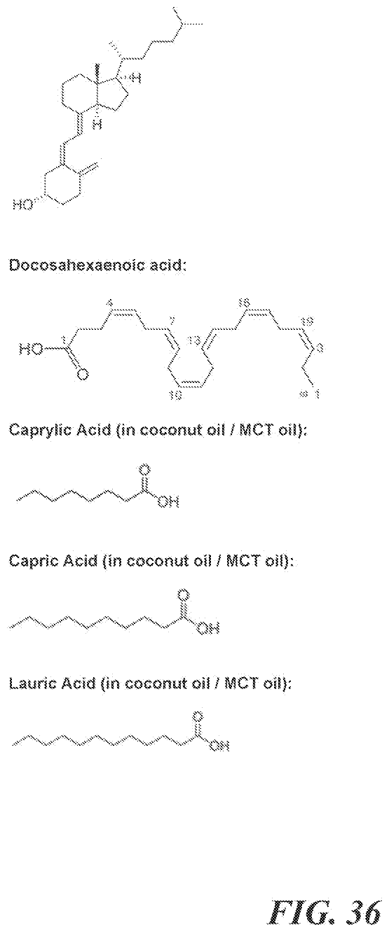

We disclose hem a novel polytherapy, i.e., mixture of a number of different compounds, for the purposes of inducing the expression of the cathelicidin gene CAMP (see FIGS. 35-36). As all of these CAMP-inducing compounds are hydrophobic or lipophilic, and not soluble in water, we dissolved them in liquid coconut oil, or medium-chain triglyceride oil, composed of capric acid, caprylic acid, and lauric acid. The mixture of compounds that we disclose for the synergistic induction of CAMP gene expression, which can have many human health benefits, including the potential treatment of Alzheimer's Disease and other conditions is as follows: Vitamin D3, Phenylbutyrate, Curcumin, Resveratrol, Bexarotene, and DHA, all dissolved in liquid coconut oil (which comprises capric acid, caprylic acid, and lauric acid) (FIGS. 35-36). The rationale for using this mixture of six different compounds, dissolved in coconut oil, is that all of these have been shown to induce LL-37 expression, with a variety of different mechanisms of induction, and with synergy of induction occurring (by different mechanisms), at least for the pair of Vitamin D3 and Phenylbutyrate, and Vitamin D3 and Resveratrol. The other rationale for combining these six compounds are that all of them are proven to be safe for dosing in humans. Vitamin D3, Curcumin, Resveratrol, and DHA are all GRAS (Generally Recognized As Safe) compounds according to the U.S. FDA, whereas both Phenylbutyrate and Bexarotene are already FDA-approved as therapeutic compounds, which means that safe doses have been established. Given that all of these compounds have different structures and degrees of lipophilicity/hydrophilicity (FIG. 35-36), they will each partition differently into different body compartments, and can broaden the mechanism of cathelicidin induction by being used simultaneously.

We have shown that this particular combination of six compounds, dissolved in liquid coconut oil, and orally dosed in mice every other day, can induce the expression of the cathelicidin gene in the brain. The particular dosage used to show this brain induction of cathelicidin expression, for the first time, is shown in TABLE 2 below.

TABLE 2
Dosages for Induction of Cathelicidin
Expression in C57BL/6J Mice

| | Dosage | Dose per avg mouse |
|---|---|---|
| Treatment | (mg/kg) | (mg/two days) |
| Phenylbutyrate | 33.33 | 0.83 |
| Curcumin | 50.00 | 1.25 |
| Bexarotene (Targretin ™) | 8.33 | 0.21 |
| 1,25-dihydroxyvitamin D3 | 5.56E−04 | 1.67E−05 |
| Resveratrol | 41.67 | 1.04 |
| DHA | 66.67 | 1.67 |
| Coconut oil (MCT oil) | | 100 |

Preliminary studies in wild-type (C57BL/6J) mice using the oral polytherapy treatment, dosed as described in the table above, demonstrated significantly increased levels of cathelicidin gene expression in brain tissue comprised to untreated controls. (FIG. 12). In this study, groups of 4-5 wild type mice were dosed every other day, for 2 weeks, with the polytherapy formulation shown in the table. Then, after two weeks of dosing, at particular and exact times after the last dose was given, the mice were sacrificed, and their brain tissue was tested by quantitative PCR for the levels of cathelicidin gene expression. This allowed us to see the time-dependent cathelicidin gene expression after dosing, at 30,60, and 180 minutes after dosing. This polytherapy provided, on average, between 3-fold and 7.5-fold induction of cathelicidin gene expression, within 3 hours. This is the first time that oral induction of cathelicidin gene expression in the brain has ever been shown, this is a new and unpublished result.

We have also tested this oral polytherapy using the 5XFAD mouse model as an inflammatory preclinical model of Alzheimer's disease with an aggressive amyloid beta accumulation pathology, to model cerebral amyloidosis with intracellular amyloid beta and amyloid beta plaques beginning to appear as early as 2 months' age. [REF: Neuron loss in the 5XFAD mouse model of Alzheimer's disease correlates with intraneuronal Aβ342 accumulation and Caspase-3 activation Eimer W A, Vassar R. Mol Neurodegener. 2013 Jan. 14; 8:2. Pubmed ID PMID: 23316765] and [REF: Genetic mouse models of brain ageing and Alzheimer's disease. Bilkei-Gorzo A. Pharmacol Ther. 2014 May; 142(2):244-37. Review. Pubmed ID PMID: 24362083] and [REF: Molecular Differences and Similarities Between Alzheimer's Disease and the 5XFAD Transgenic Mouse Model of Amyloidosis Maarouf C L, Kokjohn T A, Whiteside C M, Macias M P, Kalback W M, Sabbagh M N, Beach T G, Vassar R, Roher A E. Biochem Insights. 2013 Nov. 21; 6:1-10. Pubmed ID PMID: 25210460) Experiments commenced at 5 months age. Mice were dosed with the polytherapy as described in Table 2, every other day, for 70 days total. The 5XFAD transgenic model reliably replicates neuropathology and cognitive symptoms of Alzheimer's disease not present in alternative models. In order to obtain sufficient power in our statistical analysis and taking into account the inherent variability in behavioral testing and stereotaxic injections, we used 14-15 mice in each treatment group as well as in the untreated control groups. We compared four groups: (1) treated and (2) untreated wild type C57BL/6J mice, and (3) treated and (4) untreated 5XFAD mice. Mouse studies were carried out with APLAC and IACUC approval.

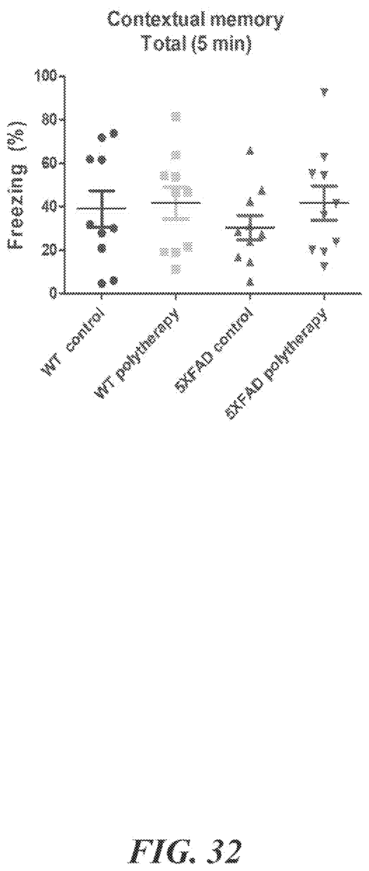

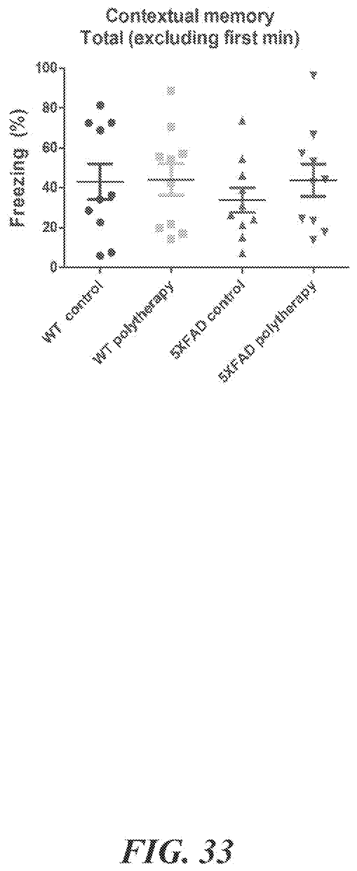

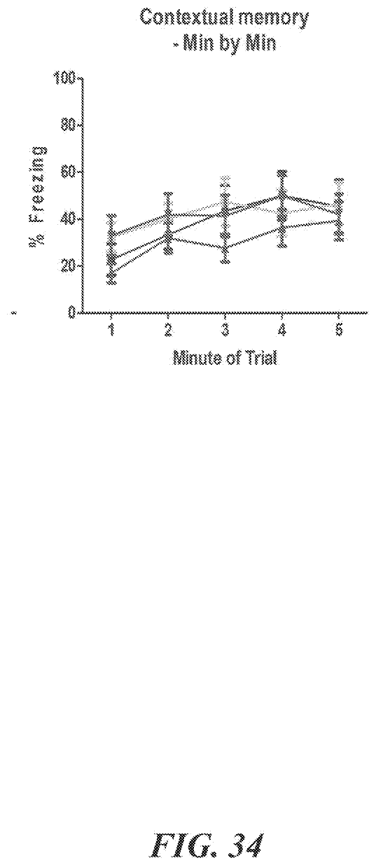

We have completed the dosing portion of the study and are in the process of analyzing the data to look for treatment effects on amyloid plaque loads, inflammation, autophagy and cell senescence. As shown in FIGS. 32-34, the results from behavioral testing are promising (contextual fear conditioning) in which treated 5XFAD mice showed memory comparable to wild type mice, and significantly improved over untreated 5XFAD. This indicates efficacy of this six-component oral polytherapy in treating Alzheimer's Disease, in the well-accepted 5XFAD mouse model of this neurodegenerative disease (unpublished data).

Interestingly, in 2015 it was shown that neutrophils and their creation of Neutrophil Extracellular Traps (NETs) play a role in the inflammatory brain pathology in the 5XFAD mouse model of Alzheimer's Disease. It was found that activated neutrophils could extravasate and cross the blood brain barrier, and release NETs inside brain tissue. [REF: Neutrophils promote Alzheimer's disease-like pathology and cognitive decline via LFA-1 integrin. Zenaro E, Pietronigro E, Della Bianca V, Piacentino G, Marongiu L, Budui S, Turano E, Rosi B, Angiari S, Dusi S, Montresor A, Carlucci T, Nani S, Tosadori G, Calciano L, Catalucci D, Berton G, Bonetti B, Constantin G. Nat Med. 2015 August; 21(8):880-6. Pubmed ID PMID: 26214837] Since, as discussed above, the cathelicidin peptide plays a central role in the clearance of neutrophil NETs, which otherwise can be proinflammatory if left in place, it makes sense that the induction of the camp cathelicidin gene in 5XFAD mice was able to enhance their cognitive function, as shown in FIGS. 32-34, where the treated 5XFAD mice had memory that was equivalent to the wild type mice, when tested in the same fear conditioning experiment.

In a prior study in which CAMP induction with Vitamin D3 and Phenylbutyrate to treat tuberculosis, the proper dosing for humans of Vitamin D3 was found to be 5000 IU, and for Phenylbutyrate was 500 mg per dose, twice per day. [REF: Oral intake of phenylbutyrate with or without vitamin D3 upregulates the cathelicidin LL-37 in human macrophages: a dose finding study for treatment of tuberculosis. Mily A, Rekha R S, Kamal S M, Akhtar E, Sarker P, Rahim Z, Gudmundsson G H, Agerberth B, Raqib R. BMC Pulm Med 2013 Apr. 16; 13:23. doi: 10.1186/1471-2466-13-23. Pubmed ID PMID: 23590701] The FDA-recommended and approved initial dosage of the drug Bexarotene is 300 mg/m²/day. The dosages of the other compounds, Curcumin, Resveratrol, and DHA, could for example be set at typical levels they are provided as GRAS daily supplements: Curcumin, 500 mg per dose, twice per day; Resveratrol, 250 mg per dose, twice per day; and DHA, 500 mg per dose, twice per day. However, since these CAMP-induced compounds are provided in a polytherapy, much lower doses of each or any individual compound may be effective for the intended purpose, in combination with the others.

It is expected that this synergistic combination of compounds that all help induce cathelicidin expression by causing the expression of the LL-37 peptide in the brain. Without wishing to be bound by theory, this is expected to serve as a useful treatment for Alzheimer's Disease by (1) binding to and detoxifying the Alzheimer's Disease-associated peptide Aβ; (2) enhancing the autophagic clearance of Aβ fibrils and plaques in the brain, as well as the macroautophagy of any dead and damaged tissue within the brain; (3) enhancing tissue regeneration and angiogenesis, improving the oxygenation of brain tissue; (4) enhancing mitochondrial biogenesis in brain cells, and supporting mitochondrial homeostasis; and (5) helping to enhance the clearance of any pathogens that may infect brain tissue, including viruses such as Herpevirus, and bacteria such as Chlamydia pneumoniae and Spirochetes, which are associated with Alzheimer's Disease and dementia. This is a novel approach to inducing the CAMP gene, which may prove beneficial not only for the treatment of Alzheimer's Disease, but for the treatment of other conditions and diseases as well. In the preferred embodiment, a polytherapy of compounds has been intentionally selected, each of which has already been proven to be safe for humans in extensive testing, since this treatment may be most effective if taken regularly.

The pharmaceutical compositions disclosed herein may utilize one or more active ingredients (and will preferably utilize multiple active ingredients) which may be dissolved, suspended or disposed in various media. Such media may include, for example, various liquid, solid or multistate media such as, for example, emulsions gels or creams. Such media may include liquid media, which may be hydrophobic or may comprise one or more triglycerides or oils. Such media may include, but is not limited to, vegetable oils, fish oils, animal fats, hydrogenated vegetable oils, partially hydrogenated vegetable oils, synthetic triglycerides, modified triglycerides, fractionated triglycerides, and mixtures thereof. Triglycerides used in these pharmaceutical compositions may include those selected from the group consisting of almond oil; babassu oil; borage oil; blackcurrant seed oil; black seed oil; canola oil; castor oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; soy oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; glyceryl tricaprylate/caprate/stearate; saturated polyglycolized glycerides; linoleic glycerides; caprylic/capric glycerides; modified triglycerides; fractionated triglycerides; and mixtures thereof. The use of coconut oil is especially preferred.

Various fatty acids may be utilized in the pharmaceutical compositions disclosed herein. These include, without limitation, both long and short chain fatty acids. Examples of such fatty acids include, but are not limited to, docosahexaenoic acid, caprylic acid, capric acid, lauric acid, butyric acid, and pharmaceutically acceptable salts thereof.

The pharmaceutical compositions disclosed herein may be applied in various manners. Thus, for example, these compositions may be applied as oral, transdermal, transmucosal, intravenous or injected treatments, or via cell-based drug delivery systems. Moreover, these compositions may be applied in a single dose, multi-dose or controlled release fashion.

The pharmaceutical compositions disclosed herein may be manufactured as tablets, liquids, gels, foams, ointments or powders. In some embodiments, these compositions may be applied as microparticles or nanoparticles.

In some embodiments, the pharmaceutically acceptable compositions disclosed herein preferably include a mixture of at least four more preferably at least five, and most preferably at least six materials (preferably active materials) selected from the group consisting of phenylbutyrate, bexarotene, curcumin, resveratrol, retinal, phenylbutyrate, cholecalciferol, fatty acids, and pharmaceutically acceptable salts thereof. In other embodiments, the pharmaceutically acceptable compositions disclosed herein preferably include a mixture of at least four more preferably at least five, and most preferably at least six materials (preferably active materials) selected from the group consisting of phenylbutyrate, bexarotene, curcumin, resveratrol, retinol, phenylbutyrate, cholecalciferol, docosahexaenoic acid, caprylic acid, capric acid, lauric acid, and pharmaceutically acceptable salts thereof.

Various counterions may be utilized in forming pharmaceutically acceptable salts of the materials disclosed herein. One skilled in the art will appreciate that the specific choice of counterion may be dictated by various considerations. However, the use of sodium and hydrochloride salts may be preferred in some applications.

The above description of the present invention is illustrative, and is not intended to be limiting. It will thus be appreciated that various additions, substitutions and modifications may be made to the above described embodiments without departing from the scope of the present invention. Accordingly, the scope of the present invention should be construed in reference to the appended claims.

1. A method for treating an individual, comprising:
administering a pharmaceutically acceptable composition to an individual, wherein the pharmaceutically acceptable composition upregulates cathelicidin antimicrobial peptide (CAMP) gene expression in the individual.

2. The method of claim 1, wherein the CAMP gene expresses a cathelicidin LL-37 peptide.

3. The method of claim 2, further comprising monitoring levels of the cathelicidin LL-37 peptide in the individual.

4. The method of claim 3, further comprising monitoring levels of β-amyloid in the individual.

5. The method of claim 4, further comprising administering a larger dose of the pharmaceutically acceptable composition to the individual, when the L/B<k is detected, where L is a level of cathelicidin LL-37 peptide detected, B is a level of β-amyloid detected, and k is a predetermined threshold value.

6. The method of claim 5, wherein k is 1.

7. The method of claim 1, wherein the individual suffers from Alzheimer's disease.

8. The method of claim 7, wherein the CAMP gene expression occurs in a brain of an individual.

9. The method of claim 8, wherein the CAMP gene expresses a cathelicidin LL-37 peptide and the cathelicidin LL-37 peptide causes a reduction in β-amyloid fibrils in the individual.

10. The method of claim 1, wherein the pharmaceutically acceptable composition comprises a mixture of at least four materials selected from the group consisting of phenylbutyrate, bexarotene, curcumin, resveratrol, retinal, cholecalciferol, fatty acids, and pharmaceutically acceptable salts thereof.

11. The method of claim 1, wherein the pharmaceutically acceptable composition comprises a mixture of at least four materials selected from the group consisting of phenyl butyrate, bexarotene, curcumin, resveratrol, retinal, cholecalciferol, docosahexaenoic acid, caprylic acid, capric acid, lauric acid, and pharmaceutically acceptable salts thereof.

12. The method of claim 1, wherein the pharmaceutically acceptable composition comprises a mixture of at least three materials selected from the group consisting of phenyl butyrate, bexarotene, curcumin, resveratrol, retinal, cholecalciferol, and pharmaceutically acceptable salts thereof.

13. The method of claim 12, wherein the pharmaceutically acceptable composition comprises a mixture of at least five materials selected from the group consisting of phenyl butyrate, bexarotene, curcumin, resveratrol, retinol, cholecalciferol, fatty acids, and pharmaceutically acceptable salts thereof.

14. The method of claim 12, wherein said mixture is dissolved in a hydrophobic liquid medium.

15. The method of claim 1, further comprising diagnosing the individual for Alzheimer's disease.

16. The method of claim 1, further comprising detecting a presence of β-amyloid aggregate accumulation in the individual, wherein β-amyloid aggregate accumulation comprises at least one structure selected from the group consisting of oligomers, fibrils, and plaques.

17. The method of claim 1, further comprising monitoring a level of a cytokine in the microglia tissues of the individual.

18. The method of claim 17, wherein the cytokine is selected from IL-6, IL-8, IL-1α, and TNFα.

19. The method of claim 17, wherein the CAMP gene expresses a cathelicidin LL-37 peptide, wherein the cathelicidin LL-37 peptide complexes with a β-amyloid in the individual.

20. The method of claim 19, wherein the cathelicidin LL-37 peptide-β-amyloid complexation reduces the level of the cytokine.
